(19) United States
(12) Patent Application Publication
(10) Pub. No.: US 2021/0094922 A1
(43) Pub. Date: Apr. 1, 2021

(54) Dyes for Analysis of Soluble Protein Aggregates or Misfolded Protein Oligomers

(71) Applicant: The Penn State Research Foundation, University Park, PA (US)

(72) Inventors: Xin Zhang, State College, PA (US); Yu Liu, State College, PA (US); Charles Wolstenholme, Fairfield, CT (US)

(21) Appl. No.: 16/978,479

(22) PCT Filed: Mar. 7, 2019
(86) PCT No.: PCT/US2019/021175

§ 371 Date: Sep. 4, 2020

Related U.S. Application Data
(60) Provisional application No. 62/639,952, filed on Mar. 7, 2018.

(51) Int. Cl. C07D 233/70; C07D 403/06; C07D 401/14; C07D 409/14; C07D 417/14; C07D 471/04; C07D 407/14; C07F 9/6509; C07F 9/6506; C07D 233/84; G01N 21/64

(52) U.S. Cl. CPC C07D 233/70; C07D 403/06; C07D 401/14; C07D 409/14; C07D 417/14; G01N 21/6428; C07D 407/14; C07F 9/650952; C07F 9/6506; C07D 233/84; G01N 21/6458; C07D 471/04

(57) ABSTRACT

Dye and compositions to monitor the multistep protein aggregation process in both test tubes and live cells are provided. These dyes can detect misfolded protein oligomers and distinguish insoluble protein aggregates from misfolded oligomers. Applications of these dyes include measuring kinetics of protein aggregation, monitoring aggregation of specific proteins in intact live cells, monitoring aggregation of cellular proteome in intact live cells, and tracking the time course of protein aggregation in cells under stress conditions.

### CROSS-REFERENCE TO RELATED APPLICATIONS

This application claims the benefit of U.S. Provisional Patent Application No. 62/639,952, filed on Mar. 7, 2018, which is incorporated by reference in its entirety.

### FIELD OF THE INVENTION

The present invention relates to dyes and compositions for studying protein aggregation processes.

### BACKGROUND OF THE INVENTION

Mechanisms of Protein Aggregation

Environmental stresses and pathogenic mutations of proteins lead to aberrant misfolding and aggregation, causing neurodegenerative diseases, including Alzheimer's disease, Parkinson disease, familial amyloidosis, and amyotrophic lateral sclerosis. Protein aggregation is a multistep process that has been associated with a growing number of human diseases, including neurodegenerative disorders, metabolic disorders, some cancers.¹⁻⁶ Misfolding yields misfolded monomers, which subsequently associate with one another to form misfolded oligomers. Misfolded oligomers evolve into insoluble aggregates in forms of amyloid-β fibrils, amorphous aggregates, or stress granules. Studying the multistep process of protein aggregation, in particular the intermediate misfolded oligomers, is increasingly being recognized as an important field in the biomedical and biochemical communities.

Misfolded Protein Oligomers in Diseases

Protein homeostasis (proteostasis) dynamically adapts to diverse environmental factors and cellular events.¹ To achieve an appropriate level of proteostasis, the endogenous proteome has evolved to maintain a specific balance between the folded, misfolded and aggregate states of its protein components. However, exogenous stress conditions (including environmental perturbations, chemical toxins, and pathogen invasion) impair the integrity of proteostasis by shifting the free energy landscape of protein folding and/or inducing chemical or conformational changes in folded proteins.^{4,7} Failure to maintain proteostasis during stress leads to global misfolding and aggregation of the endogenous proteome, resulting in a series of aberrant conformations that include misfolded proteins in the form of soluble oligomers, disordered or amorphous aggregates, and fibrils containing ordered hydrogen-bonded β-sheet structures. Formation of these structures often leads to the loss of essential functions and the formation of toxic aggregates. Both of these phenomena have been increasingly associated with a growing number of diseases, such as cancer, neurodegeneration, metabolic disorders, cardiovascular disease and inflammation.⁸⁻¹⁰

Decades of studies have delineated the structure, interaction, and activity of proteins in either their natively folded structures or in insoluble aggregates such as amyloid fibrils. However, a variety of intermediate species exist between these two extreme states in the protein folding landscape. Herein, these conformations are collectively termed as misfolded oligomers, including soluble oligomers and pre-amyloidal oligomers whose formation is driven by misfolded proteins. Accumulating evidence suggests that the misfolded oligomers may play key roles in both cell physiology and pathology.¹¹⁻¹⁵ Firstly, they may exert toxicity in diseases. For instance, soluble oligomers, but not the insoluble deposits, can confer synaptic dysfunction in neurodegenerative disorders². Secondly, beneficial functions have been demonstrated for oligomeric prion or prion-like proteins in processes including development, neuroprotection and metabolism³. Finally, they may be implicated in evolution. It has been shown that oligomers formed by prion proteins induce phenotypic changes in evolution⁴. With the existing knowledge, the biomedical community is in need of establishing methods to study misfolded protein oligomers in living cells.

Detection of Protein Aggregation in Live Cells

The need to reliably detect protein aggregation in live cells has promoted the emergence of a group of fluorescent-based methods.

First, chemical dyes (such as the PROTEOSTAT assay kit) can detect intracellular insoluble aggregates. However, this assay requires cell fixation and membrane permeabilization. Therefore, this method is not suited for live cells.

Second, fusion of Fluorescent Proteins (FP) or labeling of fluorescent probes to Protein-of-Interest (POI) has been used to visualize POI'S aggregation by observing fluorescent granules in live cells.¹⁶⁻¹⁹ The limitation is that FP-fused POIs exhibit fluorescence before AND after aggregation (non-fluorogenic), and this non-fluorogenic nature makes these methods not suited to visualize soluble oligomers because these oligomers do not have granular structures, nor visualize protein aggregation in certain subcellular compartments (such as mitochondria and stress granules) because of their granular morphology.

Third, diffusion constants of FP-fused POI can be quantified to differentiate insoluble aggregates from folded proteins.^{20,21} However, such assays may not easily distinguish misfolded oligomers from folded proteins because both exhibit similar diffusion constants.

Finally, fluorescence resonance energy transfer (FRET) of FP-fused POI has been used to distinguish misfolded oligomers from folded proteins.²¹⁻²³ However, this method is laborious and can encounter complications because these two conformations may exhibit similar FRET signals. Thus, despite efforts in past few decades, no simple and direct method is available to directly visualize the multistep process of how a POI aggregates in live cells.

Fluorescent Protein Chromophores

Fluorescent proteins (FPs) have been widely used as genetic tags to provide spatial and temporal information of a protein-of-interest (POI) in live organisms.^{25,26} Since its discovery, GFP has been used for various biological applications.^{38,39} Variations of the GFP chromophore, 4-hydroxybenzylidene-imidazolinone (HBI), have expanded FPs with diverse photophysical properties, including spectral range, quantum yield, photostability, and photoswitchability.²⁴ These chromophores, however, become mostly non-fluorescent when synthesized outside their protein cavity, largely due to rapid non-radiative decay via twisted-intramolecular charge transfer (TICT).^{28,29}

Although such behavior undermines the potential of FP analogues as a valuable class of fluorophores with broad applications, both chemical and biological restriction of TICT restores fluorescence of synthetic FP chromophores. Inspired by this property, fluorophores have been derived from HBI to be fluorescent with supreme photophysical properties. In addition, FP analogues locked in supra-molecular hosts,²⁹ metal-organic framework,³⁰ aggregated solid,³¹ and host proteins³² have been reported to fluoresce strongly. Applications of HBI analogues in biological imaging are represented by visualization of RNA aptamers and DNA quadruplex.³³⁻³⁷ Beyond these applications, these FP mimics are rarely reported in the detection of other biological processes in live cells.

### SUMMARY OF THE INVENTION

The present invention designs, synthesizes and applies analogues of FP chromophores as fluorescent probes to visualize the multistep process of protein aggregation in live cells.

FP chromophores are used to visualize protein misfolding and aggregation, using turn-on fluorescence, both in test tube and in live cells. Thus, the inventors sensitize FP chromophores, whose TICT can be inhibited in the rigid environment within protein aggregates to turn on fluorescence (FIG. 1).

One aspect of the present invention is directed to a compound of Formula I:

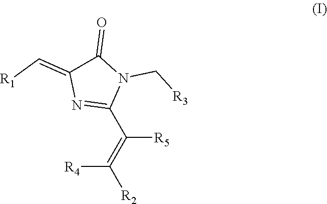

wherein:

R₁, R₂, and R₅ are independently selected from the moieties of Group 1, and R₄ is —H;
R₁, R₂, and R₄ are independently selected from the moieties of Group 1, and R₅ is —H;
R₁ and R₂ are independently selected from the moieties of Group 1, R₄ is —H, and R₅ is —H;
R₁ and R₂ are independently selected from the moieties of Group 1, R₄ is —H and R₅ is —CH₃;
R₁ and R₂ are independently selected from the moieties of Group 1, R₁ is —CH₃ and R₅ is —H; or
R₁, R₂, R₄, and R₅ are independently selected from the moieties of Group 1; and wherein the moieties of Group 1 are selected from the group consisting of

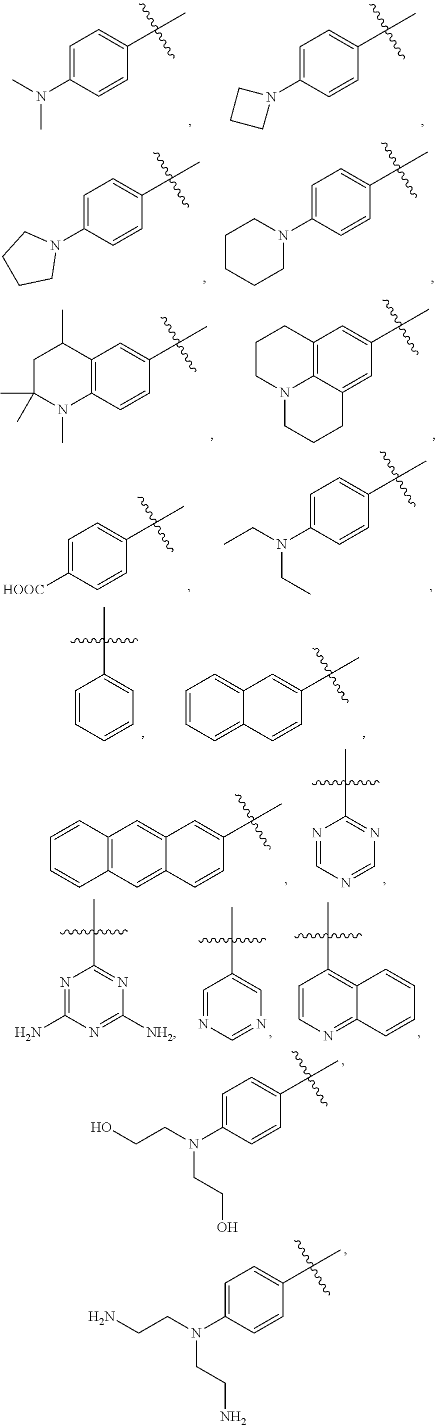

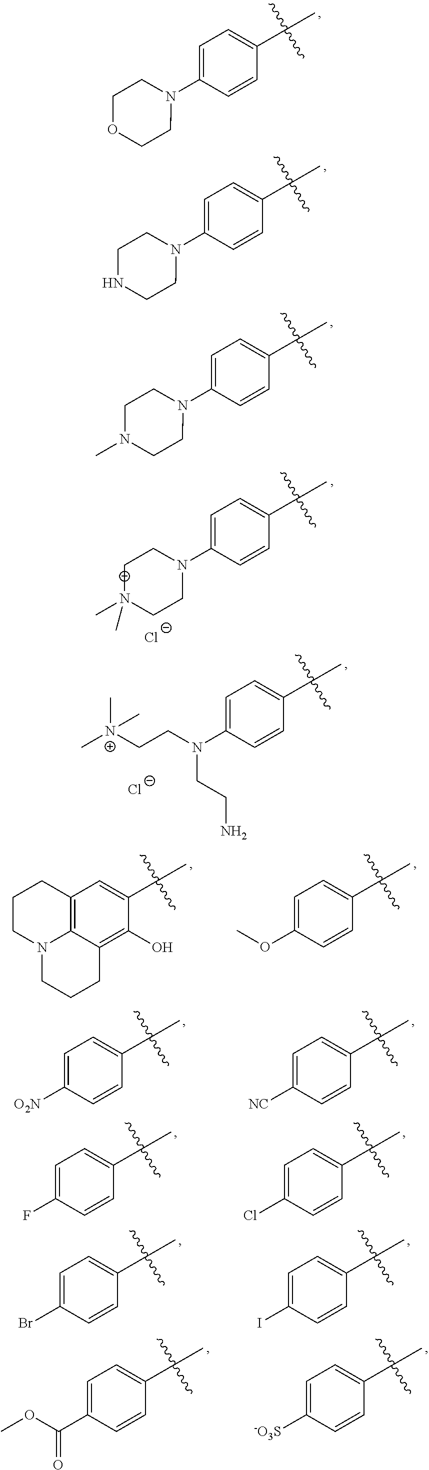

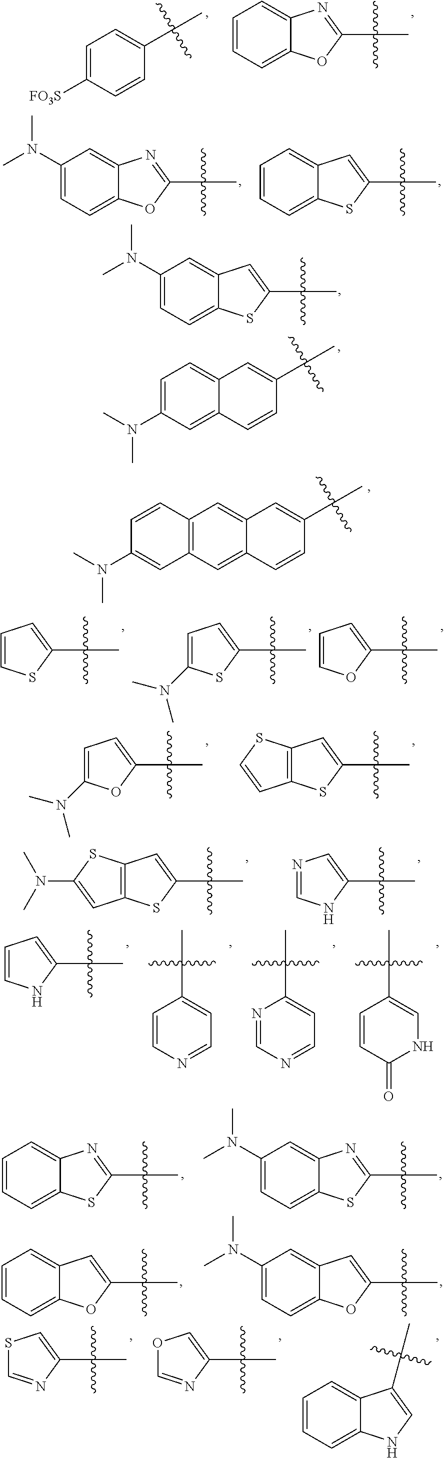

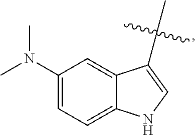 [SMILES: *c1c[nH]c2ccc(N(C)C)cc12]

wherein substituents may be at para-, meta-, or ortho- positions on the aromatic ring, even if shown in only a single position above;
wherein R₃ is a directing moiety that binds and bioconjugates at least one biological target, wherein R₃ is selected from the group consisting of:

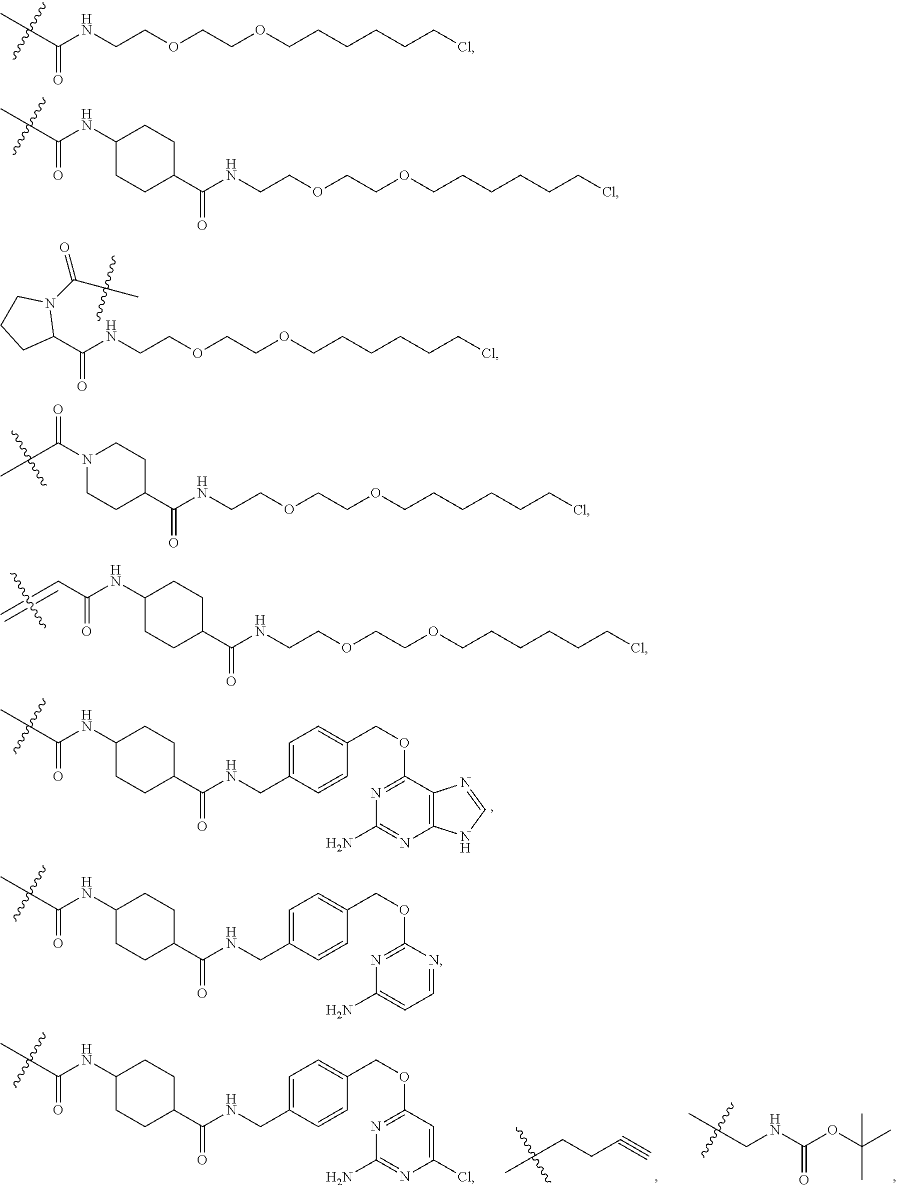

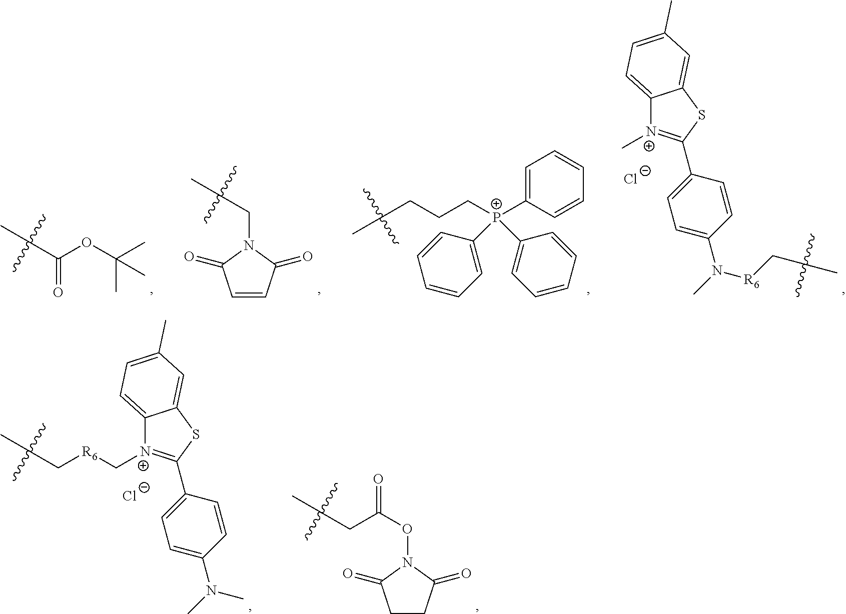

wherein R₆ is selected from the group consisting of

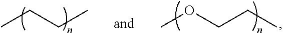

and wherein n is 0, 1, 2, 3, 4, or 5.

Another aspect of the present invention is directed to a compound of Formula II:

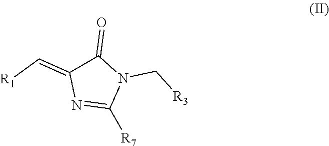

wherein
R₁ and R₇ are independently selected from a moiety of Group 1; or
R₁ is a moiety of Group 1, R₇ is a moiety of Group 2, and R₈ and R₉ are independently selected from —H, —CH₃, and a moiety of Group 1; wherein
a moiety of Group 1 is selected from the group consisting of:

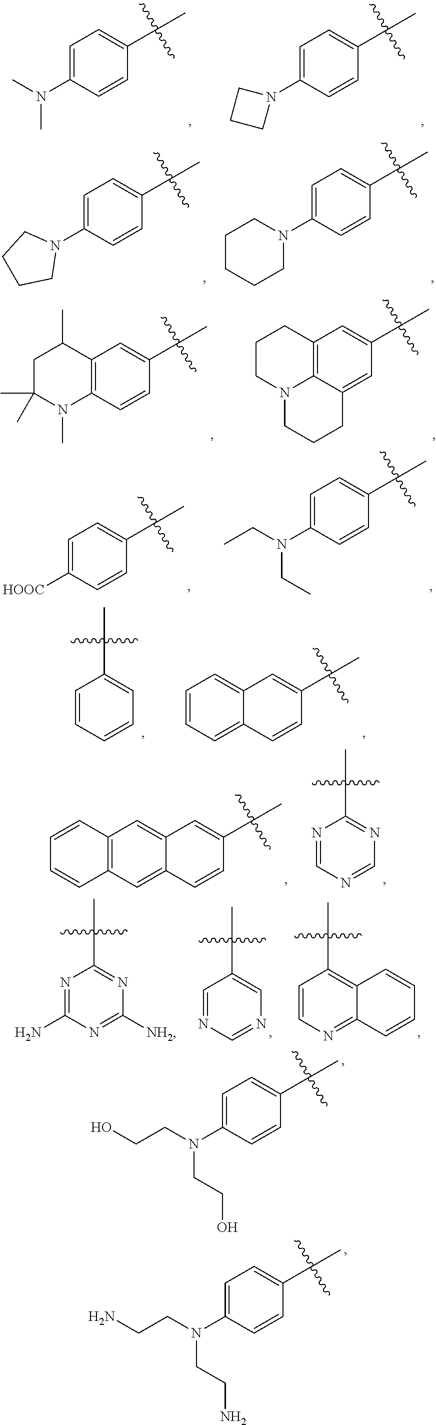

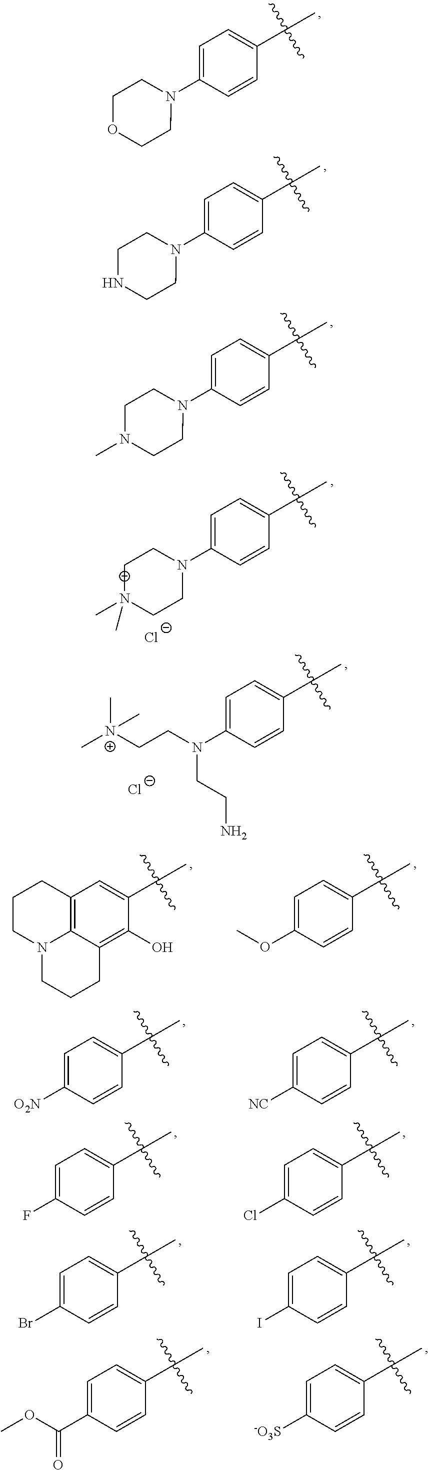

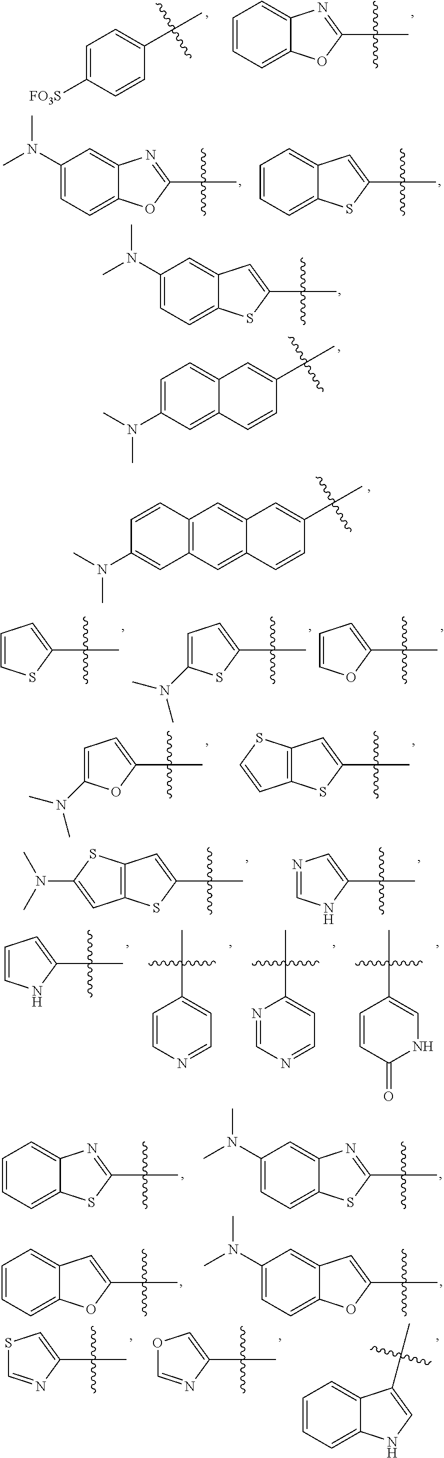

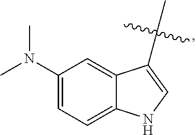 [SMILES: *c1c[nH]c2ccc(N(C)C)cc12]

and wherein
a moiety of Group 2 is selected from the group consisting of:

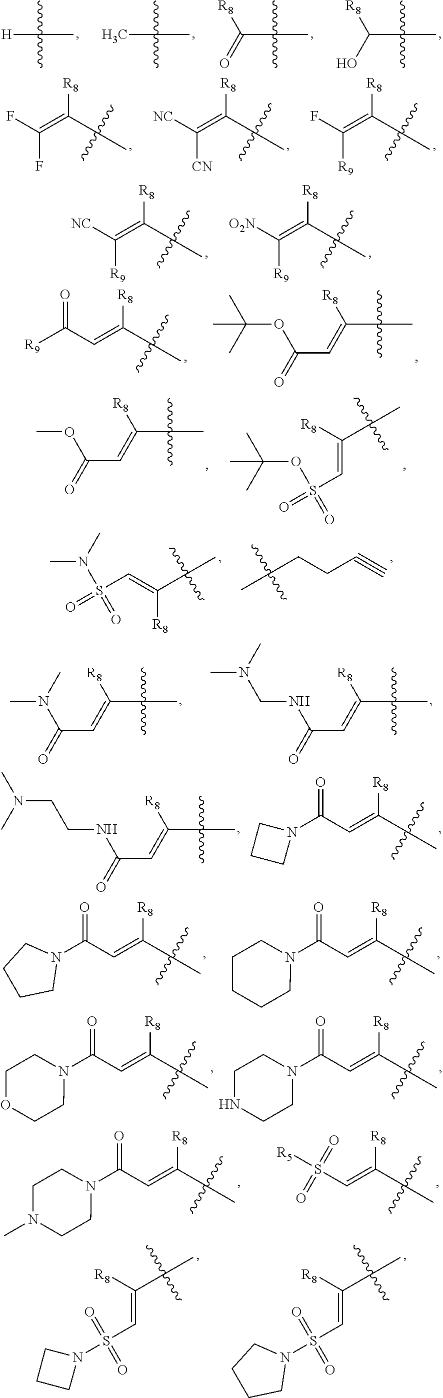

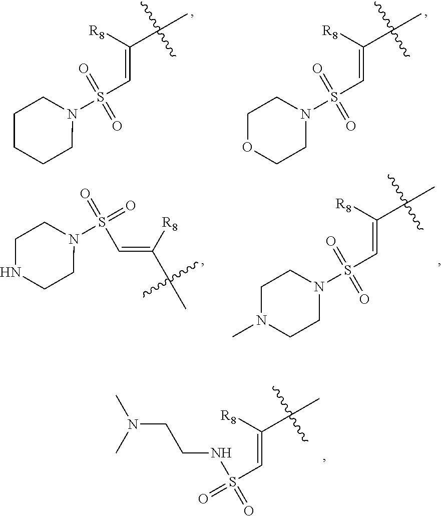

wherein substituents may be at para-, meta-, or ortho- positions on the aromatic ring of Group 1 and Group 2, even if shown in only a single position above; and wherein R3 is a directing moiety to bind and bioconjugate to biological targets, selected from the group consisting of:

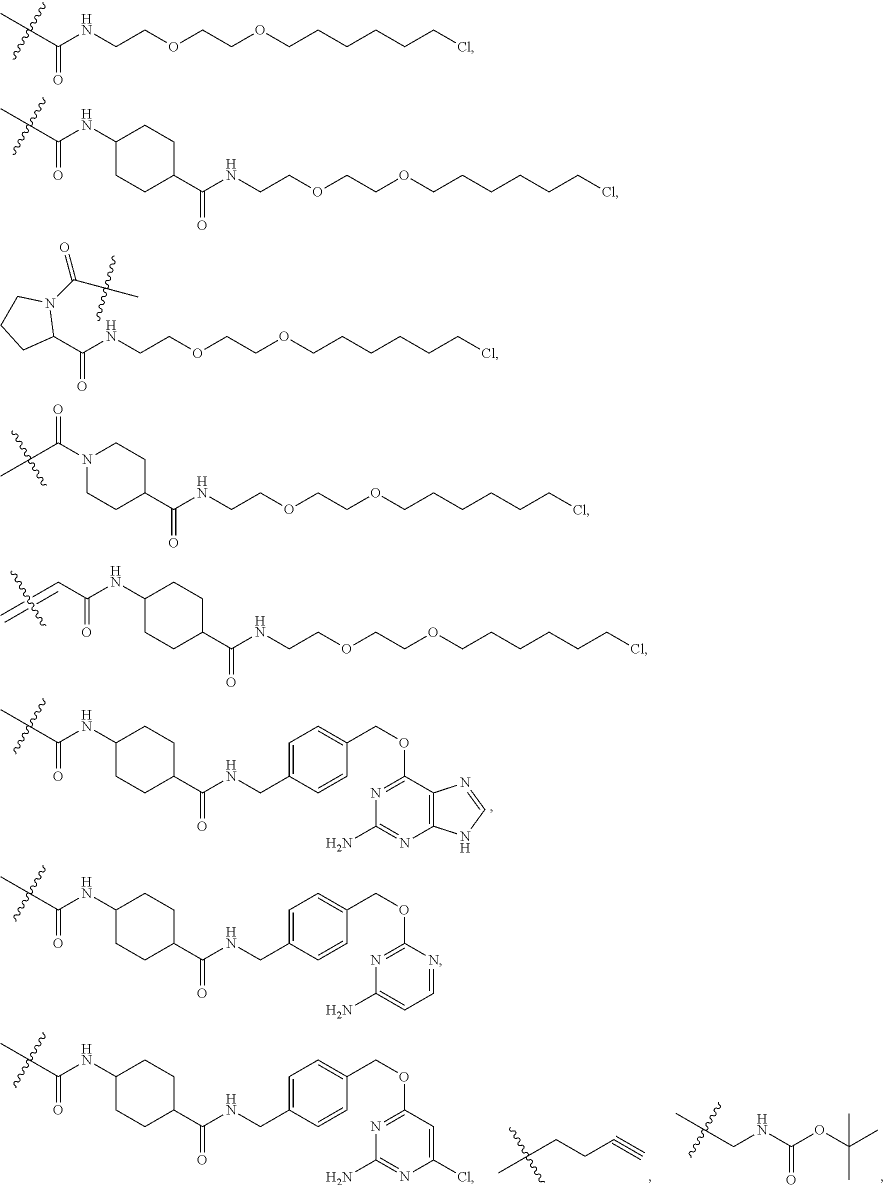

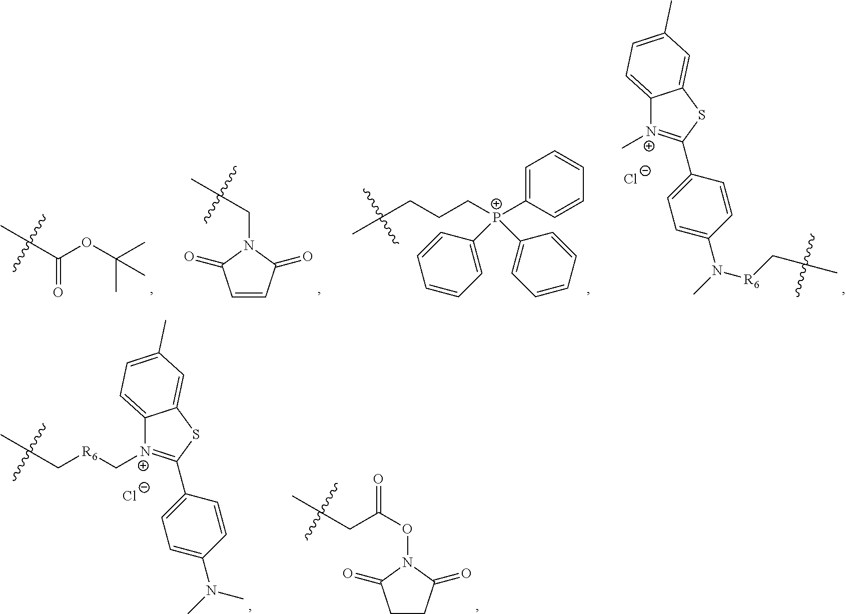

wherein R₆ is selected from the group consisting of

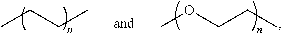

and wherein n is 0, 1, 2, 3, 4, or 5.

Another aspect of the present invention is directed to one of the compounds in Table 1.

Another aspect of the present invention is directed to a method for detecting at least one of aggregated protein, misfolded protein, and amyloid fiber in a protein of interest. The method includes performing a first measurement of fluorescence intensity of a protein of interest; adding to the protein of interest a fluorescent protein chromophore; and performing a second measurement of fluorescence intensity of the protein of interest. The increased fluorescence is indicative of at least one of aggregation, misfolding, and amyloid fiber in the protein of interest.

In one embodiment, the fluorescent protein chromophore is a compound selected from the previously listed compounds.

In another embodiment, the method is conducted in a cell in vivo.

In another embodiment, the method is conducted in vitro.

In another embodiment, the method further includes purifying the protein of interest prior to the first measurement.

In another embodiment, the fluorescent protein chromophore includes a thioflavin-T guiding group or a tert-butyloxycarbonyl guiding group.

Another aspect of the present invention is directed to a method for detecting at least one of aggregated protein, misfolded protein, and amyloid fiber in a protein of interest. The method includes performing a first measurement of fluorescence intensity of a standard protein; adding to a protein of interest a fluorescent protein chromophore; performing a measurement of fluorescence intensity of the protein of interest; and comparing the fluorescence intensity of the standard protein with the fluorescence intensity of the protein of interest. The increased fluorescence of the protein of interest is indicative of at least one of aggregation, misfolding, and amyloid fiber in the protein of interest.

Another aspect of the present invention is directed to a kit for detecting at least one of aggregated protein, misfolded protein, and amyloid fiber. The kit includes one or more fluorescent protein chromophores previously listed of a known concentration in a stock solution, one or more standard protein samples that form aggregated protein, misfolded protein, and amyloid fiber and optionally, instructions for use in detecting at least one of one of the aggregated protein, misfolded protein, and amyloid fiber.

In one embodiment, the stock solution is dimethyl sulfoxide or ethanol.

Another aspect of the present invention is directed to the use of the previously listed compounds to detect insoluble aggregates.

In one embodiment, the moiety of Group 2 can be an alkyl group wherein the alkyl group is saturated or unsaturated, linear or branched, substituted or unsubstituted.

Another aspect of the present invention is directed to a method for assaying aggregation of proteins in live cells is provided (AggGlow method). The kit includes in packaged combinations: (a) one or more of the previously listed compounds, and (b) instructions for using the compound for assaying aggregation of proteins in live cells.

Another aspect of the present invention is directed to a multi-dye composition comprising at least two dyes that are excited at different wavelengths is provided (AggGlow method).

Another aspect of the present invention is directed to a kit for assaying aggregation of a Halo-Tag fusion protein in live cells is provided (AggTag method). The kit includes in packaged combinations: (a) one or more of the previously listed compounds, and (b) instructions for using the compound for assaying aggregation of proteins in live cells.

Another aspect of the present invention is directed to a kit for assaying aggregation of a SNAP-Tag fusion protein in live cells is provided (AggTag method). The kit comprises in packaged combinations: (a) one or more of the previously listed compounds, and (b) instructions for using the compound for assaying aggregation of proteins in live cells.

Another aspect of the present invention is directed to a multi-dye composition including at least two dyes that are (a) excited at different wavelengths and (b) conjugated to SNAP-Tag or Halo-Tag fusion proteins (AggTag method). The dyes are selected from the previously listed compounds.

Other aspects and advantages of the invention will be apparent from the following description, drawings and the appended claims.

### BRIEF DESCRIPTION OF THE DRAWINGS

The above and other objects, aspects, features, advantages and possible applications of the present innovation will be more apparent from the following more particular description thereof, presented in conjunction with the following drawings. Like reference numbers used in the drawings may identify like components.

FIG. 1 shows an example of fluorescent protein chromophores modulated to serve as fluorogenic probes to detect protein aggregates.

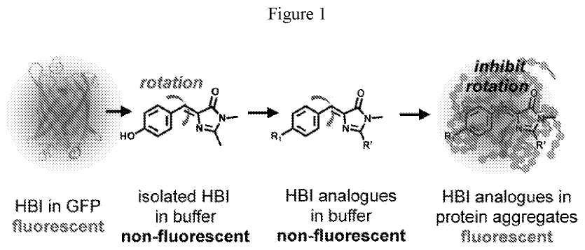

FIG. 2 shows an analogue of the Kaede chromophore is fluorescent in viscous solvent and crystal. (a) Chemical structures of the FP chromophore mimics in this work. (b) Relative brightness of fluorophores in glycerol. Calculation of brightness is shown in Table 2. (c) Crystal of 3 exhibits red fluorescence. (d) The planar structure of the fluorophore moiety of 3 in the crystal (the t-butyl group is omitted for simplicity).

FIG. 3 shows In vitro detection of protein aggregates. (a) Protein aggregates provide a crowded environment to restrict rotational motion of 3 and turn on its fluorescence. (b) Aggregation kinetics of α-synuclein as measured by 3 and ThT. (c) Heat-induced aggregation of SOD1(V31A) increases fluorescence intensity of 3. (d) Kinetics of fluorescence increase is faster than that of turbidity increase during aggregation of SOD1(V31A) at 54.5° C.

FIG. 4 shows The FP chromophore analogue 4 enables a fluorogenic method (AggTag) to detect aggregates of specific proteins in live cells. (a) Diagram of the fluorogenic AggTag method that detects POI aggregation in live cells. (b) Structure of 4. (c) Aggregation of the Htt 97Q-Halo-4 conjugate induces fluorescence increase. (d) Fluorescent image of cell lysate from cells expressing Q0-Halo or Q97-Halo labeled by 4 after fractionation. Quantification of fluorescent intensity is shown in FIG. 18c. T: total lysate; S: supernatant; I: insoluble fraction.

FIG. 5 shows The AggTag method detects stress-induced protein aggregates that are invisible using non-fluorogenic methods. (a) Fluorogenic detection of NaAsO₂-induced SOD1(V31A) aggregation in HEK293T cells. (b) NaAsO2 treatment leads to more SOD1(V31A) aggregation as confirmed by fractionation.

FIG. 6 shows fluorescence response of FP chromophore analogues in H₂O with increasing concentrations of glycerol. FP analogues (20 μM) were prepared in glycerol:H₂O mixture with increasing glycerol concentrations. In addition, fluorescence intensity in 1,4-dioxane was measured to evaluate their response to polarity. Fluorescence measurement was carried out using a Tecan infinite M1000Pro fluorescence microplate reader at excitation and emission wavelengths as indicated. All readings were normalized against the fluorescence intensity in 100% glycerol as 1. Error bars: standard error (n=3).

FIG. 7 shows normalized excitation spectra of FP chromophore analogues in glycerol. FP analogues (20 μM) were prepared in glycerol. Spectra were collected with emission wavelength of 420 nm for 1, 525 nm for 2, 620 nm for 3, 3a and 3b. All measurements were carried out using a Tecan infinite M1000Pro fluorescence microplate reader.

FIG. 8 shows normalized emission spectra of FP chromophore analogues in glycerol. FP analogues (20 μM) were prepared in glycerol. Spectra were collected with excitation wavelength of 370 nm for 1, 455 nm for 2, 530 nm for 3 and 3b, 485 nm for 3a. All measurements were carried out using a Tecan infinite M1000Pro fluorescence microplate reader.

FIG. 9 shows absorbance spectra of FP chromophore analogues in glycerol. FP analogues (10 μM) were prepared in glycerol. Spectra were collected with 10 mm quartz cuvette. All measurements were carried out using Agilent 300 UV-Vis spectrophotometer.

FIG. 10 shows the crystal structure of 3. (a) The crystal of 3 is fluorescent. The crystals were looped out and transferred to a 35 mm glass bottom dish. Images were taken using an inverted Biorad ZOE fluorescent cell imager. This data suggests that 3 in the crystal structure should adopt conformation that is fluorescent. Reason of the dark image in the bright field is because light of an inverted microscope could not pass through the thick crystal. (b) Crystal structure and packing diagram of 3, with thermal ellipsoids drawn at 50% probability level.

FIG. 11 shows computational analyses of 3 validate its planar S1 excited state structure as the fluorescent state and identify the charge separation at S1 excited state. (a) The optimized geometries of 3 in ground state and excited state. (b-c) A dihedral angle analysis confirmed planar structures of 3 in both ground state (GS) and excited state (ES). (d) Charge density difference isosurfaces (isovalue=0.0004) at the minimum energy conical intersection between ground state and S1 excited state. Positive isosurfaces are blue and indicate electron withdraw. Negative isosurfaces are cyan and indicate electron donation. This data suggests that group A is the primary electron donor that contributes to charge separation at S1 excited state.

FIG. 12 shows fluorogenic detection of mature α-synuclein fibers using 3 or ThT. Aggregation of α-synuclein was carried out using method described in the Supporting Information. Subsequently, half of reaction mixture was loaded on a 96-well plate to measure fluorescence intensity of 3 (a) or ThT (b) for the reaction mixture (T). The other half was centrifuged at 21,000 g for 30 minutes at 4° C. The supernatant fraction (S) and pellet fraction (P, after resuspension with a volume that was equal to that of the S fraction) were loaded on a 96-well plate to measure fluorescence intensity. 10 μM 3 or ThT was also incubated in buffer as a control. Parameters for fluorescence measurement: 3 (Eₓ=530 nm/Eₘ=600 nm) and ThT (Eₓ=440 nm/Eₘ=480 nm). Error bars: standard error (n=3).

FIG. 13 shows formation of soluble oligomers and mature α-synuclein fibers. (a) Photo-induced cross-linking experiment observed soluble oligomers of α-synuclein at an 8-h time point. (b) Electron micrographs of α-synuclein fibrils formed at 72 h. Images were taken at different resolutions. Scale bars: 200 nrn, 500 nm, 1 μm, and 5 μm.

FIG. 14 shows fluorogenic detection of SOD1(V31A) aggregates using 3. (a) SOD1(V31A) aggregation (42 μM) was induced at 59° C. for 30 min in buffer (50 mM Tris-HCl, pH 7.5, 100 mM NaCl, 83 mM EDTA), in the presence of 3 (21 μM). Reaction mixture was illuminated by UV trans-illuminator before and after centrifugation at 21,000 g for 10 min at 4° C. (b) Fluorescence intensity of SOD1(V31A) aggregates (42 μM) in the presence of varying concentrations of 3. 100 μM of 3 in buffer was used as a control. Parameters for fluorescence measurement: 3 (Eₓ=530 nm/Eₘ=600 nm). Error bars: standard error (n=3).

FIG. 15 shows spectroscopic characterization of 4. (a) Fluorescence response of 4 in H₂O with increasing concentrations of glycerol. 20 μM of 4 was prepared in glycerol:H₂O mixture with increasing glycerol concentrations. In addition, its fluorescence intensity in 1,4-dioxane was measured. All readings were normalized against the fluorescence intensity in 100% glycerol as 1. Error bars: standard error (n=3). (b-c) Normalized excitation and emission spectra of 4 in glycerol. 20 M of 4 was prepared in glycerol. (d) Absorbance spectra of FP chromophore analogues in glycerol. FP analogues (10 μM) were prepared in glycerol. Spectra were collected with 10 mm quartz cuvette. Parameters for fluorescence measurement: 4 (Eₓ=530 nm/Eₘ=630 nm).

FIG. 16 shows fluorescence response of SBD, CCVJ, and 4 to BSA and SDS. 20 M of fluorophores were incubated in H₂O, glycerol, dioxane, BSA (2 mg/mL), or SDS (0.2%). Intensities were normalized against the value in either 100% glycerol as 1 for CCVJ and 4, or dioxane as 1 for SBD. Parameters for fluorescence measurement: SBD (Eₓ=450 nm/Eₘ=545 nm), CCVJ (Eₓ=450 nm/Eₘ=500 nm), 4 (Eₓ=530 nm/Eₘ=630 nm). Error bars: standard error (n=3).

FIG. 17 shows confocal fluorescent images of Htt Q0-Halo, Hit Q97-Halo and Htt Q97-mCherry in HEK293T cells. (a) Schematic diagram for classic non-fluorogenic imaging approaches to visualize protein aggregates in live cells, via identification of fluorescent punctate structures due to the slower diffusion rate of insoluble aggregates than soluble proteins. (b) The Htt Q0-Halo, Htt Q97-Halo and Htt Q97-mCherry proteins were transiently transfected and expressed HEK293T cells for 24 h. 1 μM TMR Halo-Tag ligand was added during protein expression to covalently label the Htt Q0-Halo and Htt Q97-Halo proteins. In cells expressing Htt Q97 proteins, obvious punctate structures (white arrows) were found in the background of diffuse fluorescence. Whereas, only diffuse fluorescence was observed in cells expressing the Htt Q0 protein (upper panel). Red: HaloTag-TMR or mCherry. Blue: nucleus stained by Hoechst 33342. Scale bar: 10 μm.

FIG. 18 shows fluorogenic detection of Htt Q97-Halo aggregation in HEK293T cells. (a) The dual-probe live cell imaging experiment. Halo-tag fusion proteins were labeled in the presence of both the non-fluorogenic coumarin ligand (1 μM) and 4 (1 jM). The newly synthesized Htt-Halo proteins were equally labeled with either the coumarin ligand or 4. The coumarin ligand is fluorescent both before and after aggregation of Htt-Halo. By contrast, signal of 4 exclusively arises from Htt-Halo aggregates. (b) Dual probes labeling confirms expression of proteins. The Htt Q0-Halo and Htt Q97-Halo proteins were transiently transfected and expressed in HEK293T cells for 24 h, in the presence of 1 μM coumarin Halo-Tag ligand and 1 μM of 4. In cells expressing Htt Q97-Halo proteins, punctate structures in the coumarin channel coincided with the turn-on fluorescence of 4. Whereas, only diffuse coumarin fluorescence was observed in cells expressing the Htt Q0-Halo protein and no turn-on fluorescence of 4 was found. (c) The Htt Q97 protein, without a Halo-Tag, was transiently transfected and expressed in HEK293T cells for 24 h, in the presence of 1 μM of 4. No turn-on fluorescence of 4 was found. Red: fluorescence from the SOD1(V31A)-Halo-4 conjugate. Blue: coumarin Halo-Tag ligand in (a) or nucleus stained by Hoechst 33342 in (b). Scale bar: 10 μm. (d) Fluorescent intensity of cell lysates from samples as represented in FIG. 4d. Error bars: standard error (n=3).

FIG. 19 shows confocal images of Halo-Tag in HEK293T cells treated with DPBS or NaAsO₂ (50 M, 24 h). During protein expression, medium is supplemented with 1 μM of coumarin ligand and 1 μM of 4 to enable a simultaneous dual-probe labeling to Halo-Tag. Note: Halo-Tag locates in both cytoplasm and nucleus. Blue: fluorescence from the Halo-Tag⋅coumarin conjugate. Red: fluorescence from the Halo-Tag-4 conjugate. Scale bar: 10 μm.

FIG. 20 shows SOD1(V31A)-Halo fusion protein formed insoluble aggregates in cells treated with NaAsO₂ (extended from FIG. 5b). Protein concentration of cell lysates was determined by a Bradford assay using pre-quantified BSA as a standard. Lysates of cells with or without NaASO₂ stress were normalized to the same concentration and centrifuged at 21,000 g for 30 min at 4° C. Insoluble fraction of cell lysate was resuspended in SDS-PAGE loading buffer and resolved in SDS-PAGE gel. The SOD1(V31A) was either visualized via fluorescence from 4 (FL on the right) or coomassie blue stain (CB on the right).

FIG. 21 shows purified SOD1(V31A)-Halo fusion protein formed insoluble aggregates and turned on fluorescence of 4 under heat with time and temperature dependence. (a) Purified SOD1(V31A)-Halo and Halo-Tag proteins (42 μM), both of which were conjugated with 4 (21 μM), were incubated at varying temperatures (25, 37, 38.9, 41.8, 45.6, 50.7, 54.5, 57.2, and 59° C.). As control samples, 4 (21 μM) was prepared in buffer, 1,4-dioxane, glycerol, BSA (2 mg/mL). Fluorescence intensity was recorded at Eₓ=530 nm/Eₘ=600 nm. All readings were normalized to intensity of the Halo-Tag-4 conjugate at 25° C. Fluorescence of protein samples was also visualized via UV transilluminator. In addition, protein samples were centrifuged at 21,000 g for 30 min at 4° C. to analyze their insoluble fraction using fluorescence of 4 as signal on SDS-PAGE gel. While obvious insoluble aggregates were only found at 57.2 and 59° C., turn-on fluorescence of the SOD1(V31A)-Halo-4 conjugate was observed at lower temperatures. This data suggests that 4 is able to fluoresce prior to formation of insoluble aggregates. This fluorescence was not originated from aggregation of Halo-Tag domain, as evidenced by the low fluorescence of the Halo-4 conjugate and lack of significant insoluble fraction at these temperatures. Error bars: standard error (n=3). (b) Purified SOD1(V31A)-Halo and Halo-Tag proteins (42 M) were conjugated with 4 (21 μM) and incubated at 54.5° C. Fluorescence was recorded using Agilent Carey Eclipse fluorescence spectrophotometer with temperature control. Buffer condition: 50 mM Tris-HCl, pH 7.5, 100 mM NaCl, 83 mM EDTA. Parameters for fluorescence measurement: 4 (Eₐ=530 nm/Eₘ=600 nm).

FIG. 22 shows fluorescence of the SOD1(V31A)-Halo-4 conjugate originates from protein misfolding. (a) Purified SOD1(V31A)-Halo (42 μM) was conjugated with 4 (21 μM) and incubated at 54.5° C. Fluorescence was recorded using Agilent Carey Eclipse fluorescence spectrophotometer with temperature control. Turbidity was recorded using Agilent 300 UV-Vis spectrophotometer. Buffer condition: 50 mM Tris-HCl, pH 7.5, 100 mM NaCl, 83 mM EDTA. (b) Kinetics of fluorescence increases from the SOD1(V31A)-Halo-4 conjugate (red curve) was compared to the kinetics of protein misfolding (black curve) as measured by Tryptophan fluorescence change. Fluorescence was recorded using Agilent Carey Eclipse fluorescence spectrophotometer with temperature control. Experiments were carried out at 54.5° C. Parameters for fluorescence measurement: 4 (Eₓ=530 nm/Eₘ=600 nm) and Trp (Eₓ=290 nm/Eₘ=330 and 345 nm). Error bars: standard error (n=3).

FIG. 23 shows fluorescence of 3 in HEK-293T cells treated with 5 μM MG132 for 24 h, 37° C., 5% CO₂ (middle panel) and in HeLa cells treated with 1 M 17-AAG for 24 h, 37° C., 5% CO₂ (right panel). Untreated cells remain dark as shown in right panel.

FIG. 24 shows that 5 reports on insoluble aggregates formed by SOD-A4V-Halo (A). (B-C) Excitation and emission spectra of 4 (with misfolded oligomers of SOD1-A4V-Halo) and 5 (with insoluble aggregates of SOD1-A4V-Halo). (D-E) When treated with 5 M MG132 for 8 h, 37° C., 5% CO₂, HEK-293T cells expressing SOD-A4V-Halo protein shows fluorescence of 4 (D) but not 5 (D). (F-G) HEK-293T cells expressing Htt-110Q-Halo protein shows fluorescence of 4 (D) and 5 (D). (H) HEK-293T cells expressing Halo and 110Q-mCherry did not show fluorescence of 5.

FIG. 25 shows probes for SNAP-tag conjugation. (A) Chemical scaffold of SNAP-based probes. (B) Linkers with varying length or rigidity (6-11). (C) Synthesis of SNAP-conjugating probes. (D) Quantum yield of P1 probes when conjugated with folded SNAP-tag.

### DETAILED DESCRIPTION OF THE INVENTION

### Definitions

While the terms used herein are believed to be well understood by one of ordinary skill in the art, definitions are set forth herein to facilitate explanation of the subject matter disclosed herein.

Unless defined otherwise, all technical and scientific terms used herein have the same meaning as commonly understood by one of ordinary skill in the art to which the subject matter disclosed herein belongs. Although any methods, devices, and materials similar or equivalent to those described herein can be used in the practice or testing of the presently disclosed subject matter, representative methods, devices, and materials are described herein.

The terms “a,” “an,” and “the” refer to “one or more” when used in this application, including the claims. The use of the word “a” or “an” when used in conjunction with the tenn “comprising” in the claims and/or the specification may mean “one,” but it is also consistent with the meaning of “one or more,” “at least one,” and “one or more than one.”

All references to singular characteristics or limitations of the present disclosure shall include the corresponding plural characteristic(s) or limitation(s) and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The methods and devices of the present disclosure, including components thereof, can comprise, consist of, or consist essentially of the essential elements and limitations of the embodiments described herein, as well as any additional or optional components or limitations described herein or otherwise useful.

Unless otherwise indicated, all numbers expressing physical dimensions, quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently disclosed subject matter.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The following description is of exemplary embodiments that are presently contemplated for carrying out the present invention. This description is not to be taken in a limiting sense, but is made merely for the purpose of describing the general principles and features of the present invention. The scope of the present invention is not limited by this description.

As noted above, embodiments apply an FP chromophore to visualize protein misfolding and aggregation, using turn-on fluorescence, both in test tube and in live cells.

The present invention provides dyes, reagents and methods useful for detection of misfolded protein oligomers and insoluble protein aggregates in vitro and in vivo. In some embodiments, the invention provides a family of probes containing an imidazolinone core structure.

The probes of the invention are useful for generating fluorescence signals that depend upon the presence of an aggregated form of a protein, while conveying minimal levels of signals when only the native form of the protein is present.

Many of these probes exhibit large variation in their excitation and emission wavelength thereby allowing for potential multicolor detection of protein aggregation and sequential imaging.

Specific embodiments of FP chromophores are reported herein. Some of these FP chromophores are included in Table 1, below. In each cell of the table, a compound and its assigned compound number are presented:

TABLE 1

| Compound | |
|---|---|
| Number | Compound |
| 1 | 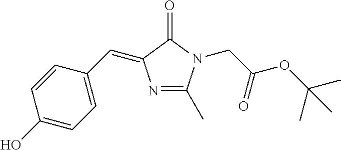 [SMILES: CC1=N/C(=C\c2ccc(O)cc2)C(=O)N1CC(=O)OC(C)(C)C] |
| 2 | 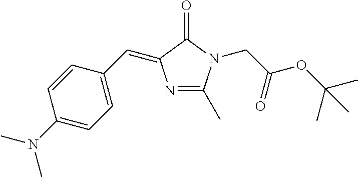 [SMILES: CC1=N/C(=C\c2ccc(N(C)C)cc2)C(=O)N1CC(=O)OC(C)(C)C] |
| 3 | 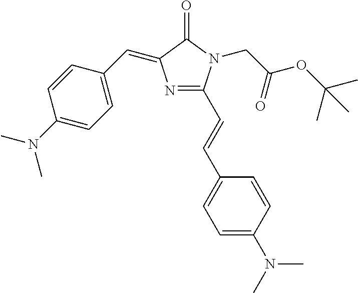 [SMILES: CN(C)c1ccc(/C=C2N=C(/C=C/c3ccc(N(C)C)cc3)N(CC(=O)OC(C)(C)C)C\2=O)cc1] |
| 3a | 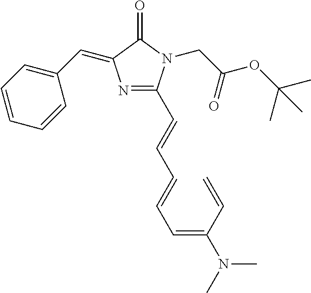 [SMILES: C=C\C(=C/C=C/C=C/C1=NC(=C\c2ccccc2)/C(=O)N1CC(=O)OC(C)(C)C)N(C)C] |
| 3b | 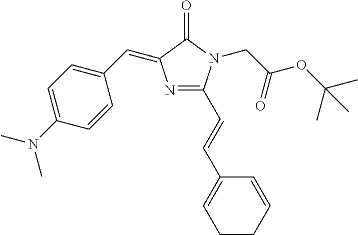 [SMILES: CN(C)c1ccc(/C=C2N=C(/C=C/C3=CCCC=C3)N(CC(=O)OC(C)(C)C)C\2=O)cc1] |
| 4 | 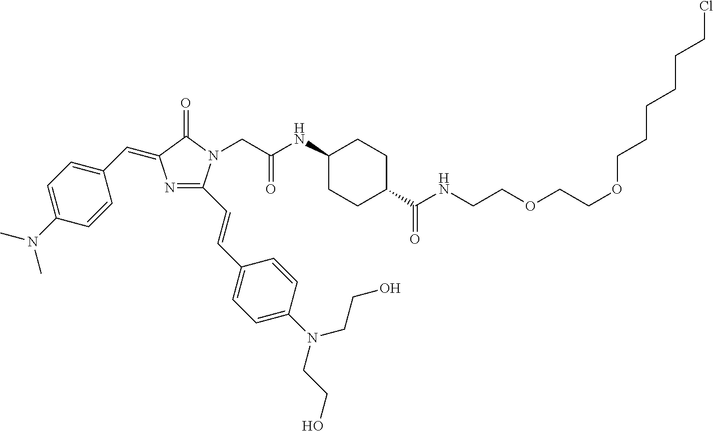 [SMILES: CN(C)c1ccc(/C=C2N=C(/C=C/c3ccc(N(CCO)CCO)cc3)N(CC(=O)N[C@H]3CC[C@H](C(=O)NCCOCCOCCCCCCCl)CC3)C\2=O)cc1] |
| 5 | 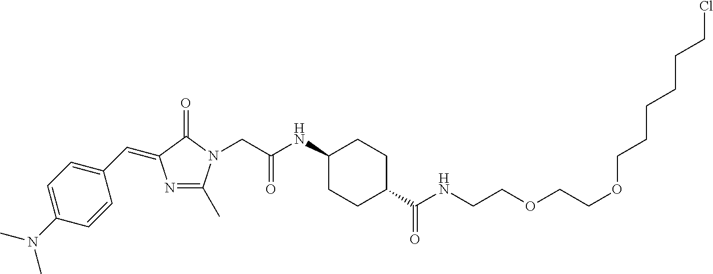 [SMILES: CC1=N/C(=C\c2ccc(N(C)C)cc2)C(=O)N1CC(=O)N[C@H]1CC[C@H](C(=O)NCCOCCOCCCCCCCl)CC1] |
| 6 | 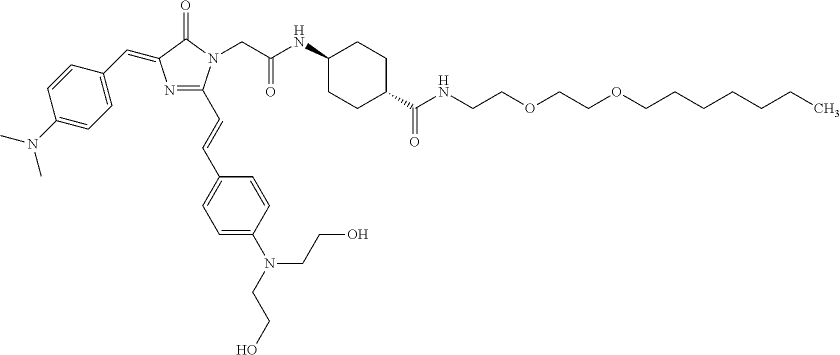 [SMILES: CCCCCCCOCCOCCNC(=O)[C@H]1CC[C@H](NC(=O)CN2C(=O)/C(=C/c3ccc(N(C)C)cc3)N=C2/C=C/c2ccc(N(CCO)CCO)cc2)CC1] |
| 7 | 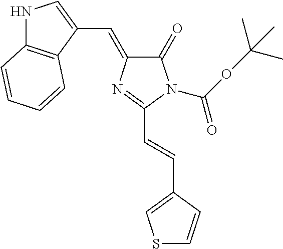 [SMILES: CC(C)(C)OC(=O)N1C(=O)/C(=C/c2c[nH]c3ccccc23)N=C1/C=C/c1ccsc1] |
| 8 | 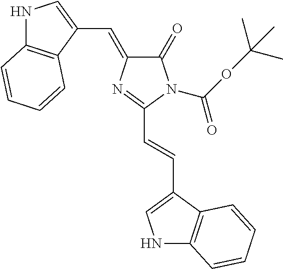 [SMILES: CC(C)(C)OC(=O)N1C(=O)/C(=C/c2c[nH]c3ccccc23)N=C1/C=C/c1c[nH]c2ccccc12] |
| 9 | 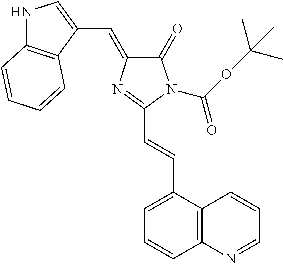 [SMILES: CC(C)(C)OC(=O)N1C(=O)/C(=C/c2c[nH]c3ccccc23)N=C1/C=C/c1cccc2ncccc12] |
| 10 | 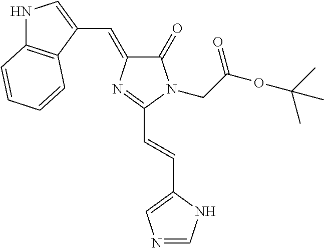 [SMILES: CC(C)(C)OC(=O)CN1C(=O)/C(=C/c2c[nH]c3ccccc23)N=C1/C=C/c1cnc[nH]1] |
| 11 | 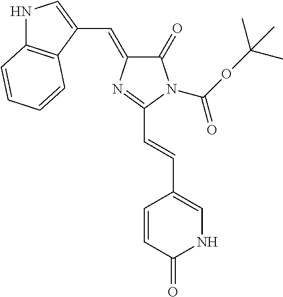 [SMILES: CC(C)(C)OC(=O)N1C(=O)/C(=C/c2c[nH]c3ccccc23)N=C1/C=C/c1ccc(=O)[nH]c1] |
| 12 | 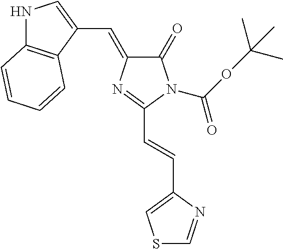 [SMILES: CC(C)(C)OC(=O)N1C(=O)/C(=C/c2c[nH]c3ccccc23)N=C1/C=C/c1cscn1] |
| 13 | 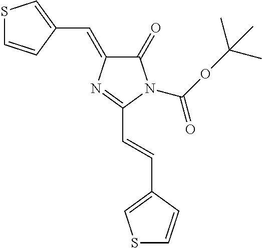 [SMILES: CC(C)(C)OC(=O)N1C(=O)/C(=C/c2ccsc2)N=C1/C=C/c1ccsc1] |
| 14 | 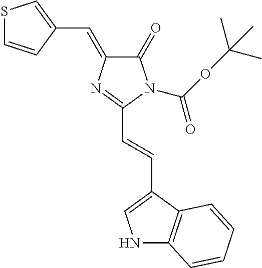 [SMILES: CC(C)(C)OC(=O)N1C(=O)/C(=C/c2ccsc2)N=C1/C=C/c1c[nH]c2ccccc12] |
| 15 | 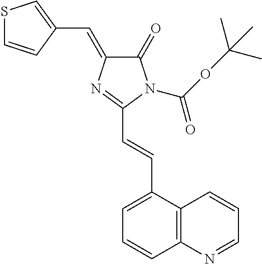 [SMILES: CC(C)(C)OC(=O)N1C(=O)/C(=C/c2ccsc2)N=C1/C=C/c1cccc2ncccc12] |
| 16 | 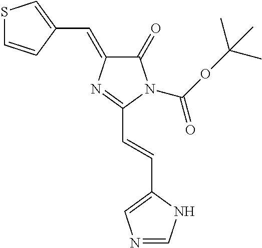 [SMILES: CC(C)(C)OC(=O)N1C(=O)/C(=C/c2ccsc2)N=C1/C=C/c1cnc[nH]1] |
| 17 | 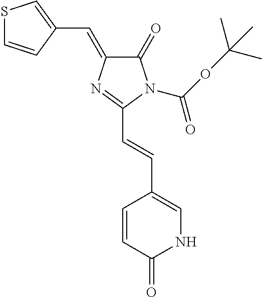 [SMILES: CC(C)(C)OC(=O)N1C(=O)/C(=C/c2ccsc2)N=C1/C=C/c1ccc(=O)[nH]c1] |
| 18 | 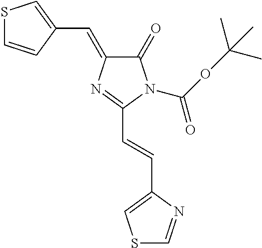 [SMILES: CC(C)(C)OC(=O)N1C(=O)/C(=C/c2ccsc2)N=C1/C=C/c1cscn1] |
| 19 | 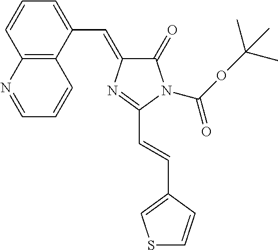 [SMILES: CC(C)(C)OC(=O)N1C(=O)/C(=C/c2cccc3ncccc23)N=C1/C=C/c1ccsc1] |
| 20 | 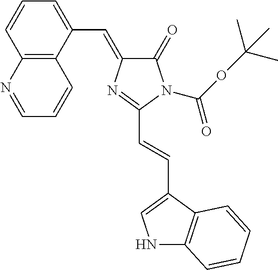 [SMILES: CC(C)(C)OC(=O)N1C(=O)/C(=C/c2cccc3ncccc23)N=C1/C=C/c1c[nH]c2ccccc12] |
| 21 | 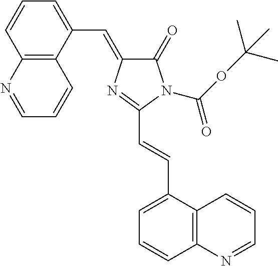 [SMILES: CC(C)(C)OC(=O)N1C(=O)/C(=C/c2cccc3ncccc23)N=C1/C=C/c1cccc2ncccc12] |
| 22 | 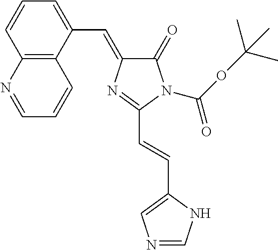 [SMILES: CC(C)(C)OC(=O)N1C(=O)/C(=C/c2cccc3ncccc23)N=C1/C=C/c1cnc[nH]1] |
| 23 | 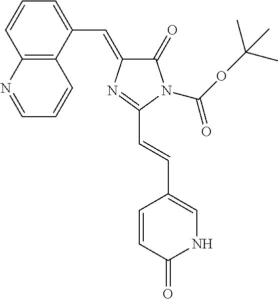 [SMILES: CC(C)(C)OC(=O)N1C(=O)/C(=C/c2cccc3ncccc23)N=C1/C=C/c1ccc(=O)[nH]c1] |
| 24 | 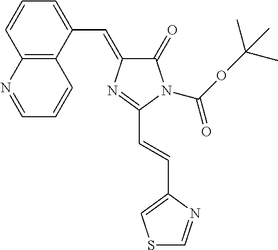 [SMILES: CC(C)(C)OC(=O)N1C(=O)/C(=C/c2cccc3ncccc23)N=C1/C=C/c1cscn1] |
| 25 | 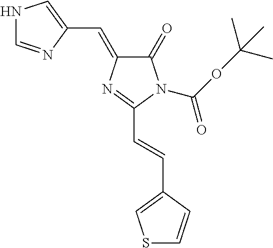 [SMILES: CC(C)(C)OC(=O)N1C(=O)/C(=C/c2c[nH]cn2)N=C1/C=C/c1ccsc1] |
| 26 | 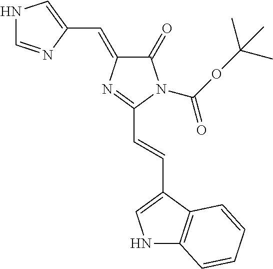 [SMILES: CC(C)(C)OC(=O)N1C(=O)/C(=C/c2c[nH]cn2)N=C1/C=C/c1c[nH]c2ccccc12] |
| 27 | 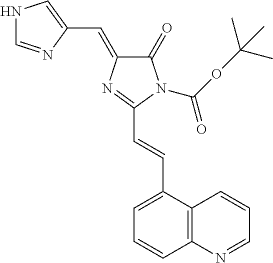 [SMILES: CC(C)(C)OC(=O)N1C(=O)/C(=C/c2c[nH]cn2)N=C1/C=C/c1cccc2ncccc12] |
| 28 | 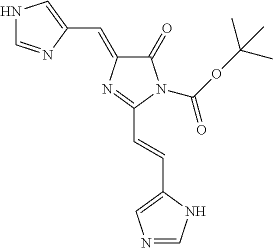 [SMILES: CC(C)(C)OC(=O)N1C(=O)/C(=C/c2c[nH]cn2)N=C1/C=C/c1cnc[nH]1] |
| 29 | 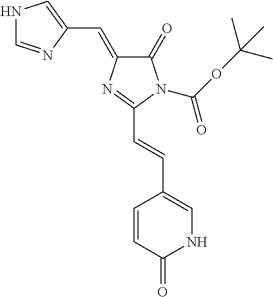 [SMILES: CC(C)(C)OC(=O)N1C(=O)/C(=C/c2c[nH]cn2)N=C1/C=C/c1ccc(=O)[nH]c1] |
| 30 | 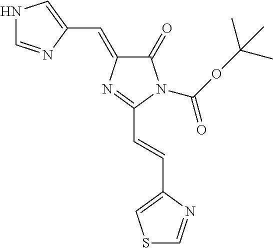 [SMILES: CC(C)(C)OC(=O)N1C(=O)/C(=C/c2c[nH]cn2)N=C1/C=C/c1cscn1] |
| 31 | 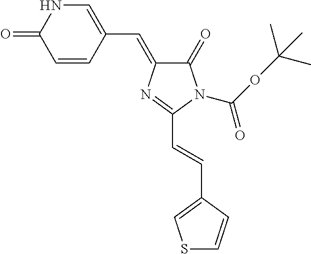 [SMILES: CC(C)(C)OC(=O)N1C(=O)/C(=C/c2ccc(=O)[nH]c2)N=C1/C=C/c1ccsc1] |
| 32 | 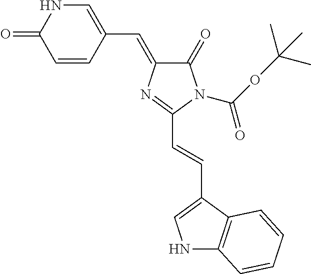 [SMILES: CC(C)(C)OC(=O)N1C(=O)/C(=C/c2ccc(=O)[nH]c2)N=C1/C=C/c1c[nH]c2ccccc12] |
| 33 | 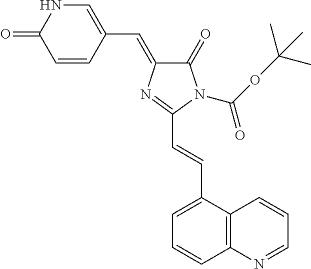 [SMILES: CC(C)(C)OC(=O)N1C(=O)/C(=C/c2ccc(=O)[nH]c2)N=C1/C=C/c1cccc2ncccc12] |
| 34 | 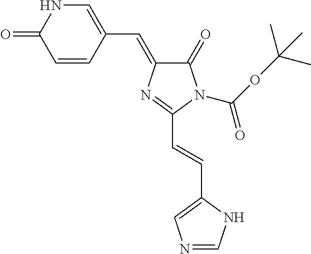 [SMILES: CC(C)(C)OC(=O)N1C(=O)/C(=C/c2ccc(=O)[nH]c2)N=C1/C=C/c1cnc[nH]1] |
| 35 | 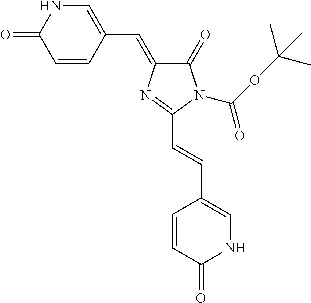 [SMILES: CC(C)(C)OC(=O)N1C(=O)/C(=C/c2ccc(=O)[nH]c2)N=C1/C=C/c1ccc(=O)[nH]c1] |
| 36 | 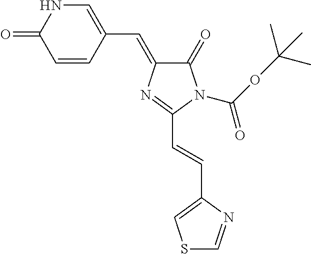 [SMILES: CC(C)(C)OC(=O)N1C(=O)/C(=C/c2ccc(=O)[nH]c2)N=C1/C=C/c1cscn1] |
| 37 | 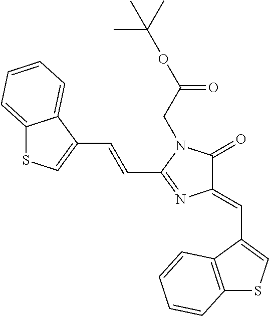 [SMILES: CC(C)(C)OC(=O)CN1C(=O)/C(=C/c2csc3ccccc23)N=C1/C=C/c1csc2ccccc12] |
| 38 | 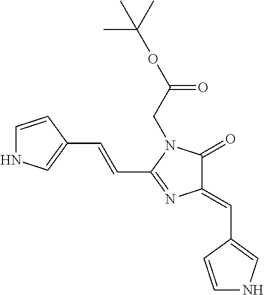 [SMILES: CC(C)(C)OC(=O)CN1C(=O)/C(=C/c2cc[nH]c2)N=C1/C=C/c1cc[nH]c1] |
| 39 | 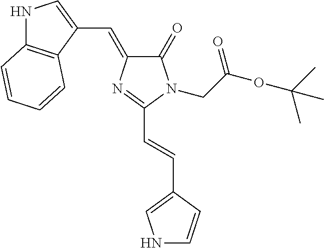 [SMILES: CC(C)(C)OC(=O)CN1C(=O)/C(=C/c2c[nH]c3ccccc23)N=C1/C=C/c1cc[nH]c1] |
| 40 | 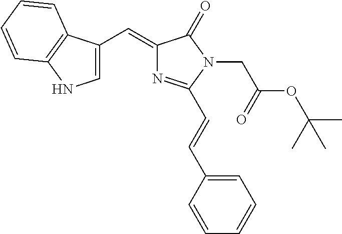 [SMILES: CC(C)(C)OC(=O)CN1C(=O)/C(=C/c2c[nH]c3ccccc23)N=C1/C=C/c1ccccc1] |
| 41 | 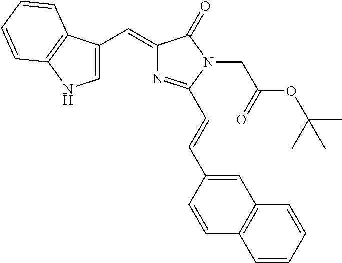 [SMILES: CC(C)(C)OC(=O)CN1C(=O)/C(=C/c2c[nH]c3ccccc23)N=C1/C=C/c1ccc2ccccc2c1] |
| 42 | 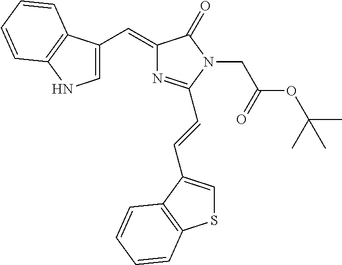 [SMILES: CC(C)(C)OC(=O)CN1C(=O)/C(=C/c2c[nH]c3ccccc23)N=C1/C=C/c1csc2ccccc12] |
| 43 | 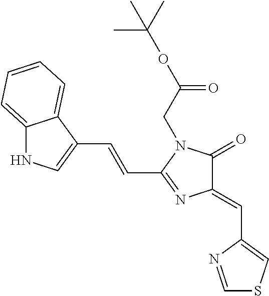 [SMILES: CC(C)(C)OC(=O)CN1C(=O)/C(=C/c2cscn2)N=C1/C=C/c1c[nH]c2ccccc12] |
| 44 | 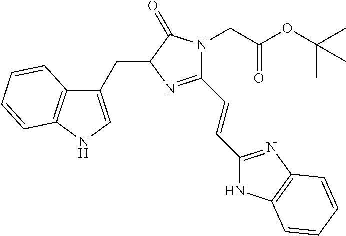 [SMILES: CC(C)(C)OC(=O)CN1C(=O)C(Cc2c[nH]c3ccccc23)N=C1/C=C/c1nc2ccccc2[nH]1] |
| 45 | 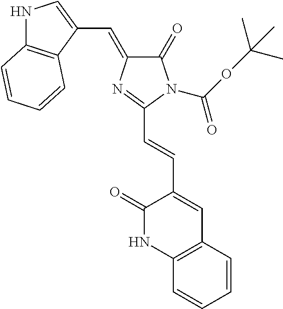 [SMILES: CC(C)(C)OC(=O)N1C(=O)/C(=C/c2c[nH]c3ccccc23)N=C1/C=C/c1cc2ccccc2[nH]c1=O] |
| 46 | 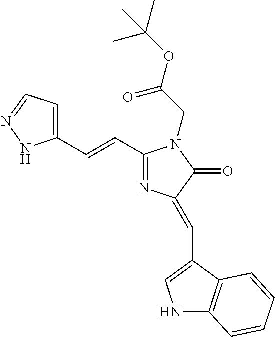 [SMILES: CC(C)(C)OC(=O)CN1C(=O)/C(=C/c2c[nH]c3ccccc23)N=C1/C=C/c1ccn[nH]1] |
| 47 | 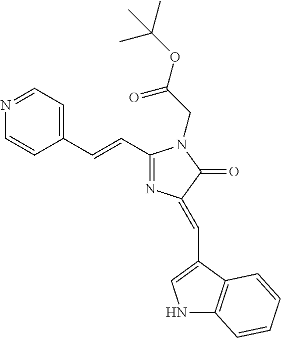 [SMILES: CC(C)(C)OC(=O)CN1C(=O)/C(=C/c2c[nH]c3ccccc23)N=C1/C=C/c1ccncc1] |
| 48 | 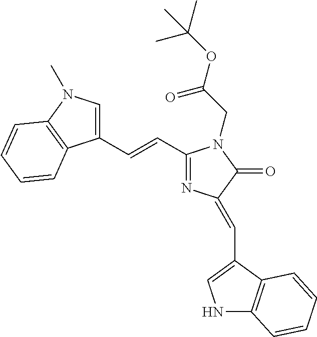 [SMILES: Cn1cc(/C=C/C2=NC(=C\c3c[nH]c4ccccc34)/C(=O)N2CC(=O)OC(C)(C)C)c2ccccc21] |
| 49 | 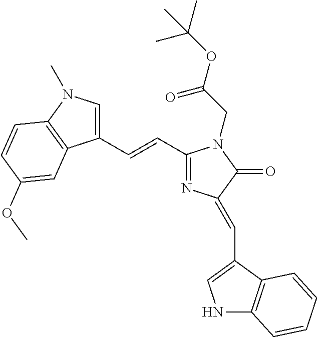 [SMILES: COc1ccc2c(c1)c(/C=C/C1=NC(=C\c3c[nH]c4ccccc34)/C(=O)N1CC(=O)OC(C)(C)C)cn2C] |
| 50 | 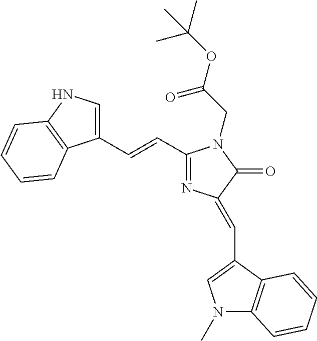 [SMILES: Cn1cc(/C=C2N=C(/C=C/c3c[nH]c4ccccc34)N(CC(=O)OC(C)(C)C)C\2=O)c2ccccc21] |
| 51 | 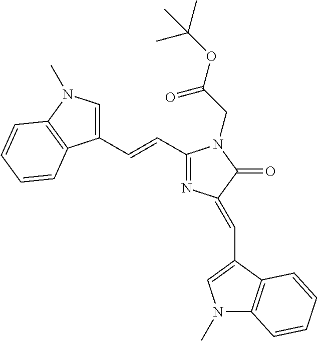 [SMILES: Cn1cc(/C=C2N=C(/C=C/c3cn(C)c4ccccc34)N(CC(=O)OC(C)(C)C)C\2=O)c2ccccc21] |
| 52 | 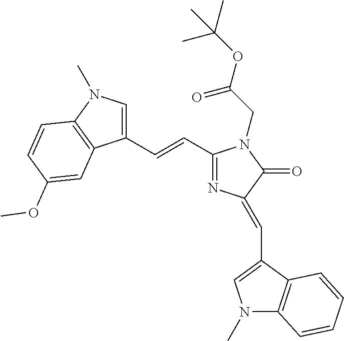 [SMILES: COc1ccc2c(c1)c(/C=C/C1=NC(=C\c3cn(C)c4ccccc34)/C(=O)N1CC(=O)OC(C)(C)C)cn2C] |
| 53 | 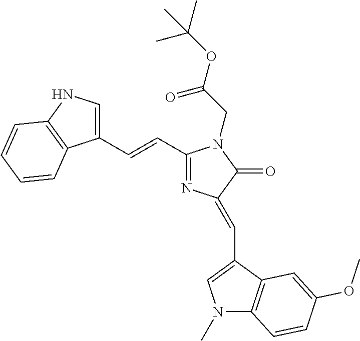 [SMILES: COc1ccc2c(c1)c(/C=C1N=C(/C=C/c3c[nH]c4ccccc34)N(CC(=O)OC(C)(C)C)C\1=O)cn2C] |
| 54 | 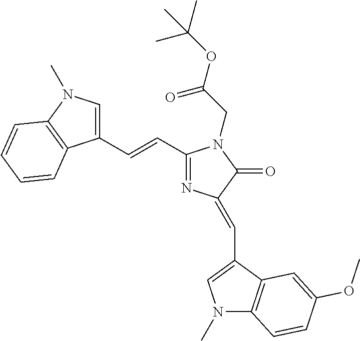 [SMILES: COc1ccc2c(c1)c(/C=C1N=C(/C=C/c3cn(C)c4ccccc34)N(CC(=O)OC(C)(C)C)C\1=O)cn2C] |
| 55 | 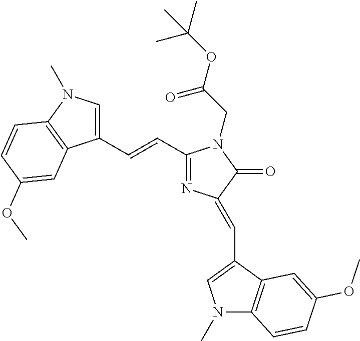 [SMILES: COc1ccc2c(c1)c(/C=C1N=C(/C=C/c3cn(C)c4ccc(OC)cc34)N(CC(=O)OC(C)(C)C)C\1=O)cn2C] |
| 56 | 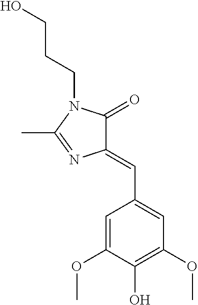 [SMILES: COc1cc(/C=C2\N=C(C)N(CCCO)C2=O)cc(OC)c1O] |
| 57 | 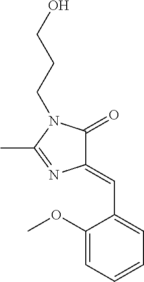 [SMILES: COc1ccccc1/C=C1\N=C(C)N(CCCO)C1=O] |
| 58 | 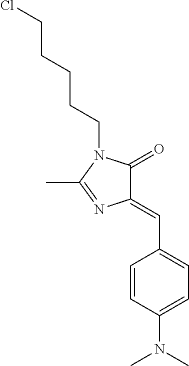 [SMILES: CC1=N/C(=C\c2ccc(N(C)C)cc2)C(=O)N1CCCCCCl] |
| 59 | 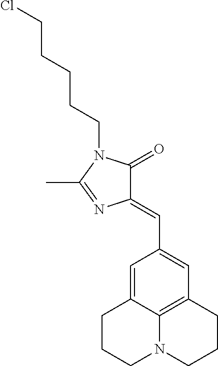 [SMILES: CC1=N/C(=C\c2cc3c4c(c2)CCCN4CCC3)C(=O)N1CCCCCCl] |
| 60 | 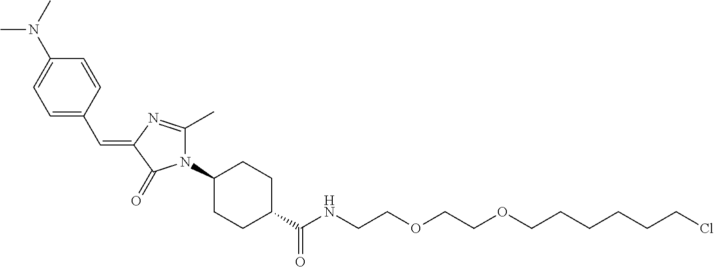 [SMILES: CC1=N/C(=C\c2ccc(N(C)C)cc2)C(=O)N1[C@H]1CC[C@H](C(=O)NCCOCCOCCCCCCCl)CC1] |
| 61 | 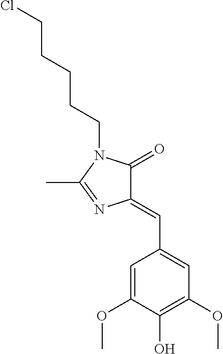 [SMILES: COc1cc(/C=C2\N=C(C)N(CCCCCCl)C2=O)cc(OC)c1O] |
| 62 | 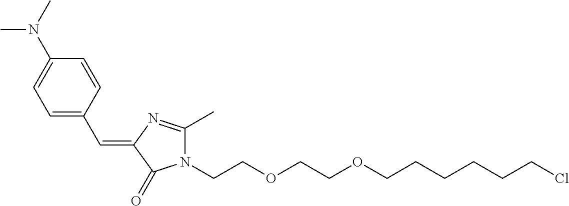 [SMILES: CC1=N/C(=C\c2ccc(N(C)C)cc2)C(=O)N1CCOCCOCCCCCCCl] |
| 63 | 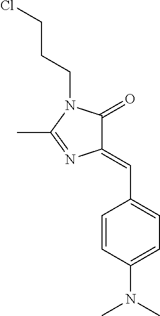 [SMILES: CC1=N/C(=C\c2ccc(N(C)C)cc2)C(=O)N1CCCCl] |
| 64 | 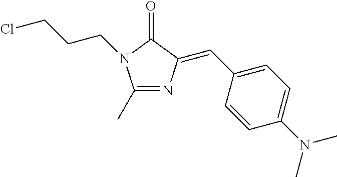 [SMILES: CC1=N/C(=C\c2ccc(N(C)C)cc2)C(=O)N1CCCCl] |
| 65 | 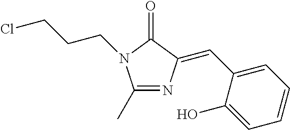 [SMILES: CC1=N/C(=C\c2ccccc2O)C(=O)N1CCCCl] |
| 66 | 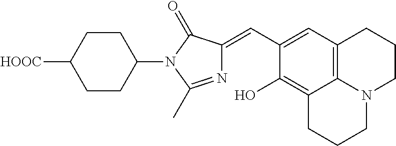 [SMILES: CC1=N/C(=C\c2cc3c4c(c2O)CCCN4CCC3)C(=O)N1C1CCC(C(=O)O)CC1] |
| 67 | 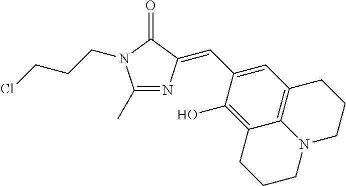 [SMILES: CC1=N/C(=C\c2cc3c4c(c2O)CCCN4CCC3)C(=O)N1CCCCl] |
| 68 | 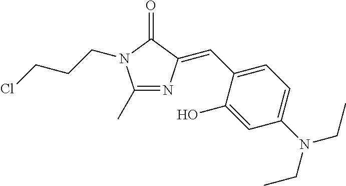 [SMILES: CCN(CC)c1ccc(/C=C2\N=C(C)N(CCCCl)C2=O)c(O)c1] |
| 69 | 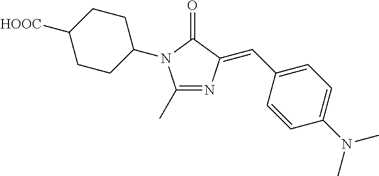 [SMILES: CC1=N/C(=C\c2ccc(N(C)C)cc2)C(=O)N1C1CCC(C(=O)O)CC1] |
| 70 | 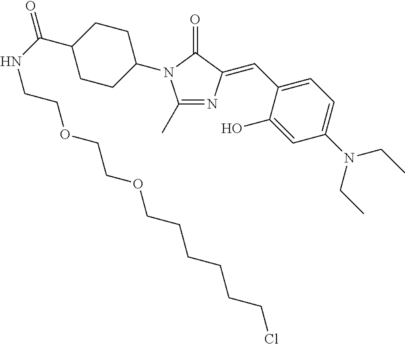 [SMILES: CCN(CC)c1ccc(/C=C2\N=C(C)N(C3CCC(C(=O)NCCOCCOCCCCCCCl)CC3)C2=O)c(O)c1] |
| 71 | 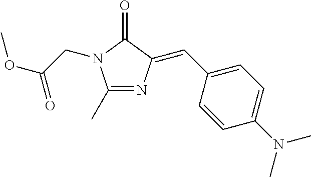 |
| 72 | 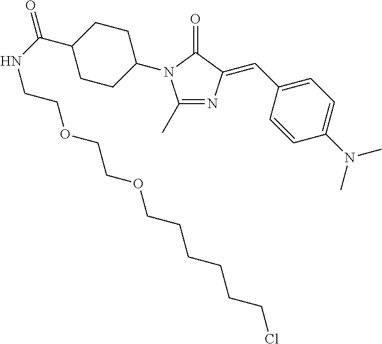 [SMILES: CC1=N/C(=C\c2ccc(N(C)C)cc2)C(=O)N1C1CCC(C(=O)NCCOCCOCCCCCCCl)CC1] |
| 73 | 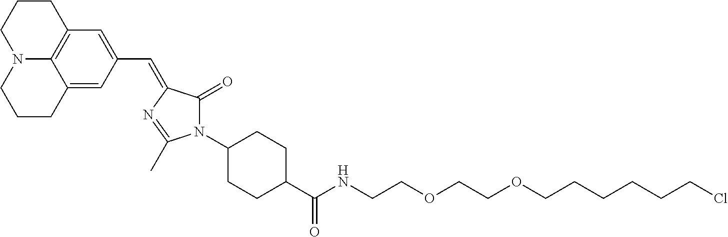 [SMILES: CC1=N/C(=C\c2cc3c4c(c2)CCCN4CCC3)C(=O)N1C1CCC(C(=O)NCCOCCOCCCCCCCl)CC1] |
| 74 | 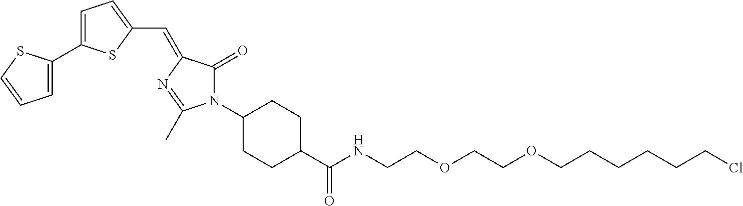 [SMILES: CC1=N/C(=C\c2ccc(-c3cccs3)s2)C(=O)N1C1CCC(C(=O)NCCOCCOCCCCCCCl)CC1] |
| 75 | 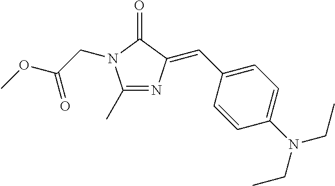 [SMILES: CCN(CC)c1ccc(/C=C2\N=C(C)N(CC(=O)OC)C2=O)cc1] |
| 76 | 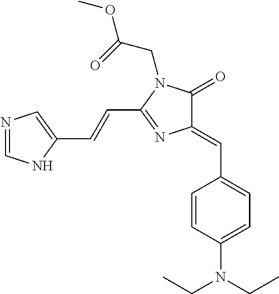 [SMILES: CCN(CC)c1ccc(/C=C2N=C(/C=C/c3cnc[nH]3)N(CC(=O)OC)C\2=O)cc1] |
| 77 | 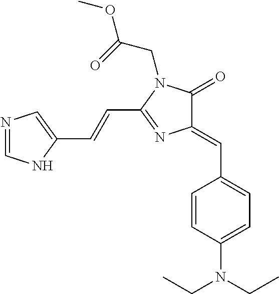 [SMILES: CCN(CC)c1ccc(/C=C2N=C(/C=C/c3cnc[nH]3)N(CC(=O)OC)C\2=O)cc1] |
| 78 | 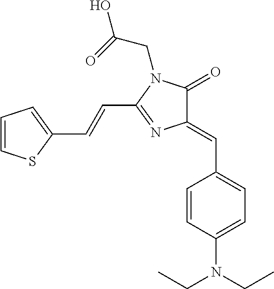 [SMILES: CCN(CC)c1ccc(/C=C2N=C(/C=C/c3cccs3)N(CC(=O)O)C\2=O)cc1] |
| 79 | 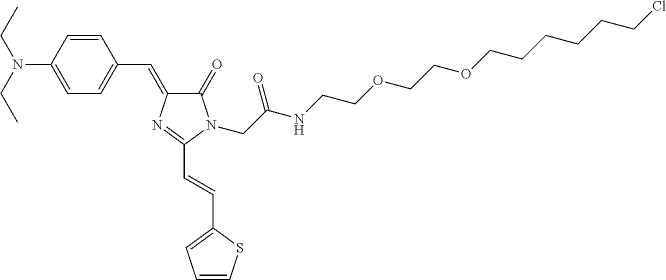 [SMILES: CCN(CC)c1ccc(/C=C2N=C(/C=C/c3cccs3)N(CC(=O)NCCOCCOCCCCCCCl)C\2=O)cc1] |
| 80 | 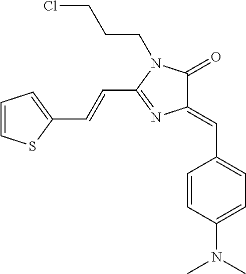 [SMILES: CN(C)c1ccc(/C=C2N=C(/C=C/c3cccs3)N(CCCCl)C\2=O)cc1] |
| 81 | 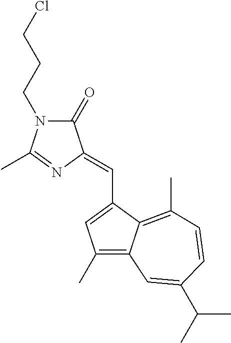 [SMILES: CC1=N/C(=C\c2cc(C)c3cc(C(C)C)ccc(C)c2-3)C(=O)N1CCCCl] |
| 82 | 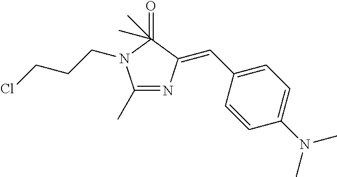 |
| 83 | 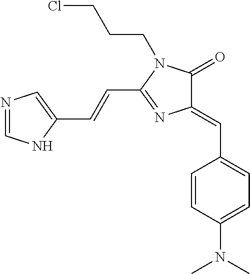 [SMILES: CN(C)c1ccc(/C=C2N=C(/C=C/c3cnc[nH]3)N(CCCCl)C\2=O)cc1] |
| 84 | 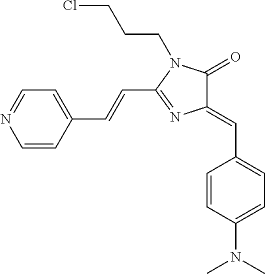 [SMILES: CN(C)c1ccc(/C=C2N=C(/C=C/c3ccncc3)N(CCCCl)C\2=O)cc1] |
| 85 | 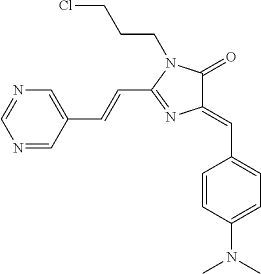 [SMILES: CN(C)c1ccc(/C=C2N=C(/C=C/c3cncnc3)N(CCCCl)C\2=O)cc1] |
| 86 | 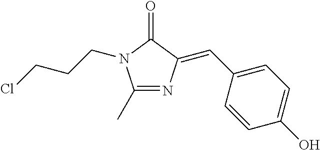 [SMILES: CC1=N/C(=C\c2ccc(O)cc2)C(=O)N1CCCCl] |
| 87 | 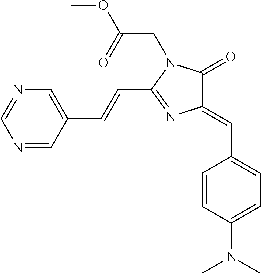 [SMILES: COC(=O)CN1C(=O)/C(=C/c2ccc(N(C)C)cc2)N=C1/C=C/c1cncnc1] |
| 88 | 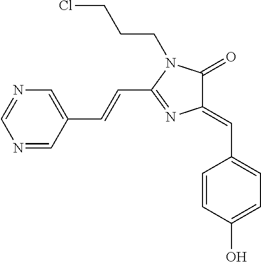 [SMILES: O=C1/C(=C/c2ccc(O)cc2)N=C(/C=C/c2cncnc2)N1CCCCl] |
| 89 | 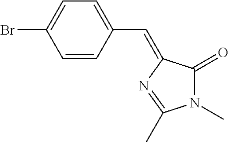 [SMILES: CC1=N/C(=C\c2ccc(Br)cc2)C(=O)N1C] |
| 90 | 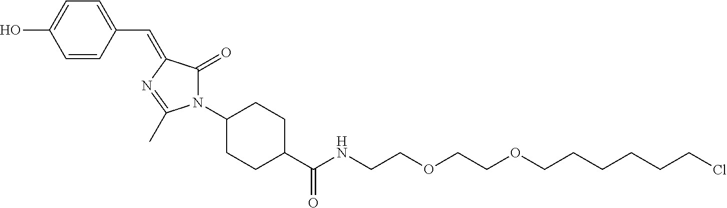 [SMILES: CC1=N/C(=C\c2ccc(O)cc2)C(=O)N1C1CCC(C(=O)NCCOCCOCCCCCCCl)CC1] |
| 91 | 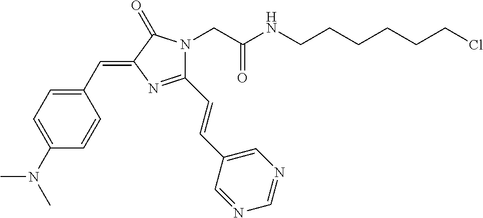 [SMILES: CN(C)c1ccc(/C=C2N=C(/C=C/c3cncnc3)N(CC(=O)NCCCCCCCl)C\2=O)cc1] |
| 92 | 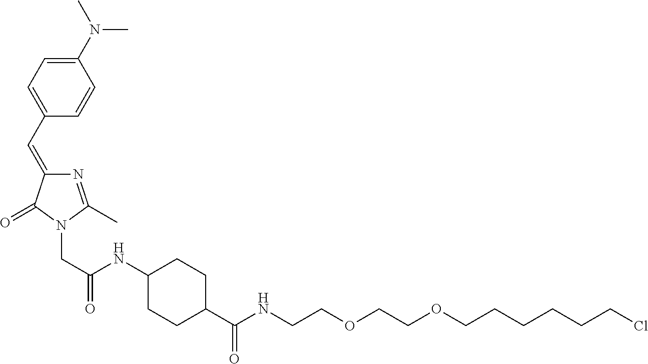 [SMILES: CC1=N/C(=C\c2ccc(N(C)C)cc2)C(=O)N1CC(=O)NC1CCC(C(=O)NCCOCCOCCCCCCCl)CC1] |
| 93 | 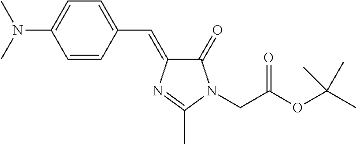 [SMILES: CC1=N/C(=C\c2ccc(N(C)C)cc2)C(=O)N1CC(=O)OC(C)(C)C] |
| 94 | 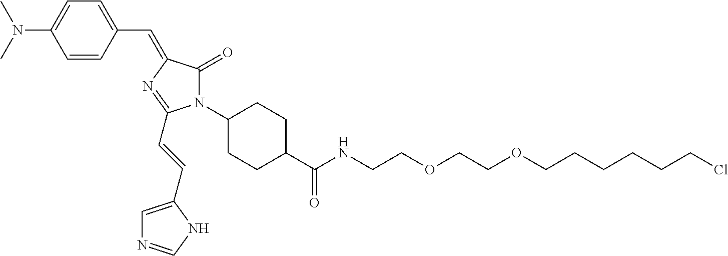 [SMILES: CN(C)c1ccc(/C=C2N=C(/C=C/c3cnc[nH]3)N(C3CCC(C(=O)NCCOCCOCCCCCCCl)CC3)C\2=O)cc1] |
| 95 | 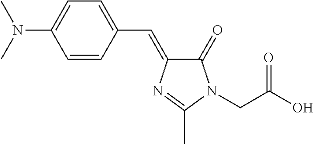 [SMILES: CC1=N/C(=C\c2ccc(N(C)C)cc2)C(=O)N1CC(=O)O] |
| 96 | 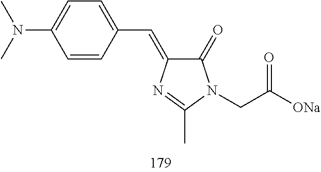 |
| 97 | 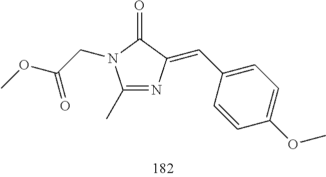 |
| 98 | 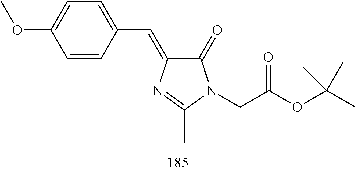 [SMILES: COc1ccc(/C=C2\N=C(C)N(CC(=O)OC(C)(C)C)C2=O)cc1] |
| 99 | 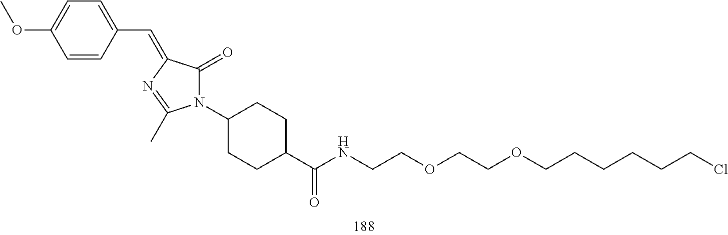 [SMILES: COc1ccc(/C=C2\N=C(C)N(C3CCC(C(=O)NCCOCCOCCCCCCCl)CC3)C2=O)cc1] |
| 100 | 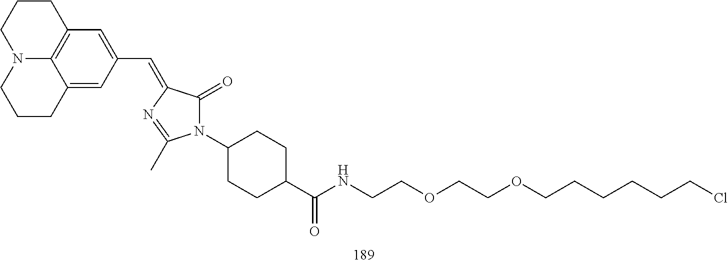 [SMILES: CC1=N/C(=C\c2cc3c4c(c2)CCCN4CCC3)C(=O)N1C1CCC(C(=O)NCCOCCOCCCCCCCl)CC1] |
| 101 | 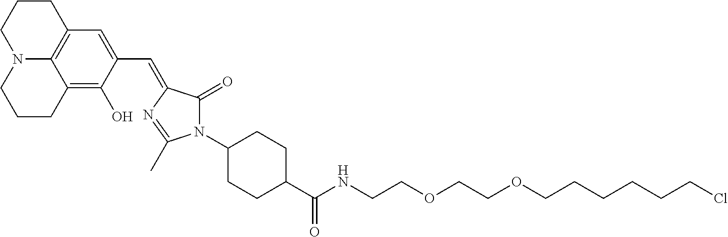 [SMILES: CC1=N/C(=C\c2cc3c4c(c2O)CCCN4CCC3)C(=O)N1C1CCC(C(=O)NCCOCCOCCCCCCCl)CC1] |
| 102 | 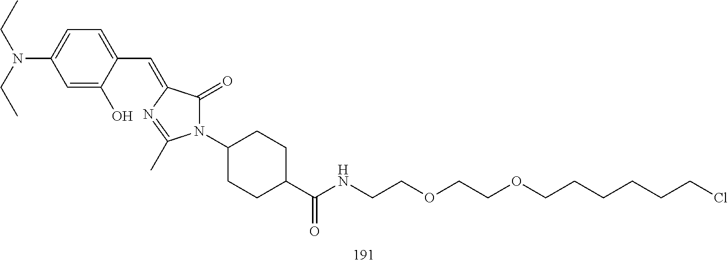 [SMILES: CCN(CC)c1ccc(/C=C2\N=C(C)N(C3CCC(C(=O)NCCOCCOCCCCCCCl)CC3)C2=O)c(O)c1] |
| 103 | 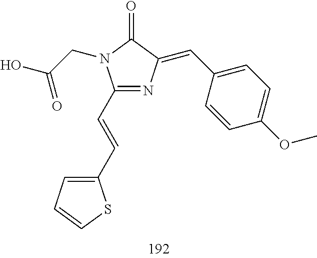 [SMILES: COc1ccc(/C=C2N=C(/C=C/c3cccs3)N(CC(=O)O)C\2=O)cc1] |
| 104 | 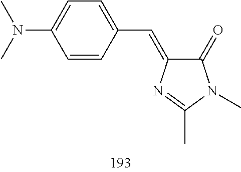 |
| 105 | 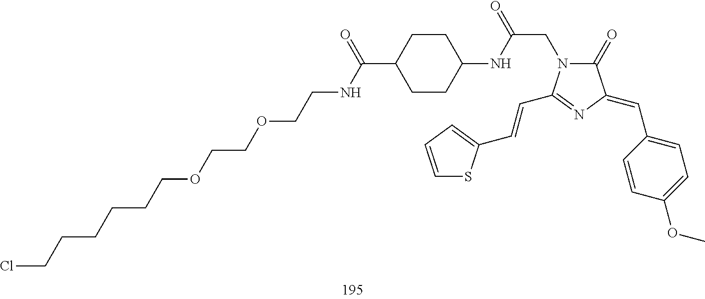 [SMILES: COc1ccc(/C=C2N=C(/C=C/c3cccs3)N(CC(=O)NC3CCC(C(=O)NCCOCCOCCCCCCCl)CC3)C\2=O)cc1] |
| 106 | 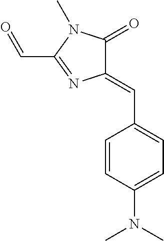 [SMILES: CN1C(=O)/C(=C/c2ccc(N(C)C)cc2)N=C1C=O] |
| 107 | 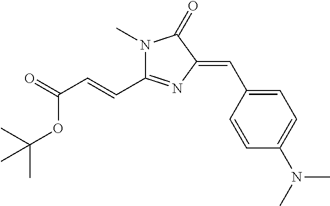 [SMILES: CN1C(=O)/C(=C/c2ccc(N(C)C)cc2)N=C1/C=C/C(=O)OC(C)(C)C] |
| 108 | 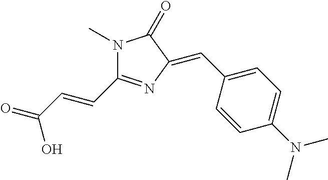 [SMILES: CN1C(=O)/C(=C/c2ccc(N(C)C)cc2)N=C1/C=C/C(=O)O] |
| 109 | 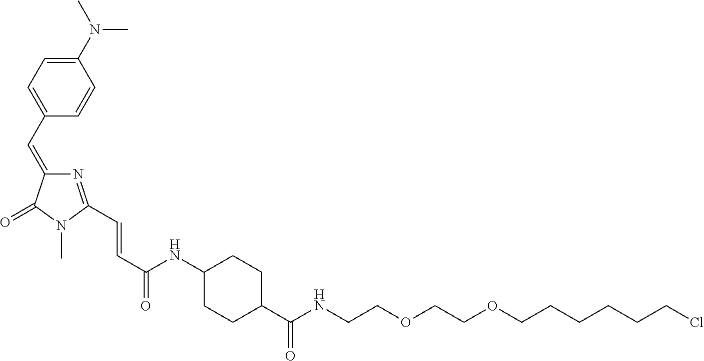 [SMILES: CN1C(=O)/C(=C/c2ccc(N(C)C)cc2)N=C1/C=C/C(=O)NC1CCC(C(=O)NCCOCCOCCCCCCCl)CC1] |
| 110 | 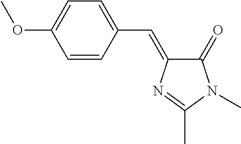 |
| 111 | 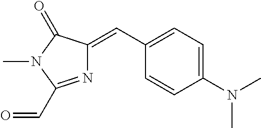 |
| 112 | 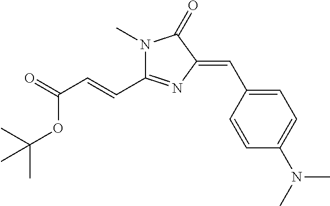 [SMILES: CN1C(=O)/C(=C/c2ccc(N(C)C)cc2)N=C1/C=C/C(=O)OC(C)(C)C] |
| 113 | 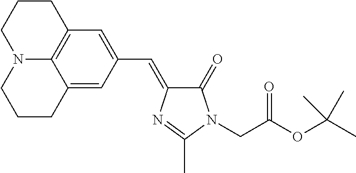 [SMILES: CC1=N/C(=C\c2cc3c4c(c2)CCCN4CCC3)C(=O)N1CC(=O)OC(C)(C)C] |
| 114 | 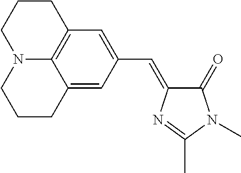 [SMILES: CC1=N/C(=C\c2cc3c4c(c2)CCCN4CCC3)C(=O)N1C] |
| 115 | 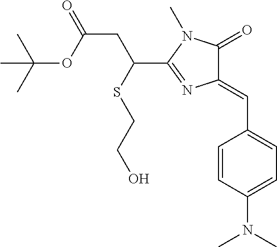 [SMILES: CN1C(=O)/C(=C/c2ccc(N(C)C)cc2)N=C1C(CC(=O)OC(C)(C)C)SCCO] |
| 116 | 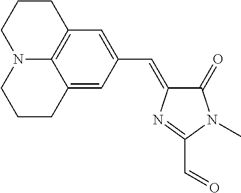 [SMILES: CN1C(=O)/C(=C/c2cc3c4c(c2)CCCN4CCC3)N=C1C=O] |
| 117 | 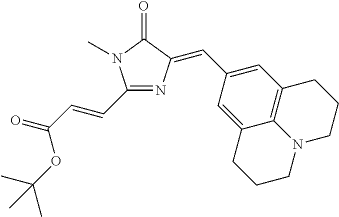 [SMILES: CN1C(=O)/C(=C/c2cc3c4c(c2)CCCN4CCC3)N=C1/C=C/C(=O)OC(C)(C)C] |
| 118 | 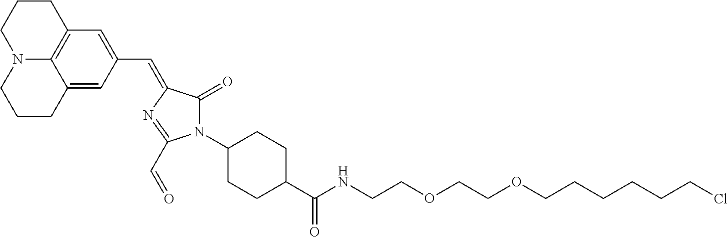 [SMILES: O=CC1=N/C(=C\c2cc3c4c(c2)CCCN4CCC3)C(=O)N1C1CCC(C(=O)NCCOCCOCCCCCCCl)CC1] |
| 119 | 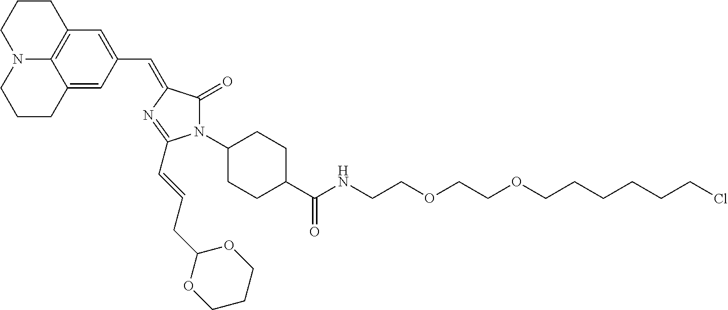 [SMILES: O=C(NCCOCCOCCCCCCCl)C1CCC(N2C(=O)/C(=C/c3cc4c5c(c3)CCCN5CCC4)N=C2/C=C/CC2OCCCO2)CC1] |
| 120 | 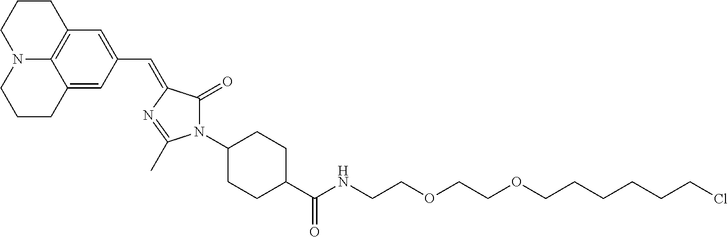 [SMILES: CC1=N/C(=C\c2cc3c4c(c2)CCCN4CCC3)C(=O)N1C1CCC(C(=O)NCCOCCOCCCCCCCl)CC1] |
| 121 | 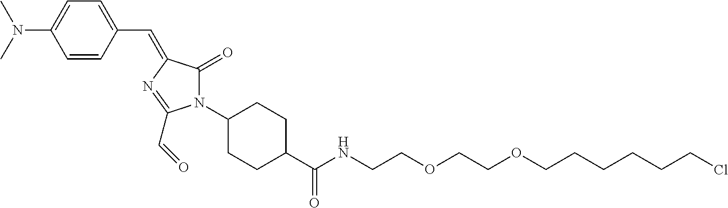 [SMILES: CN(C)c1ccc(/C=C2\N=C(C=O)N(C3CCC(C(=O)NCCOCCOCCCCCCCl)CC3)C2=O)cc1] |
| 122 | 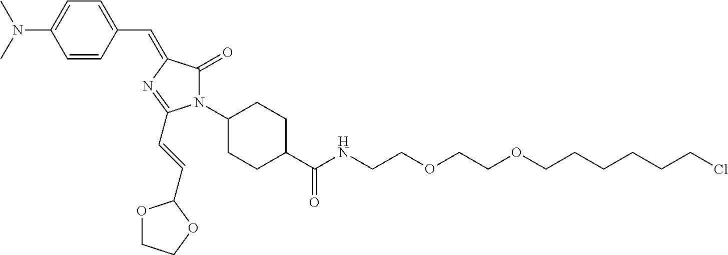 [SMILES: CN(C)c1ccc(/C=C2N=C(/C=C/C3OCCO3)N(C3CCC(C(=O)NCCOCCOCCCCCCCl)CC3)C\2=O)cc1] |
| 123 | 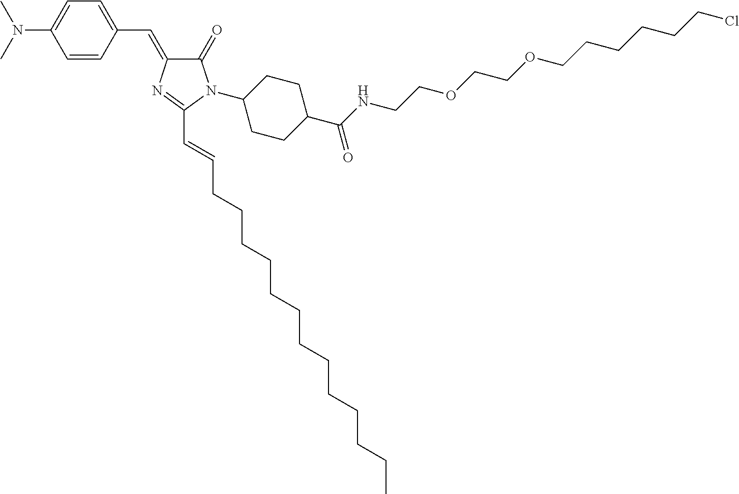 [SMILES: CCCCCCCCCCCCC/C=C/C1=NC(=C\c2ccc(N(C)C)cc2)/C(=O)N1C1CCC(C(=O)NCCOCCOCCCCCCCl)CC1] |
| 124 | 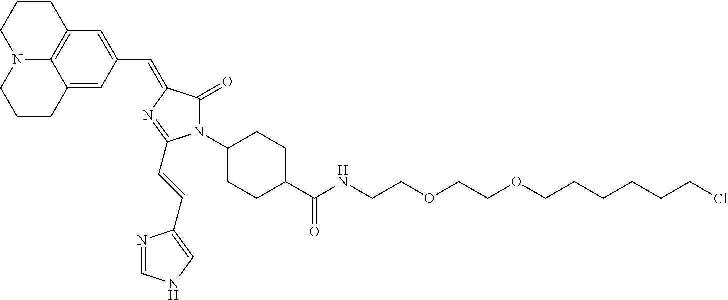 [SMILES: O=C(NCCOCCOCCCCCCCl)C1CCC(N2C(=O)/C(=C/c3cc4c5c(c3)CCCN5CCC4)N=C2/C=C/c2c[nH]cn2)CC1] |
| 125 | 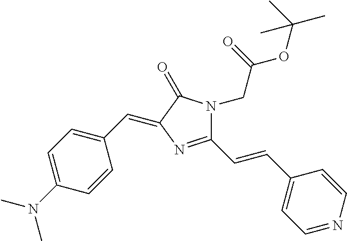 [SMILES: CN(C)c1ccc(/C=C2N=C(/C=C/c3ccncc3)N(CC(=O)OC(C)(C)C)C\2=O)cc1] |
| 126 | 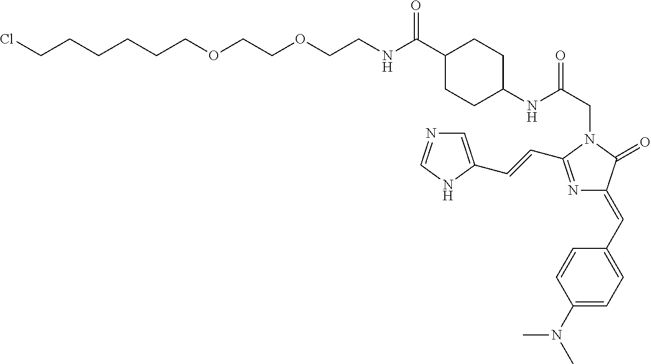 [SMILES: CN(C)c1ccc(/C=C2N=C(/C=C/c3cnc[nH]3)N(CC(=O)NC3CCC(C(=O)NCCOCCOCCCCCCCl)CC3)C\2=O)cc1] |
| 127 | 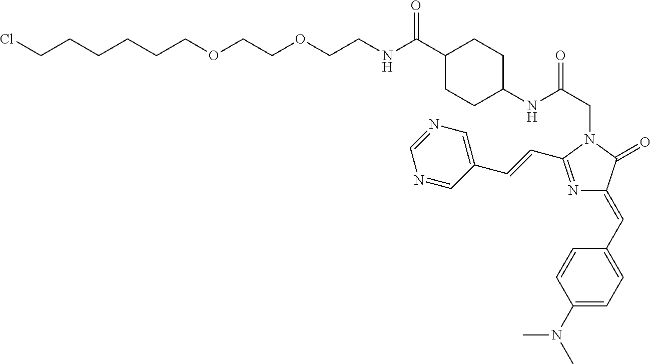 [SMILES: CN(C)c1ccc(/C=C2N=C(/C=C/c3cncnc3)N(CC(=O)NC3CCC(C(=O)NCCOCCOCCCCCCCl)CC3)C\2=O)cc1] |
| 128 | 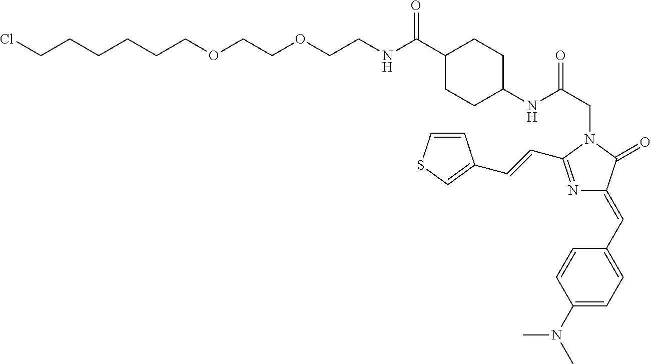 [SMILES: CN(C)c1ccc(/C=C2N=C(/C=C/c3ccsc3)N(CC(=O)NC3CCC(C(=O)NCCOCCOCCCCCCCl)CC3)C\2=O)cc1] |
| 129 | 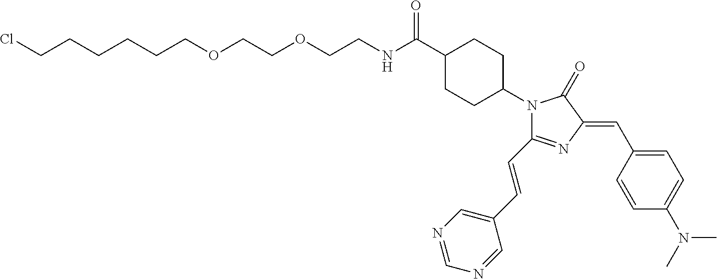 [SMILES: CN(C)c1ccc(/C=C2N=C(/C=C/c3cncnc3)N(C3CCC(C(=O)NCCOCCOCCCCCCCl)CC3)C\2=O)cc1] |
| 130 | 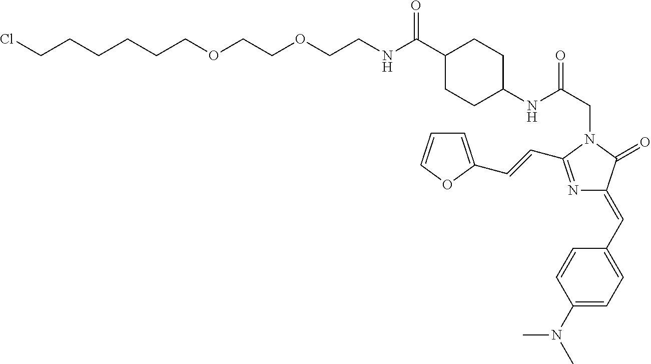 [SMILES: CN(C)c1ccc(/C=C2N=C(/C=C/c3ccco3)N(CC(=O)NC3CCC(C(=O)NCCOCCOCCCCCCCl)CC3)C\2=O)cc1] |
| 131 | 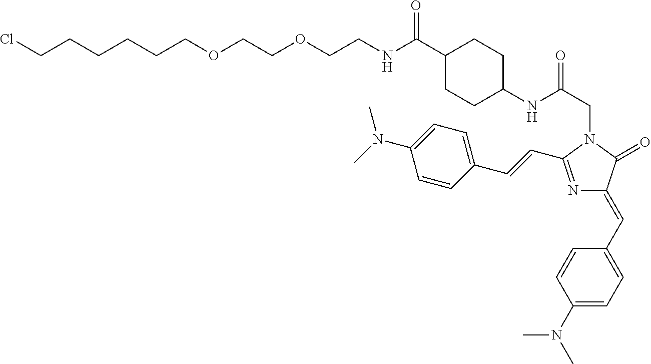 [SMILES: CN(C)c1ccc(/C=C2N=C(/C=C/c3ccc(N(C)C)cc3)N(CC(=O)NC3CCC(C(=O)NCCOCCOCCCCCCCl)CC3)C\2=O)cc1] |
| 132 | 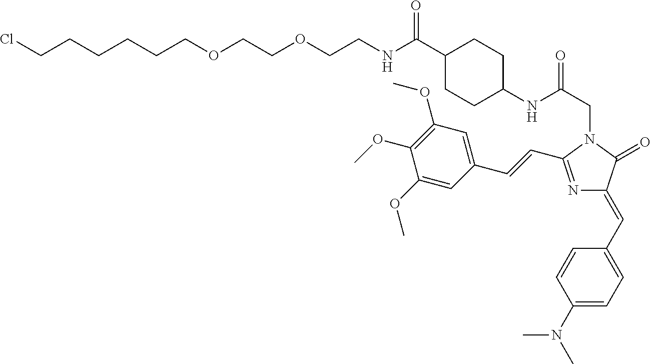 [SMILES: COc1cc(/C=C/C2=NC(=C\c3ccc(N(C)C)cc3)/C(=O)N2CC(=O)NC2CCC(C(=O)NCCOCCOCCCCCCCl)CC2)cc(OC)c1OC] |
| 133 | 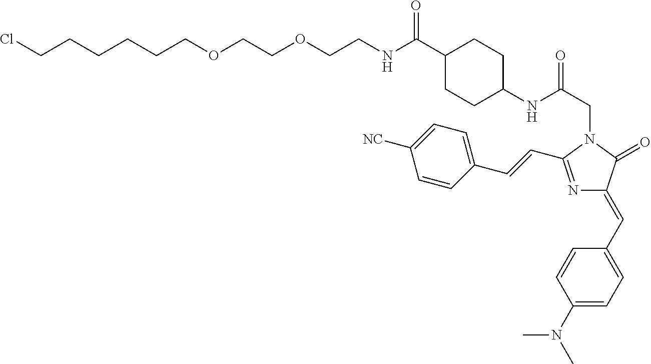 [SMILES: CN(C)c1ccc(/C=C2N=C(/C=C/c3ccc(C#N)cc3)N(CC(=O)NC3CCC(C(=O)NCCOCCOCCCCCCCl)CC3)C\2=O)cc1] |
| 134 | 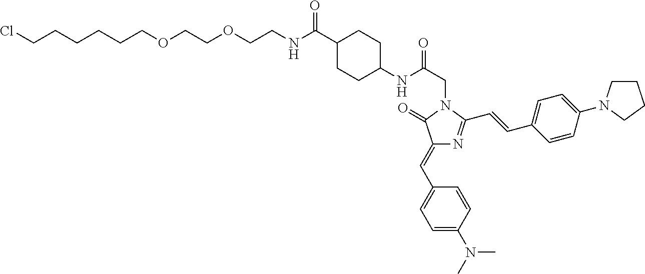 [SMILES: CN(C)c1ccc(/C=C2N=C(/C=C/c3ccc(N4CCCC4)cc3)N(CC(=O)NC3CCC(C(=O)NCCOCCOCCCCCCCl)CC3)C\2=O)cc1] |
| 135 | 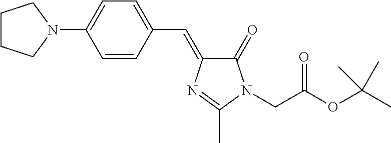 [SMILES: CC1=N/C(=C\c2ccc(N3CCCC3)cc2)C(=O)N1CC(=O)OC(C)(C)C] |
| 136 | 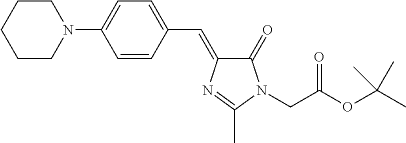 [SMILES: CC1=N/C(=C\c2ccc(N3CCCCC3)cc2)C(=O)N1CC(=O)OC(C)(C)C] |
| 137 | 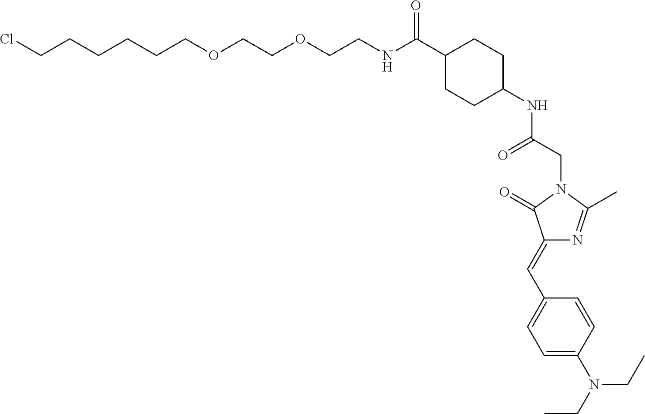 [SMILES: CCN(CC)c1ccc(/C=C2\N=C(C)N(CC(=O)NC3CCC(C(=O)NCCOCCOCCCCCCCl)CC3)C2=O)cc1] |
| 138 | 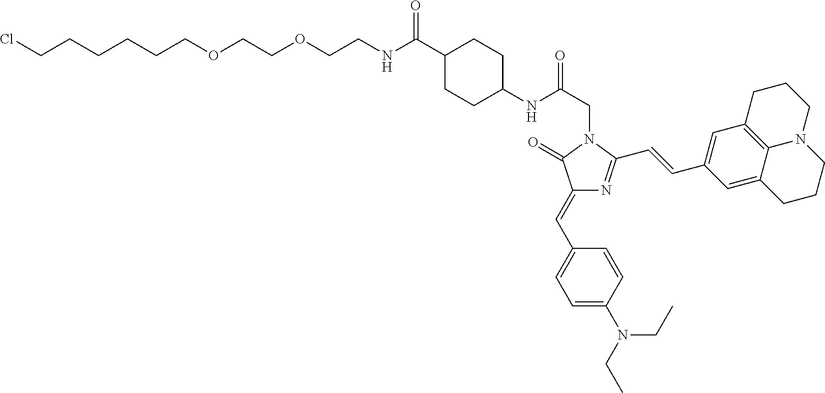 [SMILES: CCN(CC)c1ccc(/C=C2N=C(/C=C/c3cc4c5c(c3)CCCN5CCC4)N(CC(=O)NC3CCC(C(=O)NCCOCCOCCCCCCCl)CC3)C\2=O)cc1] |
| 139 | 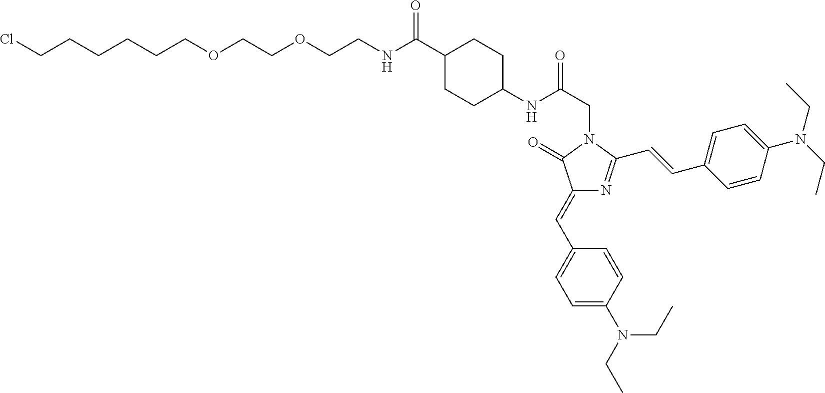 [SMILES: CCN(CC)c1ccc(/C=C2N=C(/C=C/c3ccc(N(CC)CC)cc3)N(CC(=O)NC3CCC(C(=O)NCCOCCOCCCCCCCl)CC3)C\2=O)cc1] |
| 140 | 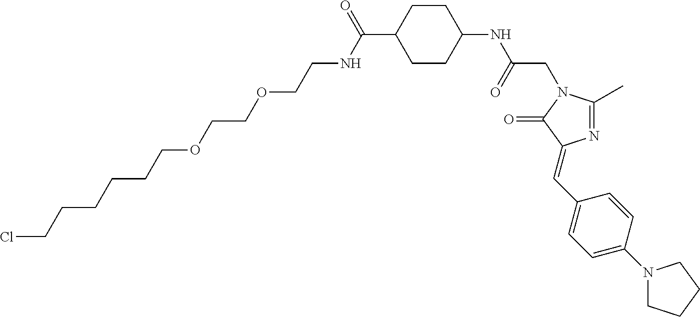 [SMILES: CC1=N/C(=C\c2ccc(N3CCCC3)cc2)C(=O)N1CC(=O)NC1CCC(C(=O)NCCOCCOCCCCCCCl)CC1] |
| 141 | 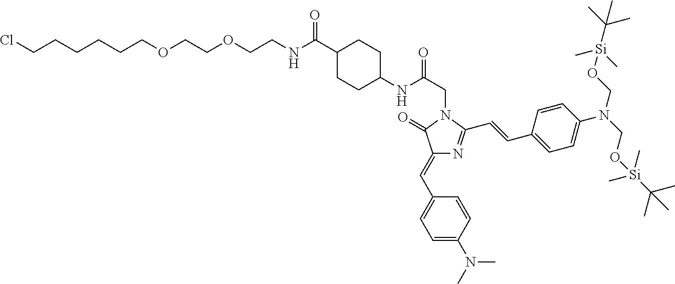 [SMILES: CN(C)c1ccc(/C=C2N=C(/C=C/c3ccc(N(CO[Si](C)(C)C(C)(C)C)CO[Si](C)(C)C(C)(C)C)cc3)N(CC(=O)NC3CCC(C(=O)NCCOCCOCCCCCCCl)CC3)C\2=O)cc1] |
| 142 | 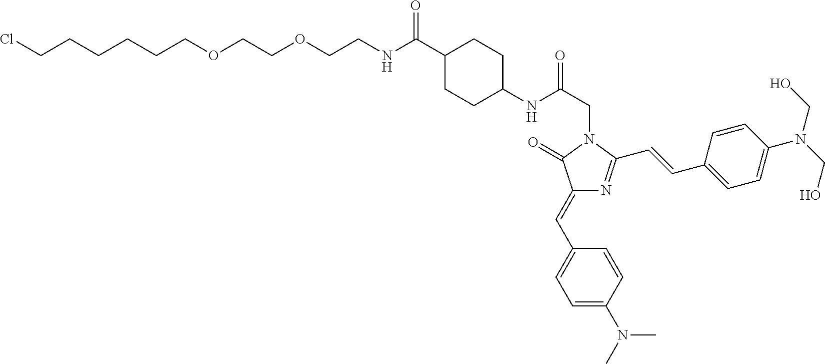 [SMILES: CN(C)c1ccc(/C=C2N=C(/C=C/c3ccc(N(CO)CO)cc3)N(CC(=O)NC3CCC(C(=O)NCCOCCOCCCCCCCl)CC3)C\2=O)cc1] |
| 143 | 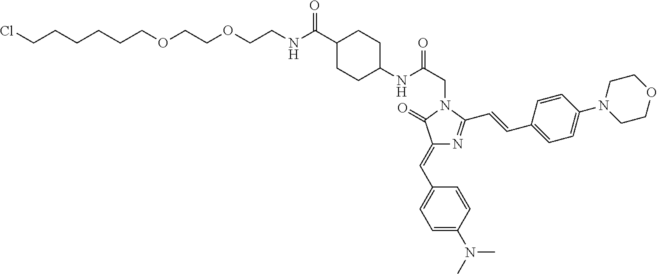 [SMILES: CN(C)c1ccc(/C=C2N=C(/C=C/c3ccc(N4CCOCC4)cc3)N(CC(=O)NC3CCC(C(=O)NCCOCCOCCCCCCCl)CC3)C\2=O)cc1] |
| 144 | 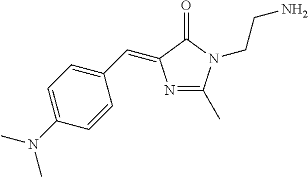 [SMILES: CC1=N/C(=C\c2ccc(N(C)C)cc2)C(=O)N1CCN] |
| 145 | 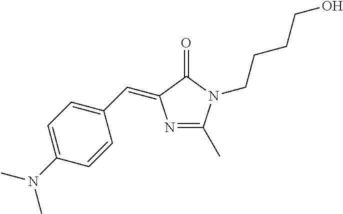 [SMILES: CC1=N/C(=C\c2ccc(N(C)C)cc2)C(=O)N1CCCCO] |
| 146 | 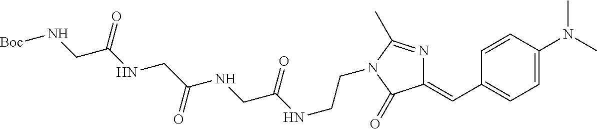 [SMILES: CC1=N/C(=C\c2ccc(N(C)C)cc2)C(=O)N1CCNC(=O)CNC(=O)CNC(=O)CNC(=O)OC(C)(C)C] |
| 147 | 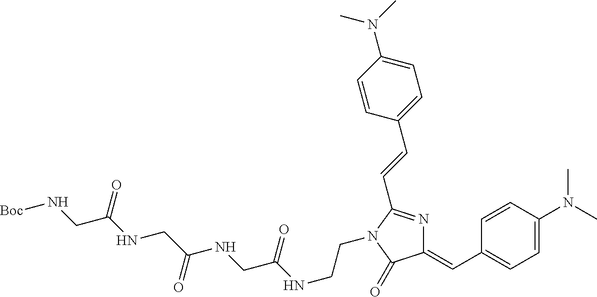 [SMILES: CN(C)c1ccc(/C=C2N=C(/C=C/c3ccc(N(C)C)cc3)N(CCNC(=O)CNC(=O)CNC(=O)CNC(=O)OC(C)(C)C)C\2=O)cc1] |
| 148 | 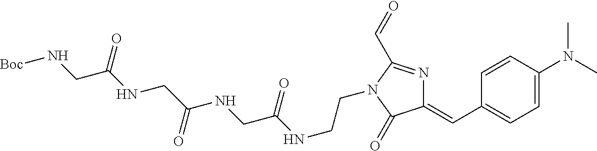 [SMILES: CN(C)c1ccc(/C=C2\N=C(C=O)N(CCNC(=O)CNC(=O)CNC(=O)CNC(=O)OC(C)(C)C)C2=O)cc1] |
| 149 | 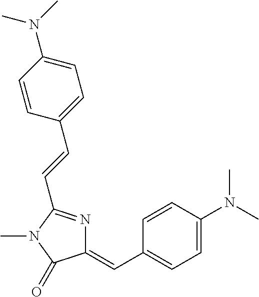 |
| 150 | 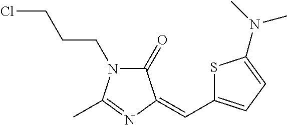 [SMILES: CC1=N/C(=C/c2ccc(N(C)C)s2)C(=O)N1CCCCl] |
| 151 | 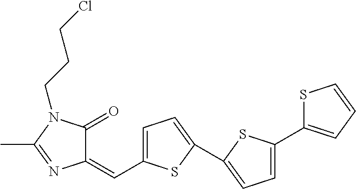 [SMILES: CC1=N/C(=C/c2ccc(-c3ccc(-c4cccs4)s3)s2)C(=O)N1CCCCl] |
| 152 | 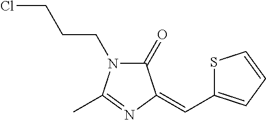 [SMILES: CC1=N/C(=C/c2cccs2)C(=O)N1CCCCl] |
| 153 | 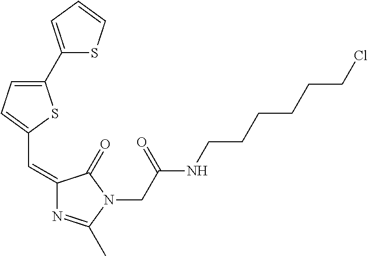 [SMILES: CC1=N/C(=C/c2ccc(-c3cccs3)s2)C(=O)N1CC(=O)NCCCCCCCl] |
| 154 | 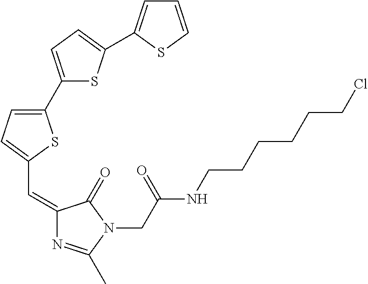 [SMILES: CC1=N/C(=C/c2ccc(-c3ccc(-c4cccs4)s3)s2)C(=O)N1CC(=O)NCCCCCCCl] |
| 155 | 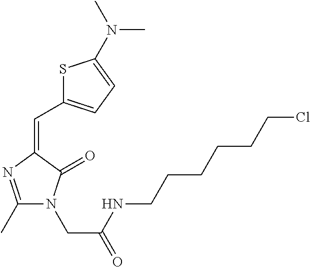 [SMILES: CC1=N/C(=C/c2ccc(N(C)C)s2)C(=O)N1CC(=O)NCCCCCCCl] |
| 156 | 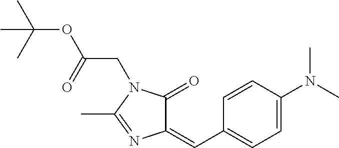 [SMILES: CC1=N/C(=C/c2ccc(N(C)C)cc2)C(=O)N1CC(=O)OC(C)(C)C] |
| 157 | 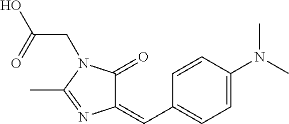 [SMILES: CC1=N/C(=C/c2ccc(N(C)C)cc2)C(=O)N1CC(=O)O] |
| 158 | 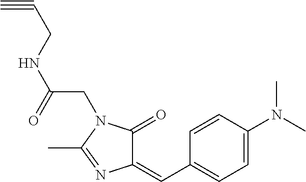 [SMILES: C#CCNC(=O)CN1C(=O)/C(=C\c2ccc(N(C)C)cc2)N=C1C] |
| 159 | 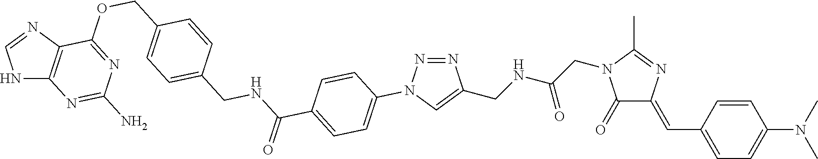 [SMILES: CC1=N/C(=C\c2ccc(N(C)C)cc2)C(=O)N1CC(=O)NCc1cn(-c2ccc(C(=O)NCc3ccc(COc4nc(N)nc5[nH]cnc45)cc3)cc2)nn1] |
| 160 | 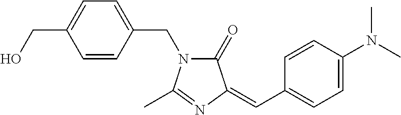 [SMILES: CC1=N/C(=C/c2ccc(N(C)C)cc2)C(=O)N1Cc1ccc(CO)cc1] |
| 161 | 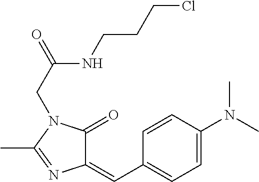 |
| 162 | 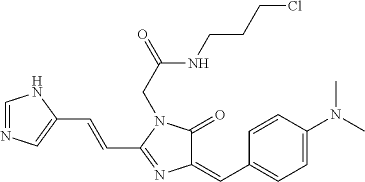 [SMILES: CN(C)c1ccc(/C=C2N=C(/C=C/c3cnc[nH]3)N(CC(=O)NCCCCl)C/2=O)cc1] |
| 163 | 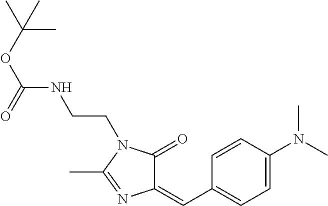 [SMILES: CC1=N/C(=C/c2ccc(N(C)C)cc2)C(=O)N1CCNC(=O)OC(C)(C)C] |
| 164 | 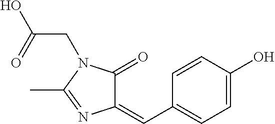 [SMILES: CC1=N/C(=C/c2ccc(O)cc2)C(=O)N1CC(=O)O] |
| 165 | 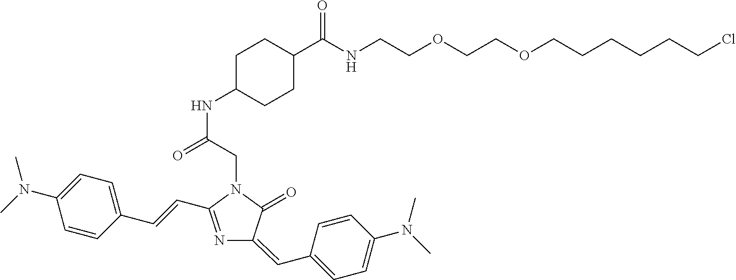 [SMILES: CN(C)c1ccc(/C=C/C2=NC(=C/c3ccc(N(C)C)cc3)/C(=O)N2CC(=O)NC2CCC(C(=O)NCCOCCOCCCCCCCl)CC2)cc1] |
| 166 | 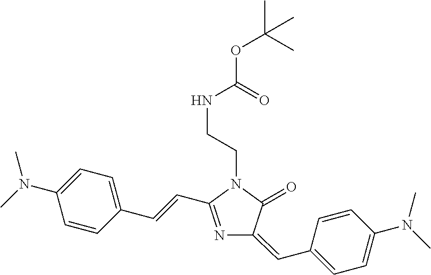 |
| 167 | 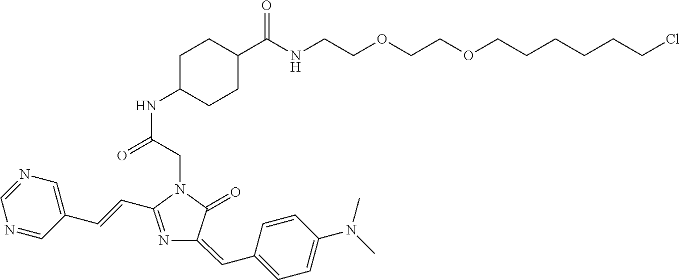 [SMILES: CN(C)c1ccc(/C=C2N=C(/C=C/c3cncnc3)N(CC(=O)NC3CCC(C(=O)NCCOCCOCCCCCCCl)CC3)C/2=O)cc1] |
| 168 | 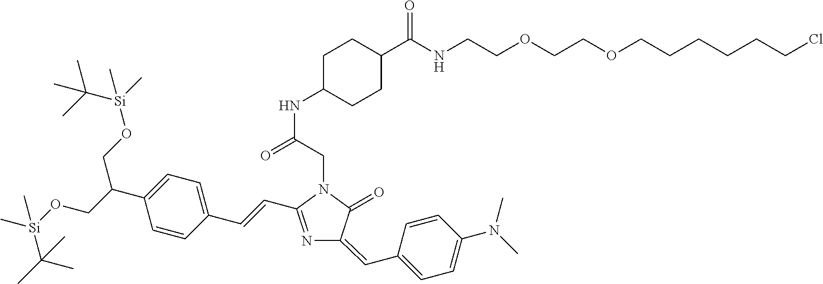 [SMILES: CN(C)c1ccc(/C=C2N=C(/C=C/c3ccc(C(CO[Si](C)(C)C(C)(C)C)CO[Si](C)(C)C(C)(C)C)cc3)N(CC(=O)NC3CCC(C(=O)NCCOCCOCCCCCCCl)CC3)C/2=O)cc1] |
| 169 | 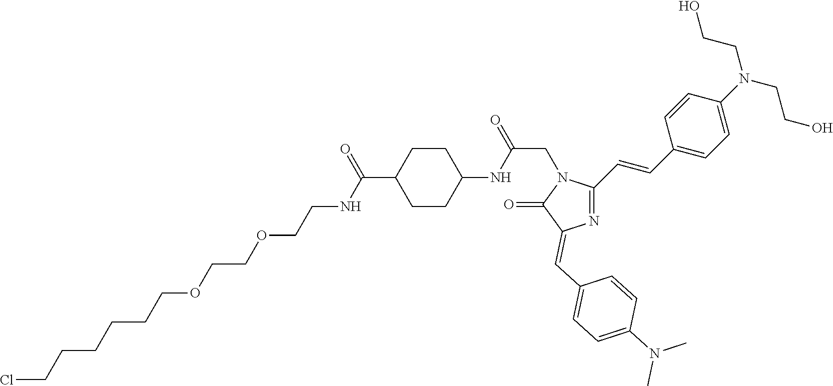 [SMILES: CN(C)c1ccc(/C=C2N=C(/C=C/c3ccc(N(CCO)CCO)cc3)N(CC(=O)NC3CCC(C(=O)NCCOCCOCCCCCCCl)CC3)C\2=O)cc1] |
| 170 | 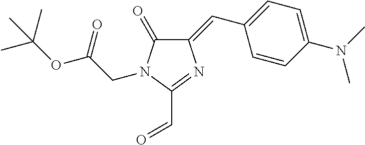 [SMILES: CN(C)c1ccc(/C=C2\N=C(C=O)N(CC(=O)OC(C)(C)C)C2=O)cc1] |
| 171 | 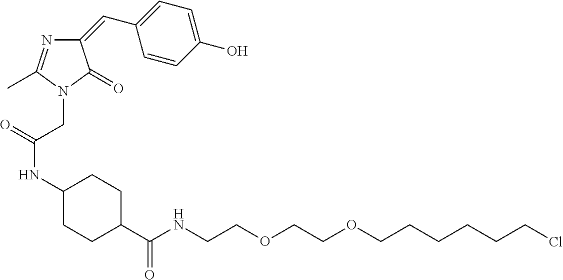 [SMILES: CC1=N/C(=C/c2ccc(O)cc2)C(=O)N1CC(=O)NC1CCC(C(=O)NCCOCCOCCCCCCCl)CC1] |
| 172 | 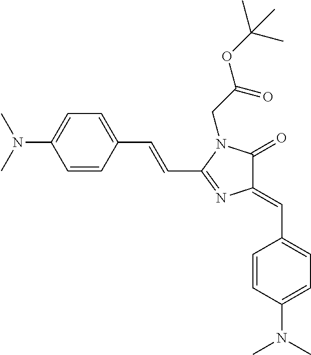 |
| 173 | 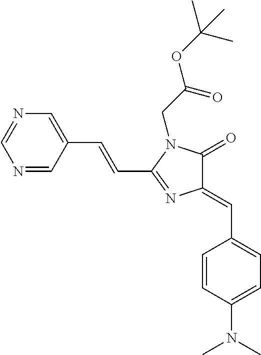 [SMILES: CN(C)c1ccc(/C=C2N=C(/C=C/c3cncnc3)N(CC(=O)OC(C)(C)C)C\2=O)cc1] |
| 174 | 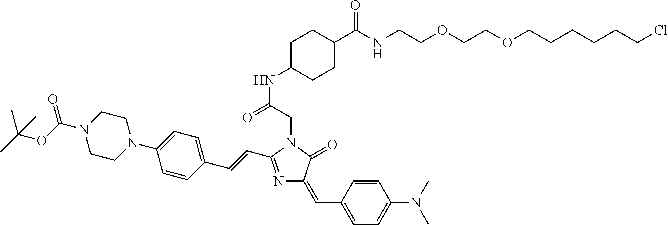 [SMILES: CN(C)c1ccc(/C=C2N=C(/C=C/c3ccc(N4CCN(C(=O)OC(C)(C)C)CC4)cc3)N(CC(=O)NC3CCC(C(=O)NCCOCCOCCCCCCCl)CC3)C/2=O)cc1] |
| 175 | 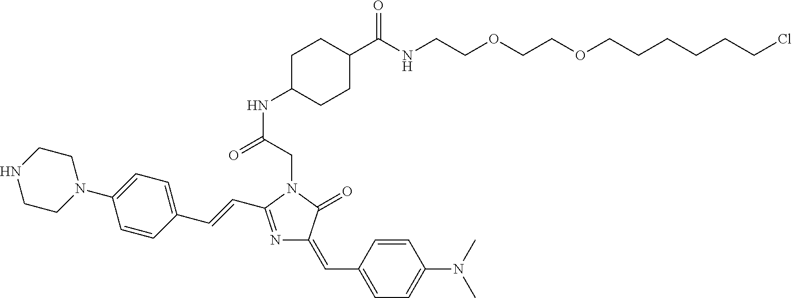 [SMILES: CN(C)c1ccc(/C=C2N=C(/C=C/c3ccc(N4CCNCC4)cc3)N(CC(=O)NC3CCC(C(=O)NCCOCCOCCCCCCCl)CC3)C/2=O)cc1] |
| 176 | 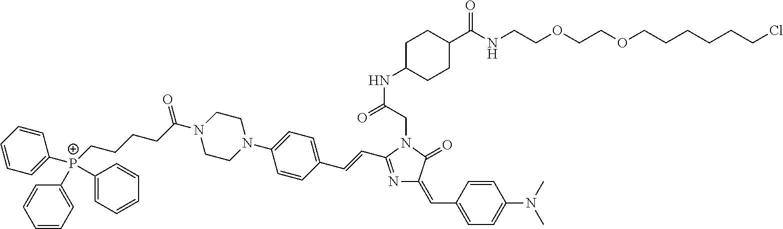 [SMILES: CN(C)c1ccc(/C=C2N=C(/C=C/c3ccc(N4CCN(C(=O)CCCC[P+](c5ccccc5)(c5ccccc5)c5ccccc5)CC4)cc3)N(CC(=O)NC3CCC(C(=O)NCCOCCOCCCCCCCl)CC3)C/2=O)cc1] |
| 177 | 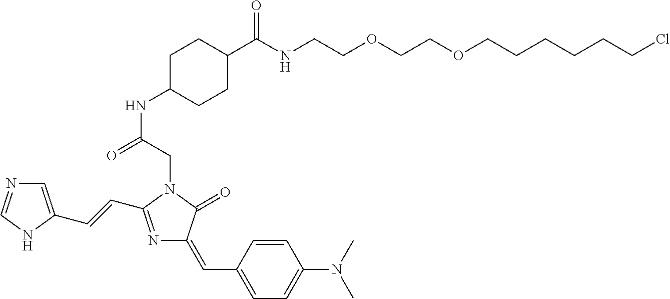 [SMILES: CN(C)c1ccc(/C=C2N=C(/C=C/c3cnc[nH]3)N(CC(=O)NC3CCC(C(=O)NCCOCCOCCCCCCCl)CC3)C/2=O)cc1] |
| 178 | 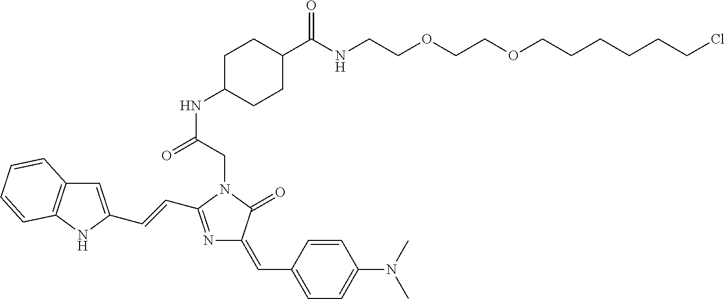 [SMILES: CN(C)c1ccc(/C=C2N=C(/C=C/c3cc4ccccc4[nH]3)N(CC(=O)NC3CCC(C(=O)NCCOCCOCCCCCCCl)CC3)C/2=O)cc1] |
| 179 | 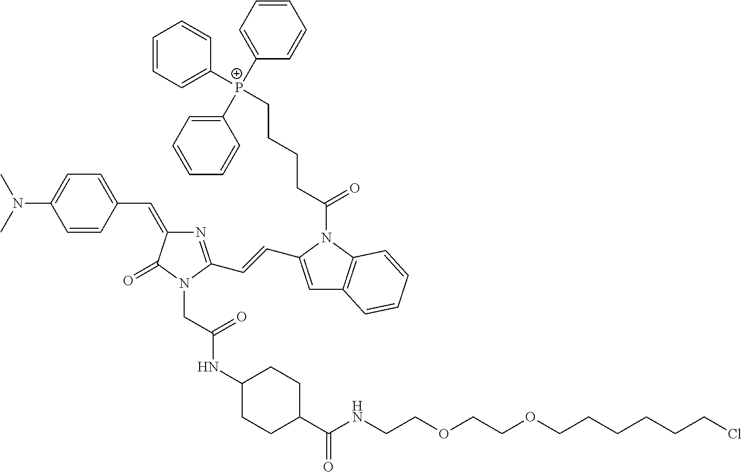 [SMILES: CN(C)c1ccc(/C=C2N=C(/C=C/c3cc4ccccc4n3C(=O)CCCC[P+](c3ccccc3)(c3ccccc3)c3ccccc3)N(CC(=O)NC3CCC(C(=O)NCCOCCOCCCCCCCl)CC3)C/2=O)cc1] |
| 180 | 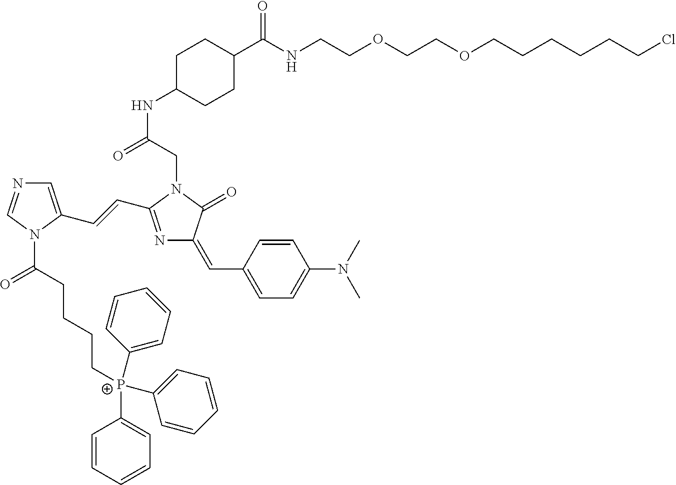 [SMILES: CN(C)c1ccc(/C=C2N=C(/C=C/c3cncn3C(=O)CCCC[P+](c3ccccc3)(c3ccccc3)c3ccccc3)N(CC(=O)NC3CCC(C(=O)NCCOCCOCCCCCCCl)CC3)C/2=O)cc1] |
| 181 | 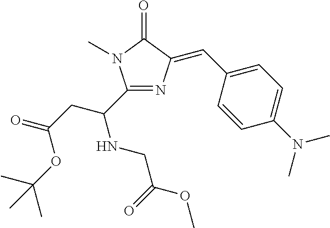 [SMILES: COC(=O)CNC(CC(=O)OC(C)(C)C)C1=N/C(=C\c2ccc(N(C)C)cc2)C(=O)N1C] |
| 182 | 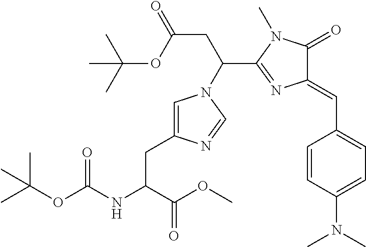 [SMILES: COC(=O)C(Cc1cn(C(CC(=O)OC(C)(C)C)C2=N/C(=C\c3ccc(N(C)C)cc3)C(=O)N2C)cn1)NC(=O)OC(C)(C)C] |
| 183 | 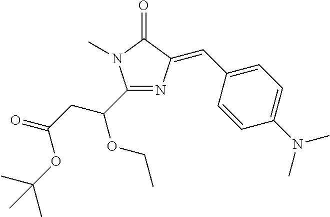 [SMILES: CCOC(CC(=O)OC(C)(C)C)C1=N/C(=C\c2ccc(N(C)C)cc2)C(=O)N1C] |
| 184 | 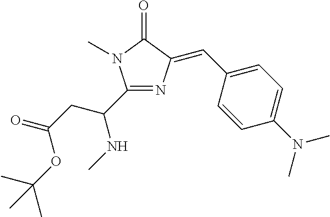 [SMILES: CNC(CC(=O)OC(C)(C)C)C1=N/C(=C\c2ccc(N(C)C)cc2)C(=O)N1C] |
| 185 | 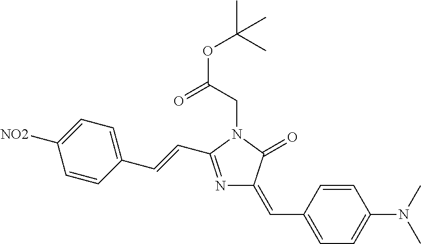 [SMILES: CN(C)c1ccc(/C=C2N=C(/C=C/c3ccc([N+](=O)[O-])cc3)N(CC(=O)OC(C)(C)C)C/2=O)cc1] |
| 186 | 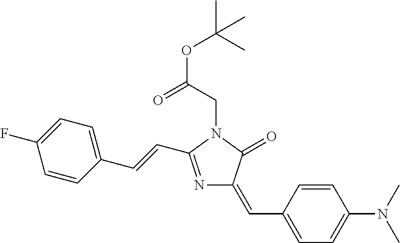 [SMILES: CN(C)c1ccc(/C=C2N=C(/C=C/c3ccc(F)cc3)N(CC(=O)OC(C)(C)C)C/2=O)cc1] |
| 187 | 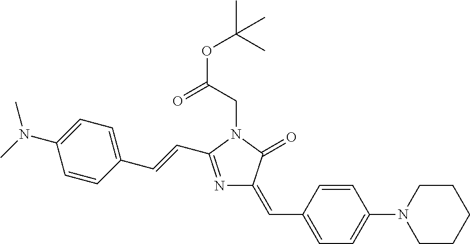 [SMILES: CN(C)c1ccc(/C=C/C2=NC(=C/c3ccc(N4CCCCC4)cc3)/C(=O)N2CC(=O)OC(C)(C)C)cc1] |
| 188 | 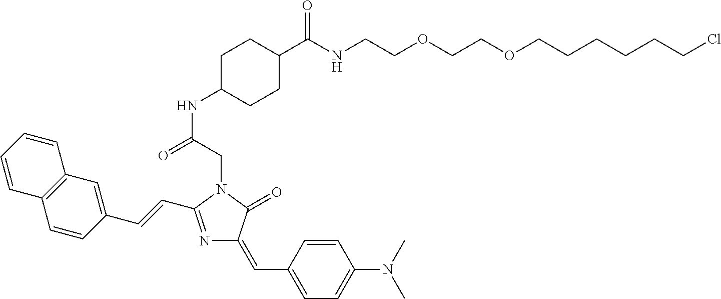 [SMILES: CN(C)c1ccc(/C=C2N=C(/C=C/c3ccc4ccccc4c3)N(CC(=O)NC3CCC(C(=O)NCCOCCOCCCCCCCl)CC3)C/2=O)cc1] |
| 189 | 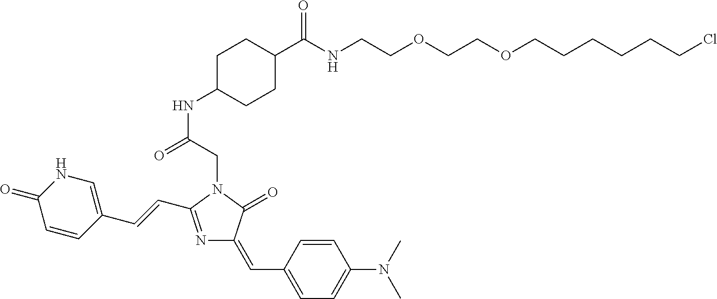 [SMILES: CN(C)c1ccc(/C=C2N=C(/C=C/c3ccc(=O)[nH]c3)N(CC(=O)NC3CCC(C(=O)NCCOCCOCCCCCCCl)CC3)C/2=O)cc1] |
| 190 | 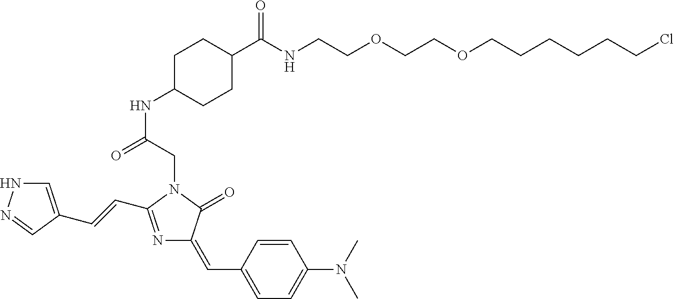 [SMILES: CN(C)c1ccc(/C=C2N=C(/C=C/c3cn[nH]c3)N(CC(=O)NC3CCC(C(=O)NCCOCCOCCCCCCCl)CC3)C/2=O)cc1] |
| 191 | 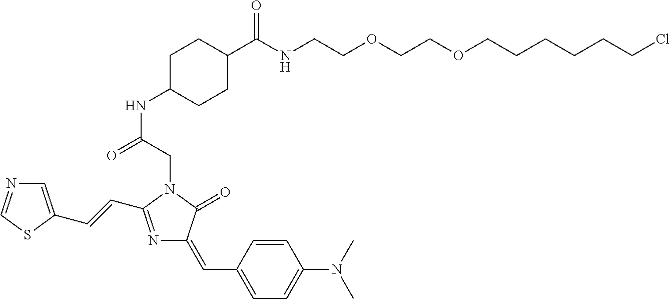 [SMILES: CN(C)c1ccc(/C=C2N=C(/C=C/c3cncs3)N(CC(=O)NC3CCC(C(=O)NCCOCCOCCCCCCCl)CC3)C/2=O)cc1] |
| 192 | 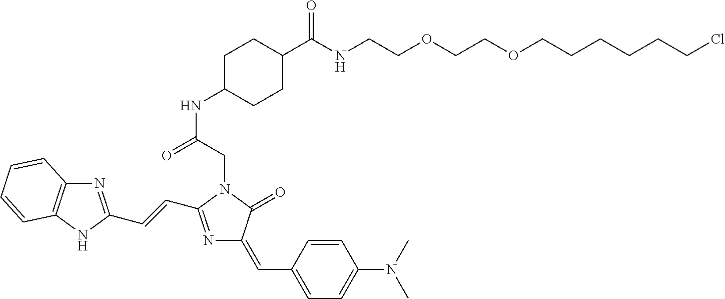 [SMILES: CN(C)c1ccc(/C=C2N=C(/C=C/c3nc4ccccc4[nH]3)N(CC(=O)NC3CCC(C(=O)NCCOCCOCCCCCCCl)CC3)C/2=O)cc1] |
| 193 | 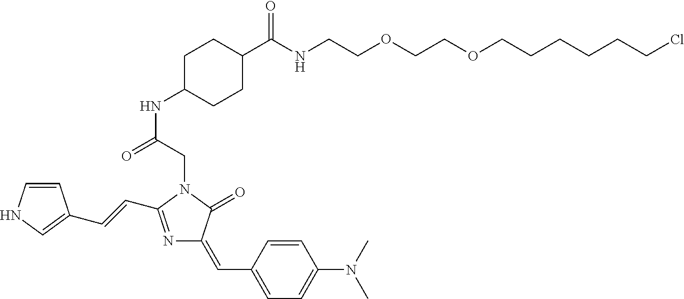 [SMILES: CN(C)c1ccc(/C=C2N=C(/C=C/c3cc[nH]c3)N(CC(=O)NC3CCC(C(=O)NCCOCCOCCCCCCCl)CC3)C/2=O)cc1] |
| 194 | 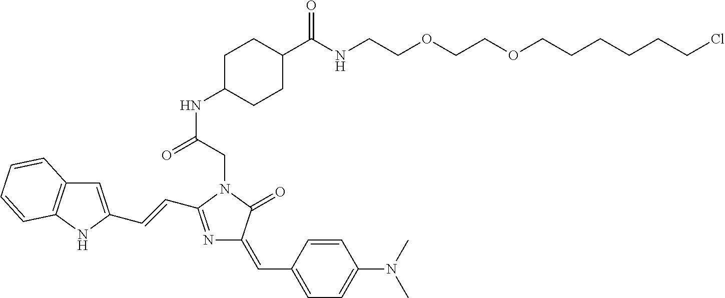 [SMILES: CN(C)c1ccc(/C=C2N=C(/C=C/c3cc4ccccc4[nH]3)N(CC(=O)NC3CCC(C(=O)NCCOCCOCCCCCCCl)CC3)C/2=O)cc1] |
| 195 | 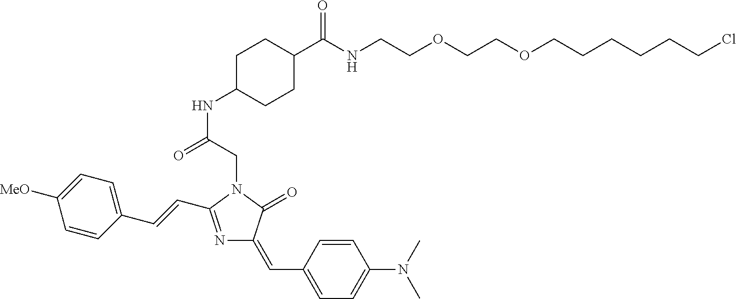 [SMILES: COc1ccc(/C=C/C2=NC(=C/c3ccc(N(C)C)cc3)/C(=O)N2CC(=O)NC2CCC(C(=O)NCCOCCOCCCCCCCl)CC2)cc1] |
| 196 | 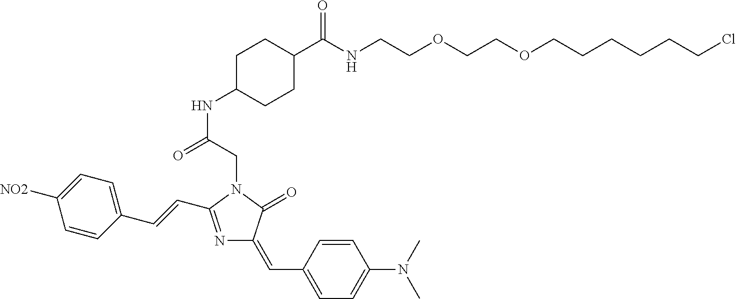 [SMILES: CN(C)c1ccc(/C=C2N=C(/C=C/c3ccc([N+](=O)[O-])cc3)N(CC(=O)NC3CCC(C(=O)NCCOCCOCCCCCCCl)CC3)C/2=O)cc1] |
| 197 | 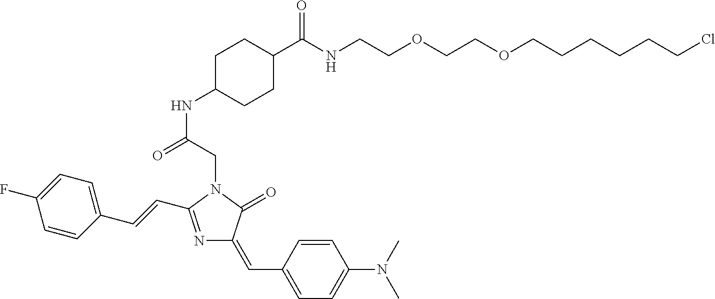 [SMILES: CN(C)c1ccc(/C=C2N=C(/C=C/c3ccc(F)cc3)N(CC(=O)NC3CCC(C(=O)NCCOCCOCCCCCCCl)CC3)C/2=O)cc1] |
| 198 | 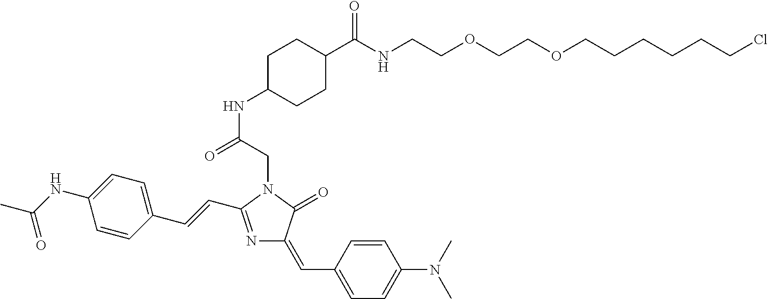 [SMILES: CC(=O)Nc1ccc(/C=C/C2=NC(=C/c3ccc(N(C)C)cc3)/C(=O)N2CC(=O)NC2CCC(C(=O)NCCOCCOCCCCCCCl)CC2)cc1] |
| 199 | 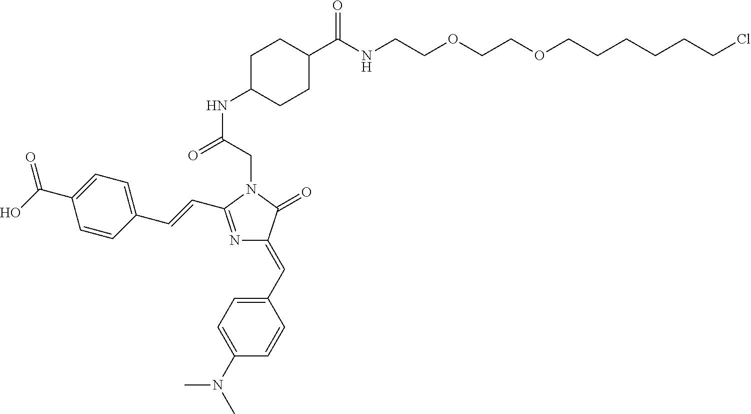 [SMILES: CN(C)c1ccc(/C=C2N=C(/C=C/c3ccc(C(=O)O)cc3)N(CC(=O)NC3CCC(C(=O)NCCOCCOCCCCCCCl)CC3)C\2=O)cc1] |
| 200 | 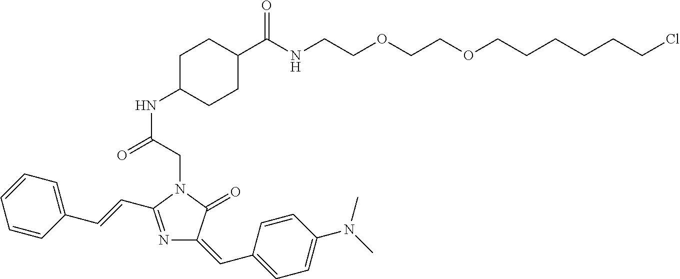 [SMILES: CN(C)c1ccc(/C=C2N=C(/C=C/c3ccccc3)N(CC(=O)NC3CCC(C(=O)NCCOCCOCCCCCCCl)CC3)C/2=O)cc1] |
| 201 | 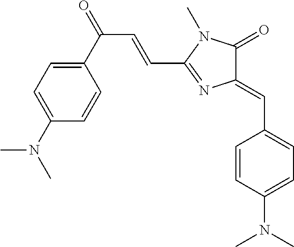 |
| 202 | 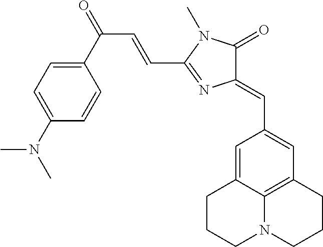 [SMILES: CN1C(=O)/C(=C/c2cc3c4c(c2)CCCN4CCC3)N=C1/C=C/C(=O)c1ccc(N(C)C)cc1] |
| 203 | 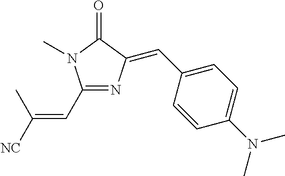 [SMILES: C/C(C#N)=C\C1=NC(=C\c2ccc(N(C)C)cc2)/C(=O)N1C] |
| 204 | 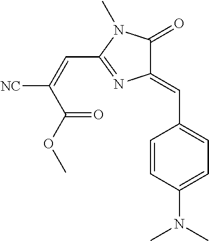 [SMILES: COC(=O)/C(C#N)=C\C1=NC(=C\c2ccc(N(C)C)cc2)/C(=O)N1C] |
| 205 | 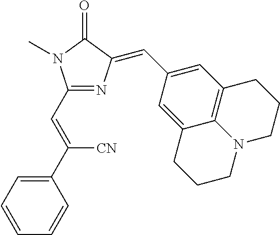 [SMILES: CN1C(=O)/C(=C/c2cc3c4c(c2)CCCN4CCC3)N=C1/C=C(\C#N)c1ccccc1] |
| 206 | 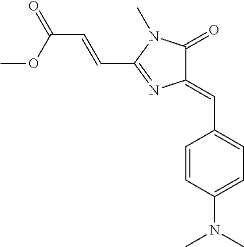 |
| 207 | 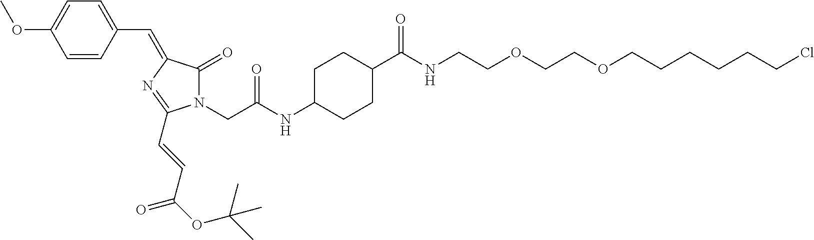 [SMILES: COc1ccc(/C=C2N=C(/C=C/C(=O)OC(C)(C)C)N(CC(=O)NC3CCC(C(=O)NCCOCCOCCCCCCCl)CC3)C\2=O)cc1] |
| 208 | 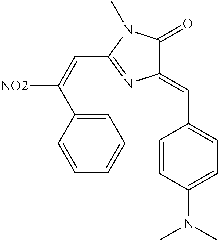 [SMILES: CN1C(=O)/C(=C/c2ccc(N(C)C)cc2)N=C1/C=C(\c1ccccc1)[N+](=O)[O-]] |
| 209 | 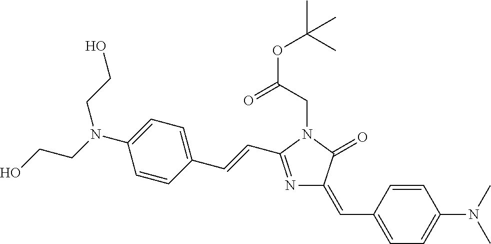 [SMILES: CN(C)c1ccc(/C=C2N=C(/C=C/c3ccc(N(CCO)CCO)cc3)N(CC(=O)OC(C)(C)C)C/2=O)cc1] |
| 210 | 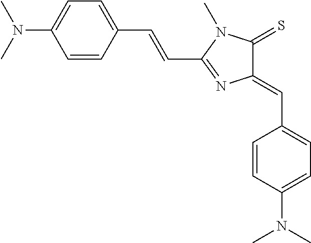 |
| 211 | 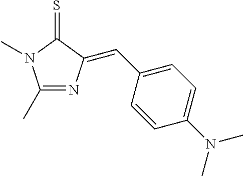 |
| 212 | 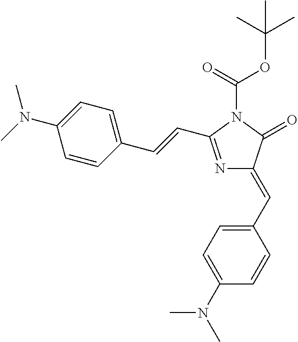 [SMILES: CN(C)c1ccc(/C=C2N=C(/C=C/c3ccc(N(C)C)cc3)N(C(=O)OC(C)(C)C)C\2=O)cc1] |
| 213 | 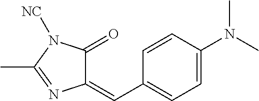 |
| 214 | 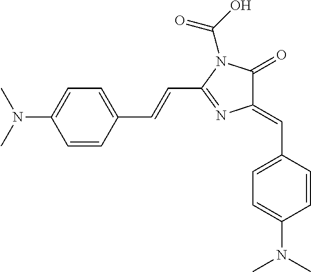 |
| 215 | 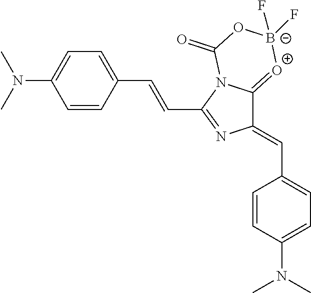 |
| 216 | 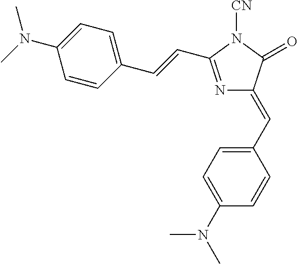 [SMILES: CN(C)c1ccc(/C=C2N=C(/C=C/c3ccc(N(C)C)cc3)N(C#N)C\2=O)cc1] |
| 217 | 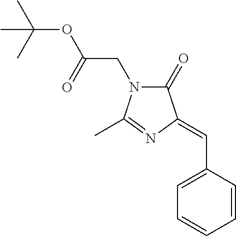 [SMILES: CC1=N/C(=C\c2ccccc2)C(=O)N1CC(=O)OC(C)(C)C] |
| 218 | 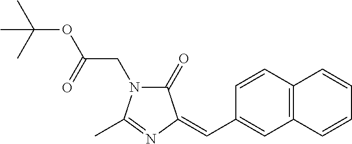 [SMILES: CC1=N/C(=C/c2ccc3ccccc3c2)C(=O)N1CC(=O)OC(C)(C)C] |
| 219 | 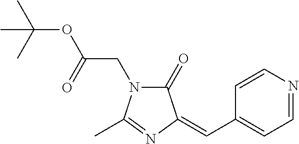 [SMILES: CC1=N/C(=C/c2ccncc2)C(=O)N1CC(=O)OC(C)(C)C] |
| 220 | 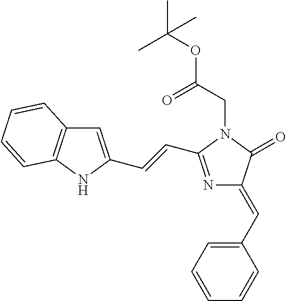 [SMILES: CC(C)(C)OC(=O)CN1C(=O)/C(=C/c2ccccc2)N=C1/C=C/c1cc2ccccc2[nH]1] |
| 221 | 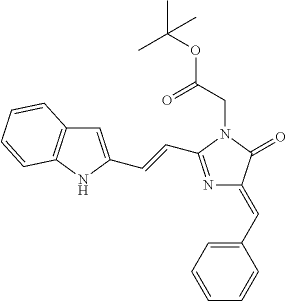 [SMILES: CC(C)(C)OC(=O)CN1C(=O)/C(=C/c2ccccc2)N=C1/C=C/c1cc2ccccc2[nH]1] |
| 222 | 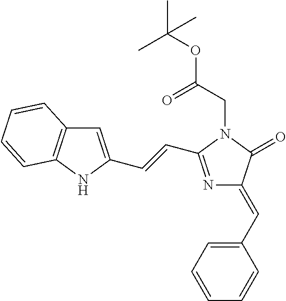 [SMILES: CC(C)(C)OC(=O)CN1C(=O)/C(=C/c2ccccc2)N=C1/C=C/c1cc2ccccc2[nH]1] |
| 223 | 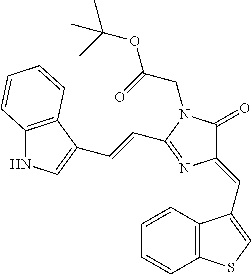 [SMILES: CC(C)(C)OC(=O)CN1C(=O)/C(=C/c2csc3ccccc23)N=C1/C=C/c1c[nH]c2ccccc12] |
| 224 | 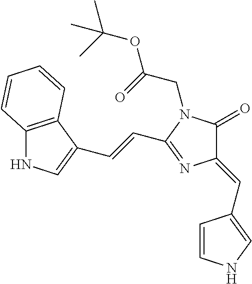 [SMILES: CC(C)(C)OC(=O)CN1C(=O)/C(=C/c2cc[nH]c2)N=C1/C=C/c1c[nH]c2ccccc12] |
| 225 | 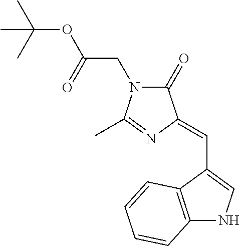 [SMILES: CC1=N/C(=C\c2c[nH]c3ccccc23)C(=O)N1CC(=O)OC(C)(C)C] |
| 226 | 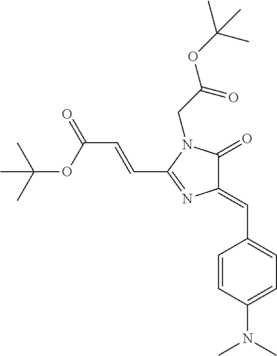 [SMILES: CN(C)c1ccc(/C=C2N=C(/C=C/C(=O)OC(C)(C)C)N(CC(=O)OC(C)(C)C)C\2=O)cc1] |
| 227 | 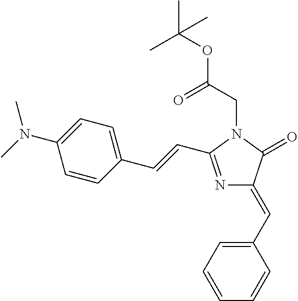 [SMILES: CN(C)c1ccc(/C=C/C2=NC(=C\c3ccccc3)/C(=O)N2CC(=O)OC(C)(C)C)cc1] |
| 228 | 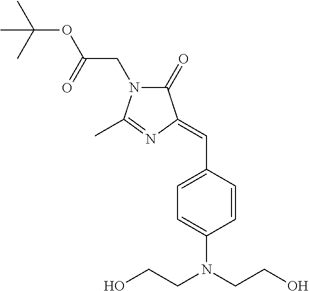 [SMILES: CC1=N/C(=C\c2ccc(N(CCO)CCO)cc2)C(=O)N1CC(=O)OC(C)(C)C] |
| 229 | 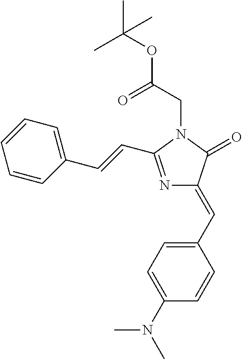 [SMILES: CN(C)c1ccc(/C=C2N=C(/C=C/c3ccccc3)N(CC(=O)OC(C)(C)C)C\2=O)cc1] |
| 230 | 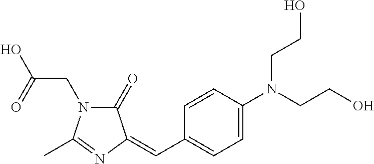 [SMILES: CC1=N/C(=C/c2ccc(N(CCO)CCO)cc2)C(=O)N1CC(=O)O] |
| 231 | 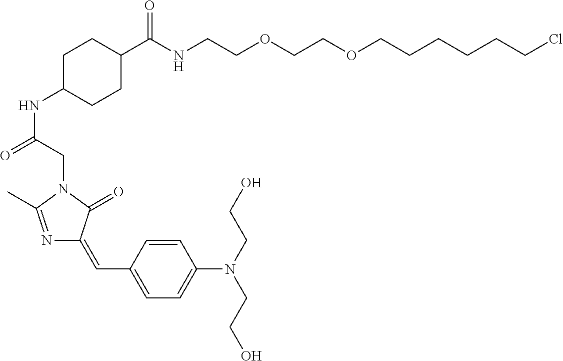 [SMILES: CC1=N/C(=C/c2ccc(N(CCO)CCO)cc2)C(=O)N1CC(=O)NC1CCC(C(=O)NCCOCCOCCCCCCCl)CC1] |
| 232 | 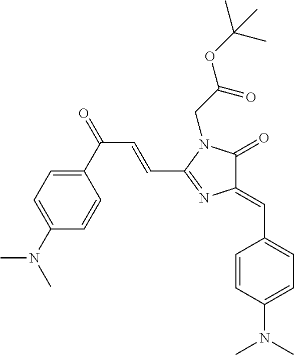 |
| 233 | 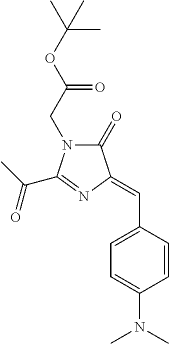 [SMILES: CC(=O)C1=N/C(=C\c2ccc(N(C)C)cc2)C(=O)N1CC(=O)OC(C)(C)C] |
| 234 | 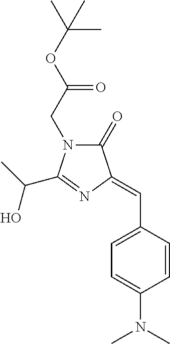 [SMILES: CC(O)C1=N/C(=C\c2ccc(N(C)C)cc2)C(=O)N1CC(=O)OC(C)(C)C] |
| 235 | 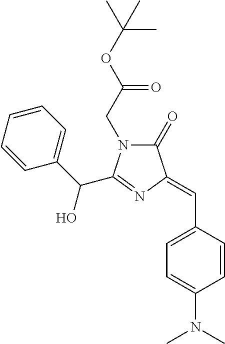 [SMILES: CN(C)c1ccc(/C=C2\N=C(C(O)c3ccccc3)N(CC(=O)OC(C)(C)C)C2=O)cc1] |
| 236 | 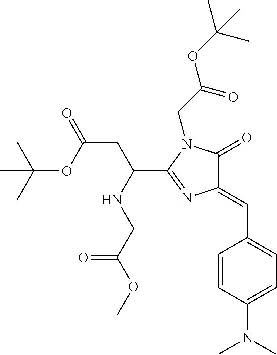 [SMILES: COC(=O)CNC(CC(=O)OC(C)(C)C)C1=N/C(=C\c2ccc(N(C)C)cc2)C(=O)N1CC(=O)OC(C)(C)C] |
| 237 | 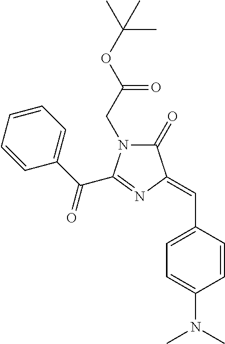 [SMILES: CN(C)c1ccc(/C=C2\N=C(C(=O)c3ccccc3)N(CC(=O)OC(C)(C)C)C2=O)cc1] |

In further embodiments as reported herein, protein aggregation and protein misfolding are detected by the disclosed compounds in test tubes. This detection can be conducted with or without covalent conjugation to proteins of interest. Normally, purified proteins are used in this detection. These proteins are subjected to in vitro conditions to induce protein misfolding and aggregation. Disclosed compounds can be added before, during, or after protein misfolding and aggregation. Fluorescence intensity can be recorded by fluorescence spectrophotometers or fluorescence microplate readers.

### Compounds 1-5:

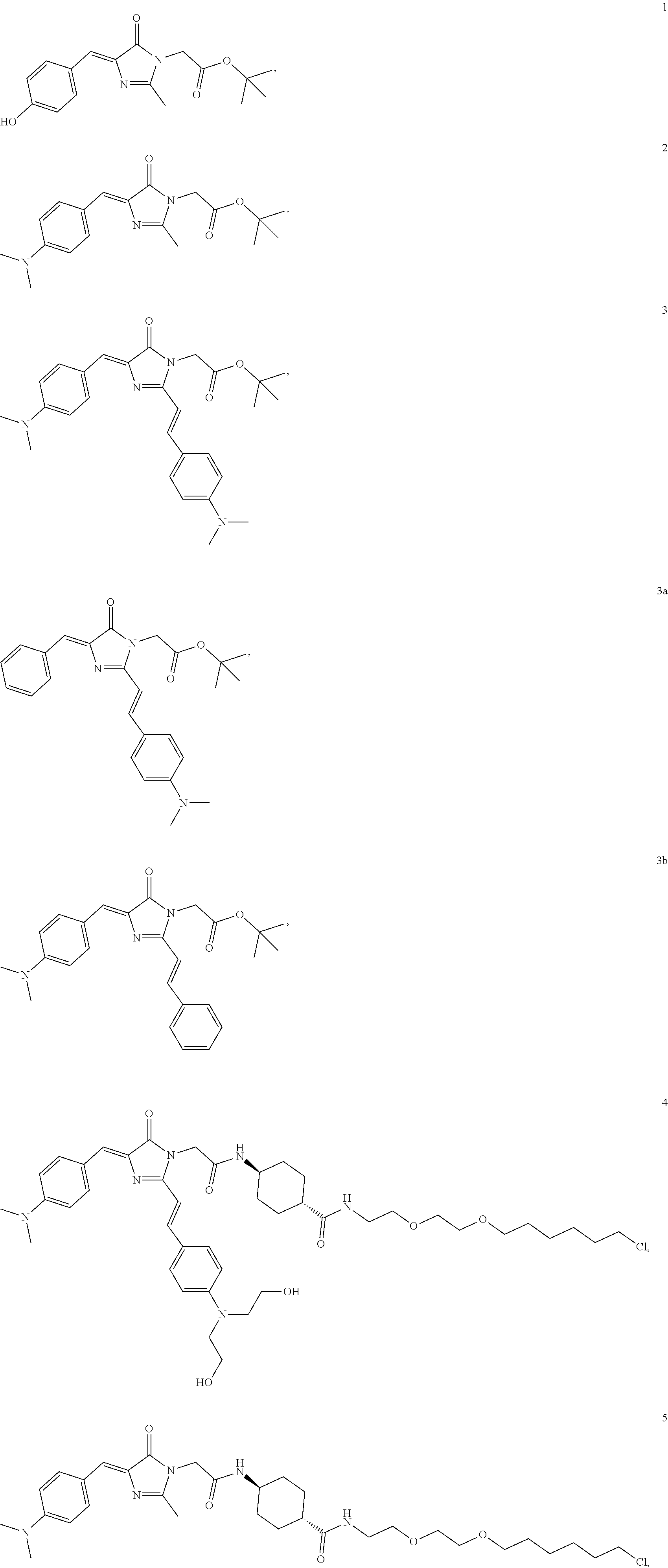

### Testing Examples

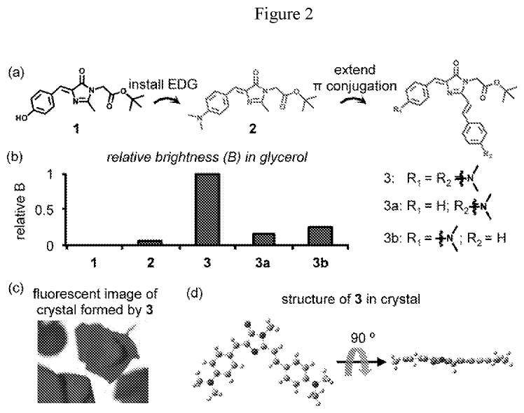

Compound 1, which contains HBI, requires both deprotonation to phenolate and restriction of bond rotations to emit fluorescence (FIG. 2a). Because the local pH inside misfolded and aggregated proteins is hardly basic enough to deprotonate phenol (pKa=8-10), the phenol in HBI may be substituted with an electron donating dimethylamino group, affording Compound 2 as reported above.

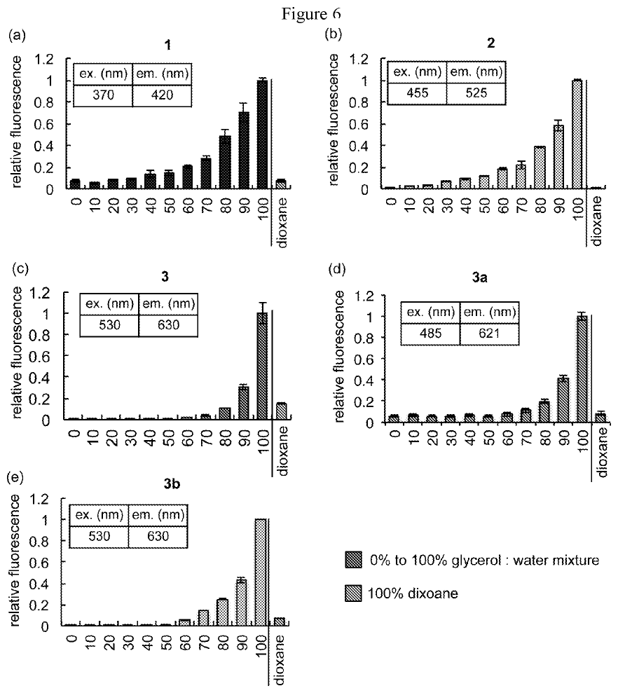

Both 1 and 2 exhibit elevated fluorescence in solvents containing increasing concentrations of glycerol (spectra shown in FIGS. 6a-b, 7a-b, 8a-b, and 9), which mimics the microenvironment in protein aggregates to inhibit TICT. However, the quantum yield (QY, Φ=0.001 for 1 and Φ=0.027 for 2 in glycerol, Table 2) was much lower than that of a GFP (Φ=0.79).

Further embodiments may extend n conjugation to both increase QY and restrict bond rotation (FIG. 2a). Such an embodiment is shown as Compound 3, reported above.

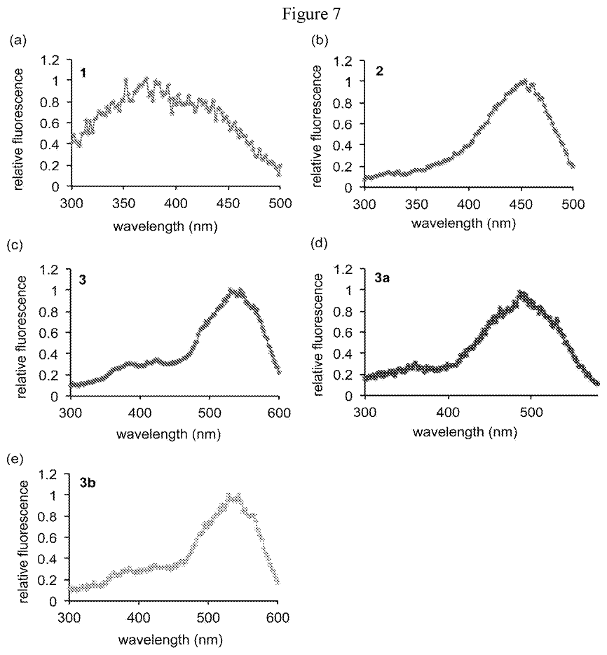

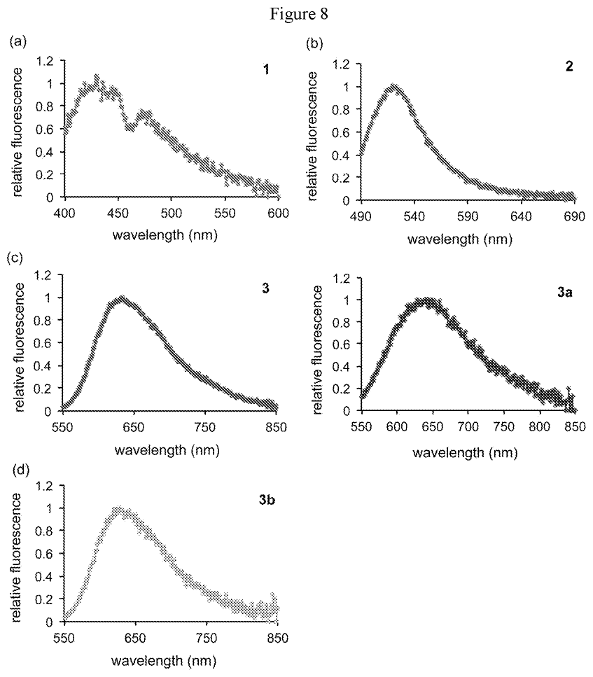

Compound 3 exhibited higher QY and brightness than 1 and 2 in glycerol (Φ=0.22, comparable to D of the Kaede protein as 0.33; brightness shown in FIG. 2b; spectra shown in FIGS. 6c, 7c, 8c, and 9; biophysical properties shown in Table 2).

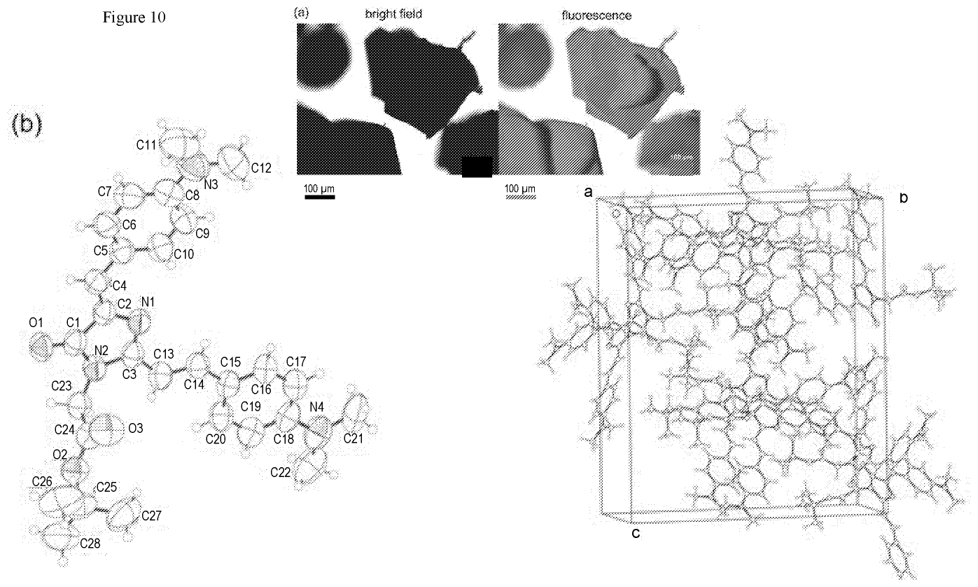

Crystals of 3 exhibited fluorescence. While not wishing to be bound by theory, this suggests its aggregation induced emission (AIE) feature (FIGS. 2c and 10a). The fluorescence may arise from the tight crystal packing that inhibits TICT of 3 (FIG. 10b).

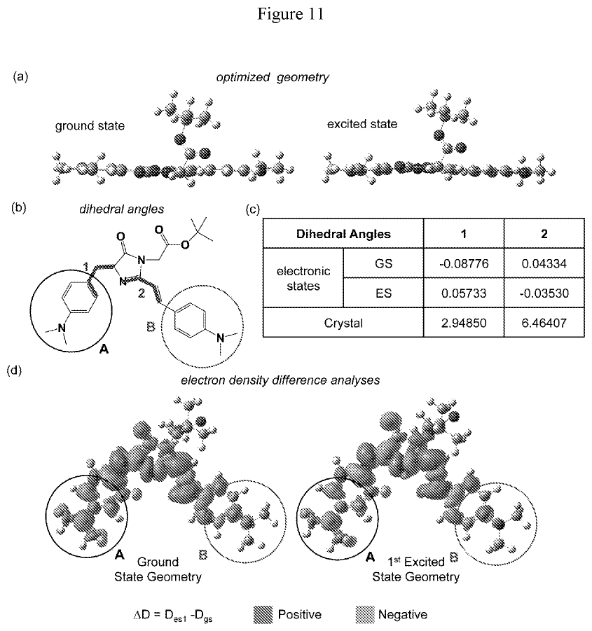

In the fluorescent crystal, 3 adopted a near planar structure (FIG. 2d). A time-dependent density functional theory calculation based on this structure (FIGS. 11a-c) showed that one dimethylamino benzyl played a major role in donating electrons at the S1 excited state (position A in FIG. 11d).

Compound 3a and Compound 3b were also prepared, as shown above.

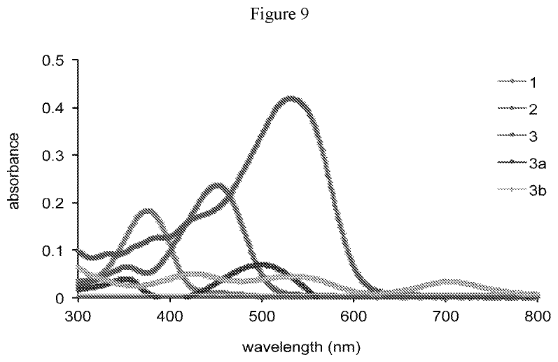

Compound 3a without electron-donating capacity at position A diminished quantum yield by ˜50%; whereas 3b without electron-donating capacity at position B does not affect quantum yield (Table 2). The absorptivity of both 3a and 3b was reduced (Table 2 and FIG. 9), yielding ˜5-fold decrease in its brightness (FIG. 2b and Table 2). Electron-donating groups in both A and B positions appeared useful for high quantum yield and brightness.

TABLE 2
The photophysical parameters of
fluorophores in the present invention.

| Com- | λₑₓ | λₑₘ | | λ_{abs} | | |
|---|---|---|---|---|---|---|
| pound | (nm) | (nm) | Φ | (nm)^{c} | ϵ(M⁻¹cm⁻¹)^{c} | B(M⁻¹cm⁻¹)^{d} |
| 1 | 370 | 429 | 0.001 | 376 | 20,592 | 20.6 |
| 2 | 450 | 521 | 0.027 | 452 | 23,718 | 640 |
| 3 | 530 | 629 | 0.221 | 530 | 39,049 | 8,786 |
| 3a | 485 | 621 | 0.1 | 500 | 13,863 | 1,386 |
| 3b | 530 | 630 | 0.232 | 533 | 9,004 | 2,089 |
| 4 | 530 | 628 | 0.230 | 534 | 36,241 | 8,734 |

aMaximum absorbance and extinction coefficient measured in glycerol.
bBrightness (B) was calculated using B = Φϵ.

TABLE 3
The geometry of 3 extracted from crystal structure. The total
energy at this geometry is −1531.67109379 Hartree.

| Atom | X (Å) | Y (Å) | Z (Å) |
|---|---|---|---|
| C | 8.589111 | −1.881432 | 1.224387 |
| H | 9.139859 | −1.768830 | 0.445329 |
| H | 8.998082 | −2.518740 | 1.813209 |
| H | 8.497810 | −1.039027 | 1.676078 |
| H | 7.677154 | −4.210787 | 1.409144 |
| C | 1.255841 | 2.555928 | −1.372002 |
| C | 2.079860 | 1.358102 | −1.047783 |
| C | 0.014408 | 0.720154 | −0.976268 |
| C | 3.430059 | 1.410384 | −0.977822 |
| H | 3.804300 | 2.224322 | −1.228115 |
| C | 4.388472 | 0.388601 | −0.571549 |
| C | 5.727878 | 0.741428 | −0.476613 |
| H | 5.985943 | 1.603805 | −0.707828 |
| C | 6.346334 | −1.447938 | 0.341371 |
| C | 4.994563 | −1.813307 | 0.214286 |
| H | 4.734045 | −2.680516 | 0.424980 |
| C | 4.042570 | −0.906792 | −0.220101 |
| H | 3.153915 | −1.172817 | −0.278479 |
| C | 6.889460 | −3.694201 | 1.224295 |
| H | 6.345074 | −3.645782 | 2.012474 |
| H | 6.392083 | −4.110123 | 0.517262 |
| C | −1.221120 | −0.053142 | −0.842227 |
| H | −2.028992 | 0.350472 | −1.064420 |
| C | −1.230528 | −1.321341 | −0.411810 |
| H | −0.405583 | −1.688712 | −0.190991 |
| C | −2.393740 | −2.195524 | −0.248500 |
| C | −2.222585 | −3.502827 | 0.192167 |
| H | −1.365663 | −3.791036 | 0.412277 |
| C | −3.271420 | −4.390282 | 0.317704 |
| H | −3.108393 | −5.258111 | 0.608236 |
| C | −4.572196 | −3.996638 | 0.008796 |
| C | −4.759688 | −2.683641 | −0.436285 |
| H | −5.616644 | −2.396456 | −0.654614 |
| C | −3.698918 | −1.799250 | −0.557339 |
| H | −3.857202 | −0.929174 | −0.848739 |
| H | −4.939809 | −6.694154 | −0.329524 |
| C | −1.183415 | 2.949098 | −1.368345 |
| H | −1.934115 | 2.471932 | −1.754659 |
| H | −0.988762 | 3.709282 | −1.938351 |
| C | −1.562630 | 3.441717 | 0.009391 |
| C | −3.264926 | 4.838528 | 1.141641 |
| C | −2.270442 | 5.770712 | 1.793963 |
| H | −1.500405 | 5.271937 | 2.076998 |
| H | −2.677748 | 6.188832 | 2.556554 |
| H | −2.003097 | 6.444013 | 1.164576 |
| C | −3.736996 | 3.730646 | 2.074251 |
| H | −4.412066 | 3.210115 | 1.635228 |
| H | −4.099555 | 4.118614 | 2.875078 |
| H | −2.995042 | 3.164813 | 2.301043 |
| C | −4.455907 | 5.578691 | 0.551176 |
| H | −4.145729 | 6.238137 | −0.073121 |
| H | −4.944598 | 6.008265 | 1.255781 |
| H | −5.027917 | 4.953304 | 0.100110 |
| N | 1.227433 | 0.271319 | −0.822233 |
| N | −0.043023 | 2.084575 | −1.330079 |
| N | 7.273613 | −2.362966 | 0.822083 |
| N | −5.636442 | −4.884588 | 0.135566 |
| O | 1.575820 | 3.732509 | −1.579503 |
| O | −2.646351 | 4.215190 | −0.068768 |
| O | −0.970195 | 3.171377 | 1.011101 |
| C | −6.986251 | −4.455920 | −0.216546 |
| H | −7.206796 | −3.659886 | 0.273176 |
| H | −7.609525 | −5.149602 | 0.002538 |
| H | −7.028281 | −4.273958 | −1.158477 |
| C | −5.403601 | −6.297338 | 0.409664 |
| H | −4.873618 | −6.386220 | 1.205418 |
| H | −6.246057 | −6.740703 | 0.535678 |
| C | 6.689652 | −0.155705 | −0.046086 |
| H | 7.579008 | 0.113535 | −0.016736 |

Synthesis of Example Compounds

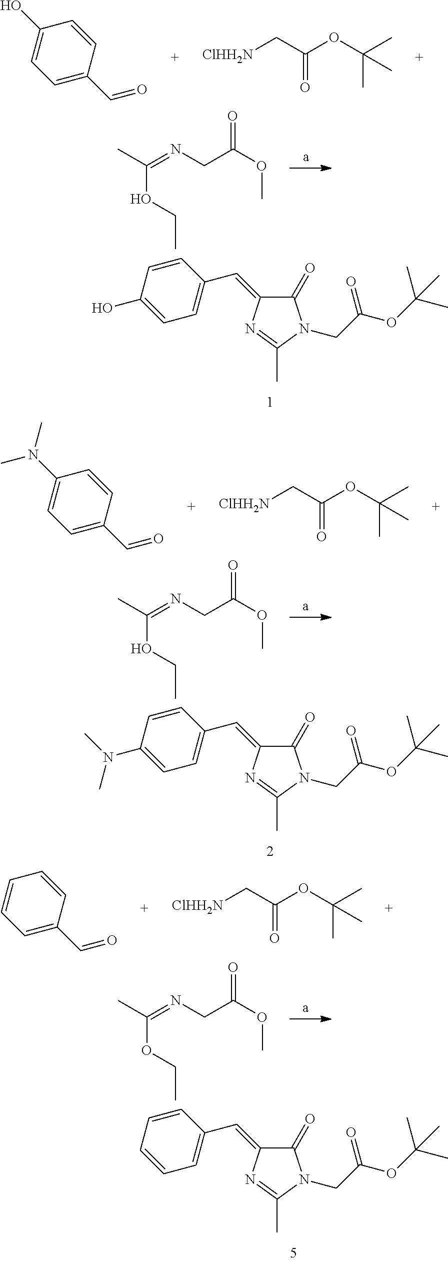

Scheme 1. Synthesis of GFP Mimic Precursors.

Cyclization reaction to form GFP core. Condition (a) glycine tert-butyl ester hydrochloride (1.1 eq) was combine with NaOH (1 eq) in EtOH and stirred for 1 hour at room temperature, aldehyde (1 eq) was added and stirred overnight, imidate (1 eq) was prepared and added in one portion.⁵ The reaction was stirred overnight, was then quenched by water and extracted with DCM. The organic fraction was collected and dried in vacuo. Compounds were further purified by flash chromatography (50% Ethyl Acetate, 50% Hexanes) to yield 1/2/5.

1: (Z)-tert-butyl 2-(4-(4-(hydroxy)benzylidene)-2-methyl-5-oxo-4,5-dihydro-1H-imidazol-1-yl)acetate. Crystalline orange solid. ¹H NMR (500 MHz, Chloroform-d) δ 8.11 (d, J=8.9, Hz, 2H), 6.95 (s, 1H), 6.85 (d, J=8.9 Hz, 2H), 4.40 (m, 3H), 2.27 (s, 3H), 1.44 (s, 9H). ¹³C NMR (126 MHz, Chloroform-d) δ 167.75, 161.59, 160.23, 135.95, 134.76, 126.91, 125.62, 116.27, 82.60, 42.32, 28.07, 15.53. [M+H]+: Calcd, 317.3570, Obsd, 317.1495

2: (Z)-tert-butyl 2-(4-(4-(dimethylamino)benzylidene)-2-methyl-5-oxo-4,5-dihydro-1H-imidazol-1-yl)acetate. Crystalline orange solid. ¹H NMR (500 MHz, Chloroform-d) δ 8.07 (d, J=8.9 Hz, 2H), 7.11 (s, 1H), 6.70 (d, J=8.8 Hz, 2H), 4.29 (s, 2H), 3.05 (s, 6H), 2.31 (s, 3H), 1.48 (s, 9H). ¹³C NMR (126 MHz, Chloroform-d) δ 170.11, 166.90, 158.03, 151.56, 134.27, 134.22, 129.41, 122.19, 111.72, 82.83, 42.15, 40.04, 27.99, 15.43. [M+H]+: Calcd, 344.1896, Obsd, 344.1983

5: (Z)-tert-butyl 2-(4-benzylidene-2-methyl-5-oxo-4,5-dihydro-1H-imidazol-1-yl)acetate. Crystalline yellow solid. H NMR (500 MHz, Chloroform-d) δ 8.05 (d, J=8.8, Hz, 2H), 7.30 (m, 3H), 7.01 (s, 1H), 4.19 (s, 2H), 2.23 (s, 3H), 1.39 (s, 9H). [M+H]+: Calcd, 301.3580, Obsd, 301.3367

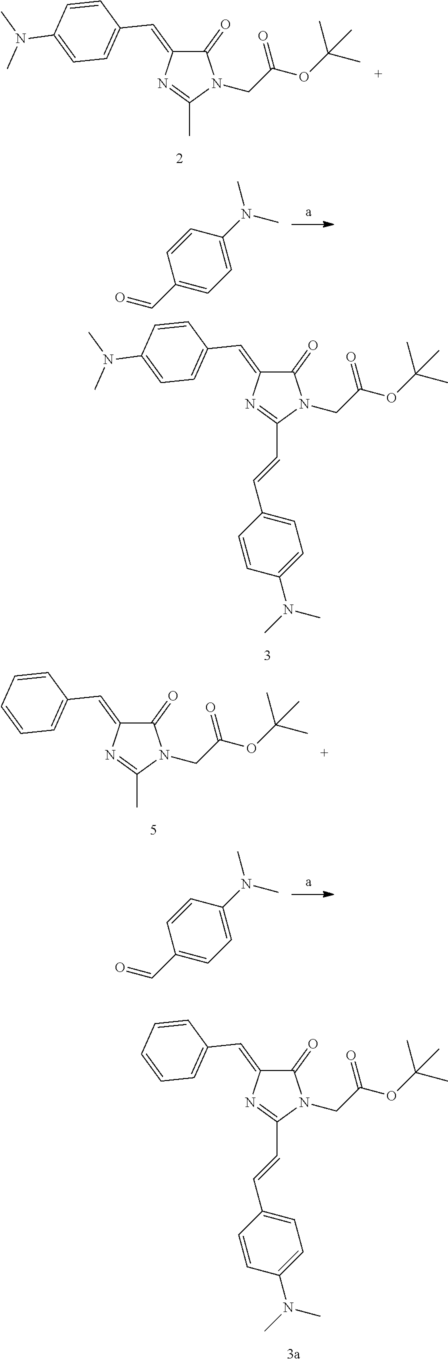

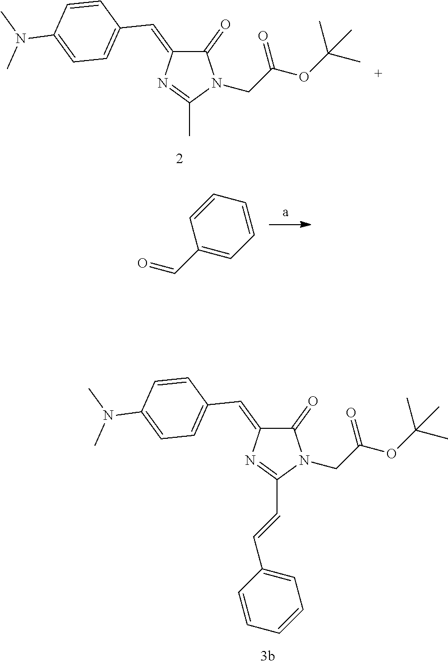

Scheme 2. General protocol for synthesis of in vitro GFP mimic probe. Carbon-carbon double bond formation via aldol condensation. Conditions, aldehyde (2eq), 2 or 5 (1 eq) were combine in dioxane under Argon. ZnCl₂ (0.01 eq) was added and the reaction was refluxed overnight. Solvent was removed and compounds were purified by flash chromatography (2:5, EA:Hexanes) to yield final GFP mimic probes 3/3a/3b.

3: tert-butyl 2-((Z)-4-(4-(dimethylamino)benzylidene)-2-((E)-4-(dimethylamino)styryl)-5-oxo-4,5-dihydro-1H-imidazol-1-yl)acetate. Red Solid. ¹H NMR (500 MHz, Chloroform-^{d}) δ 8.19 (d, J=8.6 Hz, 2H), 7.97 (d, J=15.6 Hz, 1H), 7.48 (d, J=8.7, 2H), 6.75 (d, J=8.6, 2H), 6.70 (d, J=8.6, 2H), 6.42 (d, J=15.6 Hz, 1H), 4.45 (s, 2H), 3.07 (d, J=15.1 Hz, 12H), 1.49 (m, 9H). 13C NMR (126 MHz, Chloroform-d) δ 170.36, 167.11, 156.60, 151.43, 151.34, 149.45, 139.93, 135.54, 134.83, 134.30, 132.48, 129.40, 129.17, 127.63, 123.53, 123.21, 112.91, 111.95, 111.85, 111.71, 107.58, 82.72, 60.39, 42.79, 42.37, 40.17, 40.11, 40.03, 31.24, 29.71, 28.03, 27.98, 21.06, 14.21. [M+H]+ Calcd, 475.2664, Obsd, 475.3517

3a: tert-butyl 2-((Z)-4-benzylidene-2-((E)-4-(dimethylamino)styryl)-5-oxo-4,5-dihydro-1H-imidazol-1-yl)acetate. Red oil. ¹H NMR (500 MHz, Chloroform-d) δ 8.25 (d, J=8.5, 2H), 8.15 (d, J=15.6 1H), 7.56-7.50 (m, 2H), 7.46 (t, J=7.6 Hz, 2H), 7.41-7.37 (m, 1H), 7.12 (s, 1H), 6.75 (d, J=8.6, 2H), 6.41 (d, J=15.5 Hz, LH), 4.46 (d, J=8.8 Hz, 2H), 3.07 (s, 6H), 1.50 (s, 9H). ¹C NMR (126 MHz, Chloroform-d) δ 171.14, 170.53, 166.78, 159.80, 151.79, 142.13, 139.38, 134.98, 132.96, 132.37, 132.31, 132.18, 129.88, 129.67, 129.62, 128.64, 128.56, 125.54, 123.02, 111.89, 110.99, 106.56, 82.98, 60.39, 42.34, 40.13, 34.67, 34.53, 31.65, 31.59, 29.71, 29.06, 28.00, 27.96, 25.28, 22.66, 21.10, 21.05, 20.70, 14.20, 14.12, 11.43. [M+H]+ Calcd, 432.2242, Obsd, 432.2269

3b: tert-butyl 2-((Z)-4-(4-(dimethylamino)benzylidene)-5-oxo-2-((E)-styryl)-4,5-dihydro-1H-imidazol-1-yl)acetate. Red solid. ¹H NMR (500 MHz, Chloroform-d) δ 8.20 (d, J=8.7 Hz, 2H), 8.01 (d, J=15.8 Hz, 1H), 7.65-7.57 (m, 2H), 7.42 (tt, J=8.5, 4.1 Hz, 3H), 7.20 (s, 1H), 6.80 (d, J=8.7, 2H), 6.68 (d, J=15.8 Hz, 1H), 4.47 (s, 2H), 3.10 (s, 6H), 1.48 (s, 911). ¹³C NMR (126 MHz, Chloroform-d) δ 170.11, 166.99, 155.57, 151.68, 139.12, 135.56, 135.04, 134.71, 129.72, 129.70, 128.94, 127.67, 122.82, 113.34, 111.84, 82.92, 67.10, 42.33, 40.10, 29.72, 27.98.

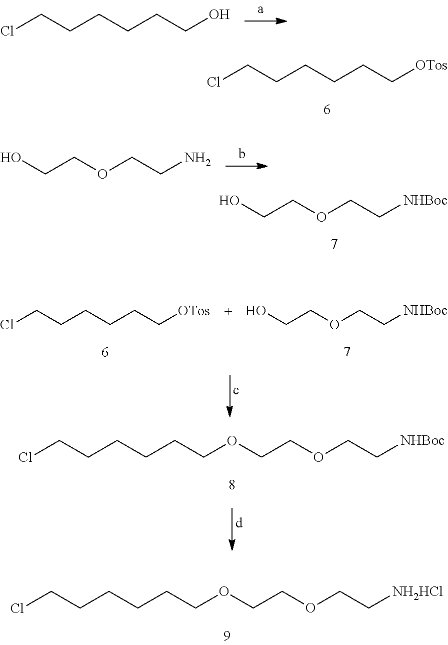

Scheme 3. Synthesis of PEG linker in large scale. Commercially available chloro-alcohol was converted into tosylate. Condition (a) 6-Chloro-1-hexanol (1.0 eq.), p-Toluenesulfonyl chloride (1.1 eq), 4-dimethylaminopyridine (0.1 eq) in pyridine for 1.5 hours at 0° C. The reaction mixture was extracted with diethyl ether against diluted HCl. The organic phase was collected and evaporated under reduced pressure, yielding crude colorless crystal 6. The product was carried on to the next step without further purification. Commercially available amino-alcohol was protected with t-butyl carbonate group. Condition (b) 2-(2-Aminoethoxy)ethanol (1.0 eq.), Di-tert-butyl dicarbonate (1.0 eq.) in methanol stirred for 3 h at room temperature. The reaction mixture was extracted with PBS Buffer against dichloromethane. The organic fraction was dried in vacuo. Compounds were further purified by flash chromatography (1:1 ethyl acetate/hexane) to yield 7, a colorless oil. 6 and 7 were combined to become protected PEG linker. Condition (c) 6 (1 eq), 7 (1.1 eq), and Potassium tert-butoxide (1 M in THF, 1.5 eq) in DMF stirred overnight at room temperature. The reaction was quenched with water and extracted with diethyl ether. The product was further purified by flash chromatography (1:2 ethyl acetate: hexane). Colorless oil 8 was obtained. Deprotection of 8 yielded the PEG amine hydrochloride. Condition (d) 8 (1 eq), HCl (4 M in Dioxane, 6 eq) stirred for 1 h at room temperature. Concentrated and then dried under high-vac to yield 9 a white solid.

6: 6-chlorohexyl 4-methylbenzenesulfonate. Colorless Crystal. (R₁=0.55, 1:9 EA:Hexane). ¹H NMR (500 MHz, CDCl₃) δ 7.73 (d, J=8.3 Hz, 2H), 7.31 (d, J=8.4 Hz, 2H), 3.97 (t, J=6.4 Hz, 2H), 3.43 (t, J=6.6 Hz, 2H), 2.39 (s, 3H), 1.73-1.54 (m, 4H), 1.39-1.24 (m, 4H). ¹³C NMR (126 MHz, CDCl₃) δ 144.73, 132.99, 129.81, 127.73, 70.37, 44.75, 32.19, 28.54, 26.05, 24.57, 21.51. [M+Na]+: Calcd, 313.0641, Obsd, 313.0639.

7: tert-butyl (2-(2-hydroxyethoxy)ethyl)carbamate. Colorless oil. ¹H NMR (500 MHz, CDCl₃) δ 5.29 (s, 1H), 3.73-3.64 (m, 2H), 3.56-3.45 (m, 4H), 3.31-3.15 (m, 3H), 1.45-1.34 (m, 9H). ¹C NMR (126 MHz, CDCl3) δ 156.26, 79.30, 72.32, 70.31, 61.55, 40.37, 28.42. HRMS for [M+Na]+: Calcd, 228.1212, Obsd, 228.1220.

8: tert-butyl (2-(2-((6-chlorohexyl)oxy)ethoxy)ethyl)carbamate. Colorless oil. ¹H NMR (500 MHz, CDCl₃) δ 5.02 (s, 1H), 3.58-3.47 (m, 8H), 3.42 (t, J=6.6 Hz, 2H), 3.30-3.24 (m, 2H), 1.78-1.70 (m, 2H), 1.60-1.53 (m, 2H), 1.46-1.38 (m, 11H), 1.38-1.30 (m, 2H). 13C NMR (126 MHz, CDCl₃) δ 156.03, 79.12, 71.28, 70.28, 70.22, 70.06, 45.02, 40.37, 32.56, 29.46, 28.44, 26.70, 25.44. [M+Na]+: Calcd, 346.1761, Obsd, 346.1765.

9: 2-(2-((6-chlorohexyl)oxy)ethoxy)ethanamine hydrochloride. White solid. ¹H NMR (500 MHz, DMSO) δ 8.26 (s, 3H), 3.65-3.58 (m, 4H), 3.56-3.52 (m, 2H), 3.51-3.46 (m, 2H), 3.36 (t, J=6.6 Hz, 2H), 2.94-2.87 (m, 2H), 1.73-1.65 (m, 2H), 1.51-1.44 (m, 2H), 1.41-1.33 (m, 2H), 1.33-1.25 (m, 2H). ¹C NMR (126 MHz, DMSO) δ 70.23, 69.70, 69.34, 66.58, 45.39, 38.37, 32.03, 29.06, 26.13, 24.93. [M+H]: Calcd, 224.1417, Obsd, 224.1411.

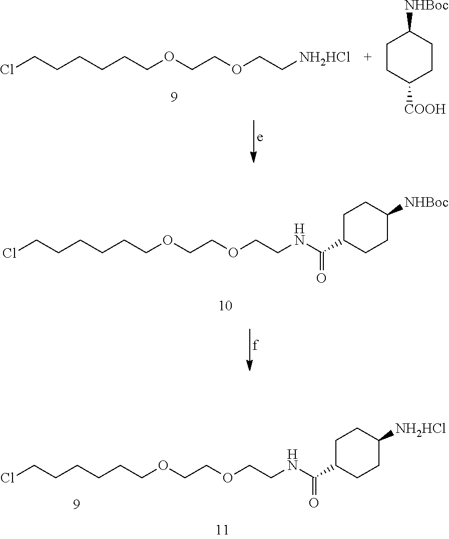

Scheme 4. Synthesis of rigid linker for small molecule probe. Commercially available cyclohexane linker was coupled with homemade PEG linker. Condition (e) 9 (1 eq.), trans-4-[[(1,1-Dimethylethoxy)carbonyl]amino]cyclohexanecarboxylic acid (1 eq.), dimethylaminopyridine (0.1 eq.), N-(3-Dimethylaminopropyl)-N′-ethylcarbodiimide hydrochloride (2 eq.) in triethylamine (4 eq.) stirred overnight at room temperature. The reaction mixture was quenched by water and extracted with DCM. Product was purified by flash chromatography (100% ethyl acetate). Sticky white solid 10 was obtained. Deprotection was of 10 yields final rigid linker amine hydrochloride. Condition (D 10 (1 eq.), in HCl (4 M in Dioxane, 6 eq.) for 1 h at room temperature. Reaction was concentrated and put under high-vac to yield fluffy white solid 11.

10: tert-butyl ((1r,4r)-4-((2-(2-((6-chlorohexyl)oxy)ethoxy)ethyl)carbamoyl)cyclohexyl)carbamate. Sticky white solid. ¹H NMR (500 MHz, CDCl₃) δ 5.99 (s, 1H), 4.45-4.38 (m, 1H), 3.61-3.49 (m, 8H), 3.47-3.37 (m, 5H), 2.10-2.03 (m, 2H), 2.01-1.95 (m, 1H), 1.93-1.85 (m, 2H), 1.79-1.71 (m, 2H), 1.62-1.51 (m, 4H), 1.47-1.40 (m, 11H), 1.39-1.32 (m, 2H), 1.13-1.03 (m, 2H). ¹C NMR (126 MHz, CDCl₃) δ 175.29, 155.23, 79.23, 71.32, 70.30, 70.07, 69.86, 49.13, 45.10, 44.63, 39.06, 32.80, 32.58, 29.54, 28.49, 26.75, 25.48. [M+H]⁺: Calcd, 449.2782, Obsd, 449.2783.

11: (1r,4r)-4-amino-N-(2-(2-((6-chlorohexyl)oxy)ethoxy)ethyl)cyclohexanecarboxamide hydrochloride. Fluffy white solid. ¹H NMR (500 MHz, DMSO) δ 8.19 (s, 3H), 7.87 (s, 1H), 3.61 (t, J=6.6 Hz, 2H), 3.52-3.43 (m, 4H), 3.40-3.33 (m, 4H), 3.20-3.13 (s, 2H), 2.96-2.87 (m, 1H), 2.11-2.03 (m, 1H), 2.01-1.94 (m, 2H), 1.78-1.65 (m, 4H), 1.53-1.43 (m, 2H), 1.43-1.25 (m, 8H). ¹C NMR (126 MHz, DMSO) δ 174.43, 70.18, 69.59, 69.45, 69.07, 48.75, 45.39, 42.46, 38.39, 32.03, 29.43, 29.07, 27.19, 26.12, 24.94. [M+H]⁺: Calcd, 349.2258, Obsd, 349.2257.

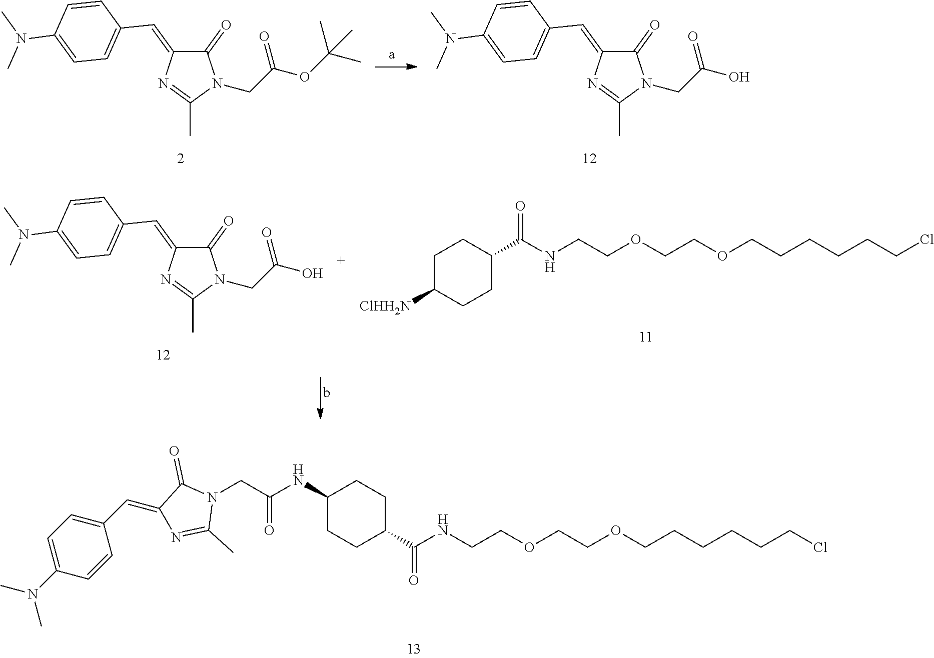

Scheme 5. Synthesis of GFP mimic precursors with rigid Halo linker. Deprotection of GFP core. Condition (a) 2 deprotected according to literature protocols to yield 12 which was used without further purification. GFP core was coupled with rigid Halo linker. Condition (b) 2 (1 eq), 11 (1.0 eq), dimethylaminopyridine (0.1 eq), N-(3-Dimethylaminopropyl)-N′-ethylcarbodiimide hydrochloride (2.0 eq), triethylamine (4.0 eq), overnight, RT. The reaction mixture was quenched by water and extracted with DCM. The organic fraction was collected and dried in vacuo. Compounds were further purified by flash chromatography (20:1, DCM:MeOH) to yield 13 a yellow powder.

13: (1r,4r)-N-(2-(2-((6-chlorohexyl)oxy)ethoxy)ethyl)-4-(2-((Z)-4-(4-(dimethylamino)benzylidene)-2-methyl-5-oxo-4,5-dihydro-1H-imidazol-1-yl)acetamido)cyclohexanecarboxamide. Yellow powder. ¹H NMR (500 MHz, Chloroform-d) δ 8.09 (d, J=8.8 Hz, 2H), 7.14 (s, 1H), 6.73 (d, J=8.9 Hz, 2H), 6.17-5.87 (m, 2H), 4.23 (s, 2H), 3.60-3.40 (m, 10H), 3.09 (s, 6H), 2.40 (s, 3H), 2.10-1.32 (m, 18H), 1.22-1.09 (m, 2H). ¹³C NMR (126 MHz, Chloroform-d) δ 174.99, 170.56, 166.29, 157.54, 151.88, 134.58, 133.55, 130.56, 121.83, 111.76, 71.26, 70.24, 69.99, 69.80, 48.20, 45.07, 44.76, 44.36, 40.05, 39.01, 32.51, 32.03, 29.45, 28.25, 26.68, 25.40, 15.59. [M+H]⁺: Calcd, 618.3344, Obsd, 618.3441.

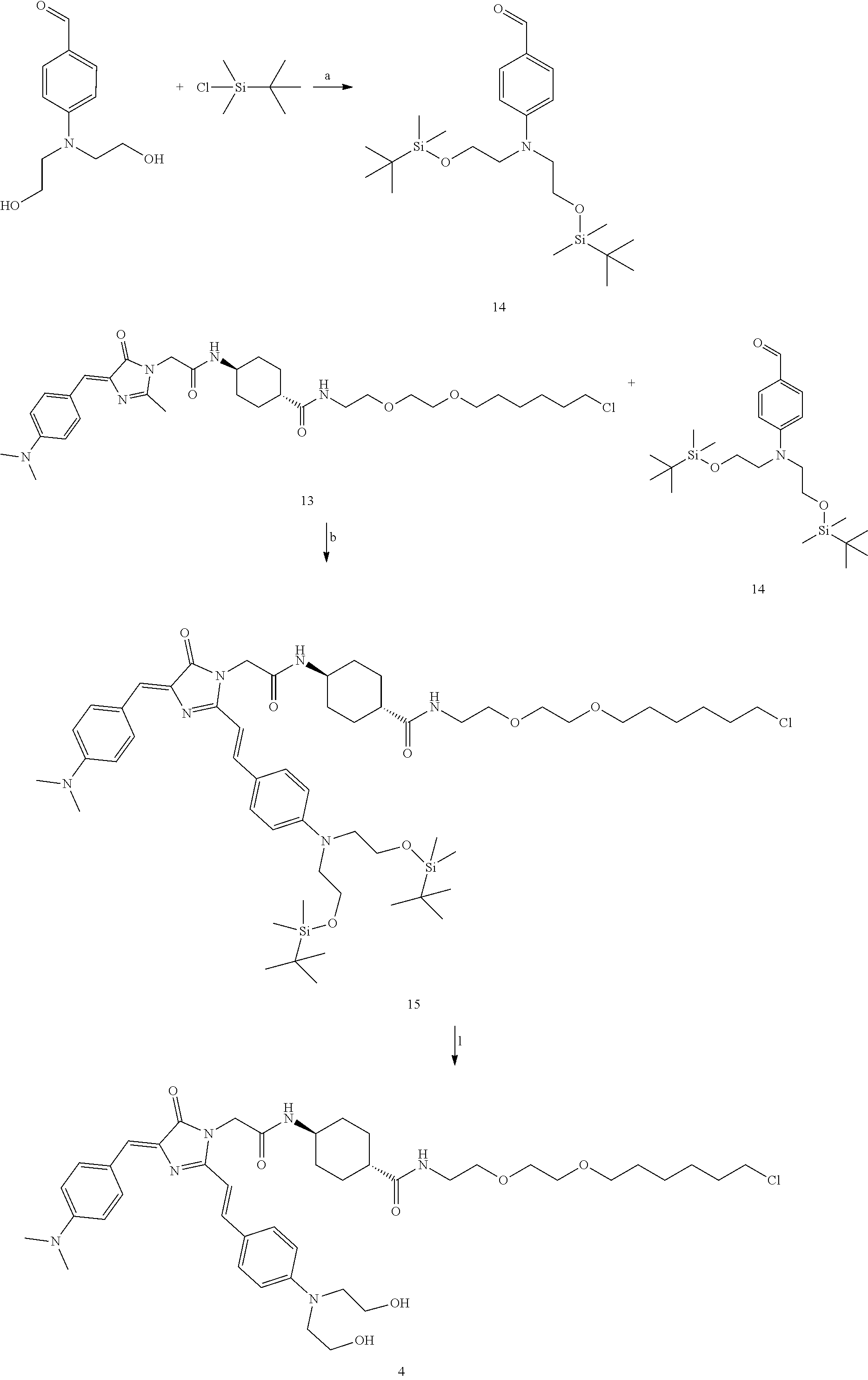

Scheme 6. Protocol for synthesis of in vivo GFP mimic probe. TBDMS protection. Conditions (a) 4-[Bis(2-hydroxyethyl)amino]benzaldehyde (1.0 eq) and imidazole (4.2 eq) were combined in DMF at room temperature, chloro(1,1-dimethylethyl)dimethylsilane (2.2 eq) was added and stirred for 1.5 hours. The reaction mixture was quenched with IM HCl and extracted with ether. The organic fraction was collected and dried in vacuo to yield 14 a transparent brown oil, product was used without further purification. Carbon-carbon double bond formation via aldol condensation. Conditions (b) 14 (2.0 eq), 13 (1.0 eq) were combine in in dioxane under Argon. ZnCl₂ (0.01 eq) was added and the reaction was refluxed overnight. Solvent was removed and compounds were purified by flash chromatography (20:1, DCM:MeOH) to yield 15. Product was immediately deprotected. Conditions (1) 15 (1.0 eq) was combined with TBAF (3.0 eq) in THF and stirred overnight. Compounds were purified by flash chromatography (10:1, DCM:MeOH) to yield 4 a pink solid.

4: (1r,4r)-4-(2-((Z)-2-((E)-4-(bis(2-hydroxyethyl)amino)styryl)-4-(4-(dimethylamino)benzylidene)-5-oxo-4,5-dihydro-1H-imidazol-1-yl)acetamido)-N-(2-(2-((6-chlorohexyl)oxy)ethoxy)ethyl)cyclohexanecarboxamide. Pink solid. ¹H NMR (500 MHz, DMSO-d6) δ 8.17 (d, J=8.3 Hz, 2H), 7.79 (d, J=15.5 Hz, 1H), 7.55 (d, J=8.7 Hz, 2H), 6.84 (s, 1H), 6.80 (dd, J=9.9, 2.7 Hz, 2H), 6.75 (dd, J=8.6, 5.5 Hz, 2H), 6.66 (d, J=15.5 Hz, 1H), 4.82 (s, 2H), 4.38 (s, 2H), 3.59-3.43 (m, 12H), 3.41-3.35 (m, 6H), 3.20-3.13 (m, 2H) 3.04 (s, 6H), 2.12-1.17 (m, 18H). ¹³C NMR (126 MHz, DMSO-d6) δ 175.24, 170.22, 166.39, 158.07, 151.49, 150.02, 139.33, 135.98, 134.26, 130.23, 125.20, 122.97, 122.84, 112.28, 111.89, 111.42, 108.29, 70.64, 70.04, 69.90, 69.55, 58.67, 58.60, 58.43, 53.59, 49.07, 48.09, 45.83, 43.53, 42.65, 38.85, 32.48, 32.07, 29.54, 28.61, 26.59, 25.40, 13.99. [M+H]+ Calcd, 809.4357, Obsd, 809.4369.

Examples of Application

Examples provided herein are indicative of, but should not be construed as limiting of, the overall invention.

Protein aggregation is a multistep process that includes aberrant conformations in the form of soluble oligomers, disordered or amorphous aggregates, and amyloid fibrils containing ordered hydrogen-bonded β-sheet structures. Embodiments provide methods for detecting proteins as misfolded oligomers, insoluble aggregates, and amyloid fibers. Principle of detection includes providing disclosed compounds (fluorescent protein chromophores) that fluoresce when placed in an environment of enhanced rigidity indicative of protein aggregation in a local microenvironment. In a typical embodiment fluorescence occurs when the rigid microenvironment causes inhibition of non-radiative decay via twisted-intramolecular charge transfer (TICT). This principle can be applied to detection conducted in test tubes and live cells.

Detection as reported herein may be conducted using purified proteins in test tubes or in live cells bearing proteins of interest.

In one embodiment, protein aggregation and protein misfolding are detected by disclosed compounds in test tubes. This detection can be conducted with or without covalent conjugation to proteins of interest. Normally, purified proteins are used in this detection. These proteins are subjected to in vitro conditions to induce protein misfolding and aggregation. Disclosed compounds can be added before, during, or after protein misfolding and aggregation. Fluorescence intensity can be recorded by fluorescence spectrophotometers or fluorescence microplate readers.

In one embodiment, aggregation of proteins of interest is detected in live cells either transiently or stably expressing these proteins. The inventors disclose the compounds with the following methods (varying R₃ groups) to conjugate with proteins of interest that are of at least one of the aberrant conformations in the form of soluble oligomers, disordered or amorphous aggregates, and amyloid fibrils containing ordered hydrogen-bonded β-sheet structures. These compounds can covalently conjugate with the Halo-Tag fusion domain (a product from Promega Inc) via a chloroalkane linker, the SNAP-Tag fusion domain (a product from NEB Inc.) via an O6-benzylguanine linker, the CLIP-Tag fusion domain (a product from NEB Inc.) via an O6-benzylcytosine linker, exposed cysteine via maleimide, or exposed lysine via N-Hydroxysuccinimide (Succinimidyl) esters. Fluorescence detection can be conducted by fluorescence spectrophotometers, fluorescence microplate readers, and epifluorescence or confocal fluorescence microscopes.

In one embodiment, aggregation of cellular proteins is detected in live cells. The inventors disclose the compounds with the following methods (varying R₃ groups) to bind to cellular proteins that are of at least one of the aberrant conformations in the form of soluble oligomers, disordered or amorphous aggregates, and amyloid fibrils containing ordered hydrogen-bonded β-sheet structures. These compounds can non-covalently bind to these conformations using a thioflavin-T guiding group or a tert-Butyloxycarbonyl guiding group. Fluorescence detection can be conducted by fluorescence spectrophotometers, fluorescence microplate readers, and epifluorescence or confocal fluorescence microscopes.

Applications as reported above can also be conducted in fixed cell samples with or without permeabilized cellular membranes.

Embodiments further provide a kit for detecting at least one of the following aberrant protein conformations: misfolded oligomers, amorphous insoluble protein aggregates, amyloid fibrils containing ordered hydrogen-bonded β-sheet structures. The kit includes the following components: one or more of disclosed fluorescent protein chromophores as reported herein of a known concentration in stock solutions of Dimethyl Sulfoxide or Ethanol, one or more standard protein samples that form one of the abovementioned aberrant protein conformations, and optionally, instructions for use in detecting at least one of one of the abovementioned aberrant protein conformations.

Compounds Detecting Misfolded Oligomers in Test Tubes (AggGlow Method).

The inventors have designed and synthesized an HBI analogue, 3, which harbors an extended π conjugation to both increase QY and restrict bond rotation (FIG. 2).^{27,35,36,40,41} 3 exhibits quantum yield (ϕ) value of 0.22 in glycerol, a viscous solvent that mimics misfolded oligomers, comparable to D of the Kaede protein as 0.33.4 The molar extinction coefficient (ε) of 3 is 39,049 M⁻¹·cm⁻¹, about half of mCherry (72,000 M⁻¹·cm⁻¹). The inventors also determined photostability of 3 by measuring its absorbance spectra in glycerol and protein aggregates. When irradiated using 5 mW laser beam of 543 nm, absorbance of 3 at 530 un only exhibits a 10% drop, suggesting that 3 is a photostable fluorophore. These photochemical properties make 3 suited for fluorescence microscopy.

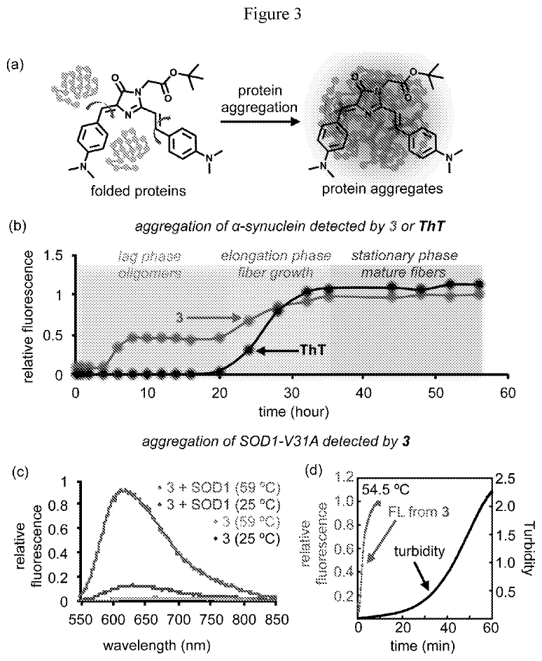

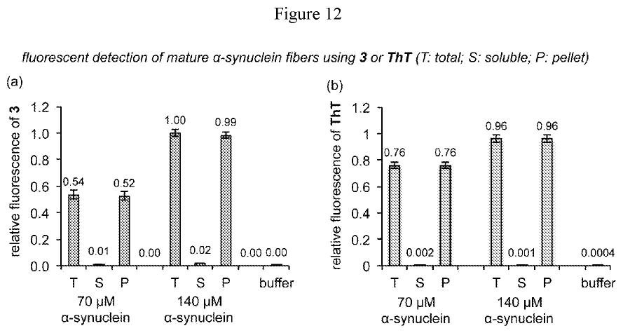

Determination of Compound 3's detection of protein aggregation via fluorescence increase (FIG. 3a), is demonstrated by analysis with the model protein α-synuclein (α-syn), whose aggregation is associated with Parkinson's disease. In the presence of 3, aggregation of α-syn into mature fibers induced fluorogenic signal (FIG. 12a). Fluorescence intensity of 3 was stoichiometric (1.0 for 140 μM α-syn and 0.52 for 70 μM α-syn in FIG. 12a); whereas such stoichiometry was absent using Thioflavin T (ThT), a widely used fluorogenic probe to detect amyloid fibers (FIG. 12b).

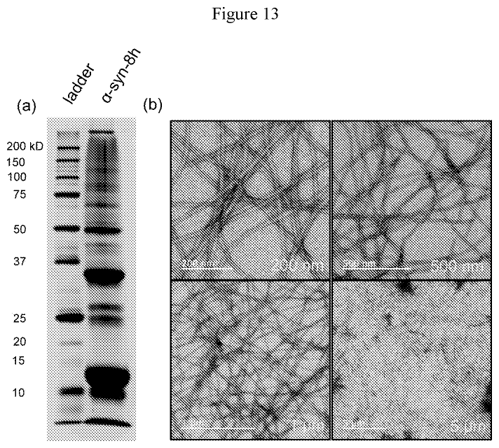

Compound 3 was also used to monitor kinetics of α-syn aggregation. Purified α-syn typically aggregates via a three-step process: formation of soluble oligomers, growth of amyloid fibers, and maturation of fibers (FIG. 3b). Although ThT detects growth and maturation of fibers, it fails to detect soluble oligomers that are increasingly speculated to be the toxic species in PD (black curve in FIG. 3b). By contrast, fluorescence of 3 started to increase at 4 h and reached a plateau at 8 h (red curve in FIG. 3b). At this time point, formation of soluble oligomers was evidenced by a photo-induced crosslinking experiment (FIG. 13a). Furthermore, a second phase of fluorescence increase was observed at 20 h and leveled off at 36 h (red curve in FIG. 3b). These signal changes coincided with the time points of fiber growth and maturation as observed by ThT (black curve of FIG. 3b). Thus, these data strongly indicate that 3 could detect multiple stages of protein aggregation.

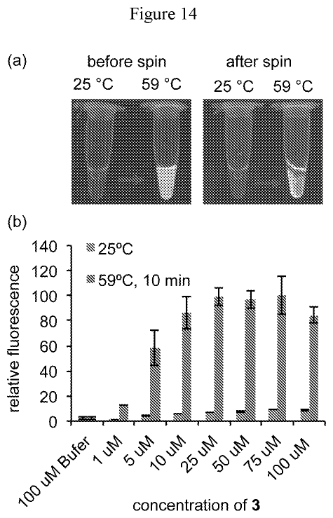

In addition to α-synuclein, Compound 3 may detect aggregates formed by globular proteins, by using mutant superoxide dismutase 1 (SOD1), whose aggregation is commonly found in ALS disease. The recently discovered SOD1(V31A) mutant may be used to confirm this. After 20 min incubation at 59° C., an 8 to 10-fold fluorescence intensity increase (FIGS. 3c and 14a) was observed using a wide concentration range of 3 (FIG. 14b). The fluorescence signal was primarily found in the fraction of insoluble protein aggregates (FIG. 14a), confirming the physical association between 3 and SOD1 aggregates. In addition, the kinetics of fluorescence increase from 3 was faster than that of turbidity whose signal originates from insoluble protein aggregates (FIG. 3d). This suggests that 3 can detect both misfolded soluble proteins and insoluble aggregates.

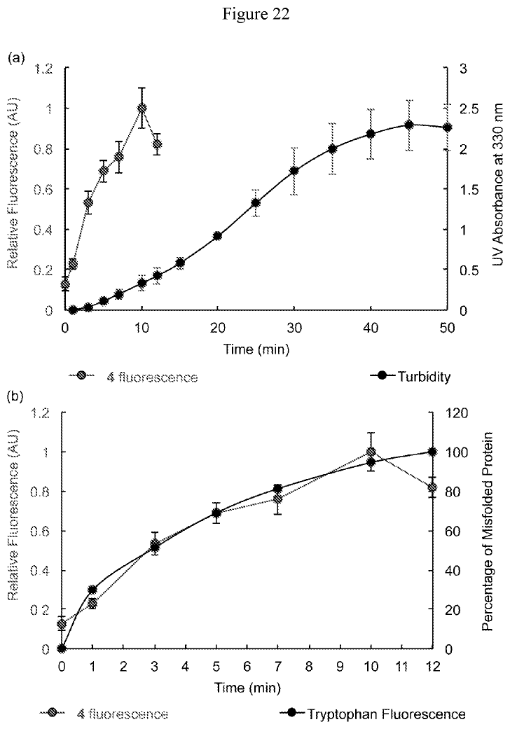

3 detects soluble oligomers via fluorescence increase. To test this point, the inventors chose α-synuclein (α-syn), whose aggregation is associated with Parkinson's disease.⁴² Purified α-syn aggregates via a three-step process: formation of soluble oligomers, growth of amyloid fibers, and maturation of fibers (FIG. 3b). Although Thioflavin-T (ThT) monitors growth and maturation of fibers,⁴³ it fails to detect soluble oligomers (black curve in FIG. 3b). By contrast, fluorescence of 3 started to increase at 4 h (gray curve in FIG. 3b), at which soluble oligomers was detected by a chemical crosslinking assay (FIG. 13). Furthermore, a second phase of fluorescence increase was observed at 20 h and leveled off at 36 h (gray curve in FIG. 3b). These signal changes coincided with the time points of fiber growth and maturation as observed by ThT (black curve of FIG. 3b). The inventors also demonstrated that 3 could detect misfolded oligomers formed by globular proteins, by using mutant superoxide dismutase 1 (SOD1), whose aggregation is commonly found in ALS disease.⁴⁴ Using the recently discovered V31A mutant,⁴⁵ the inventors observed 8 to 10-fold fluorescence intensity increase (5 from 0.02 to 0.22, FIG. 3c) when purified SOD1-V31A was incubated at 59° C. Further, the inventors found the kinetics of fluorescence increase from 3 was faster than that of turbidity whose signal originates from insoluble protein aggregates (FIGS. 3d and 22a) and identical to that of protein misfolding as measured by tryptophan fluorescence (FIG. 22b). These data collectively suggest that 3 could detect misfolded oligomers.

Compounds Detecting Misfolded Oligomers in Live Cells (AggGlow Method).

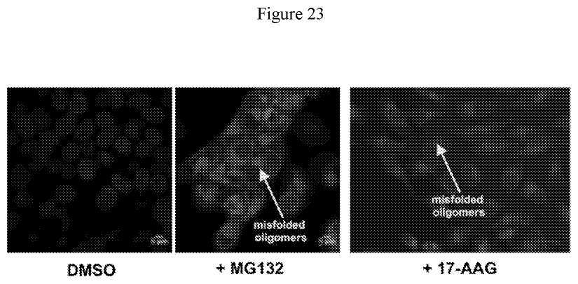

3 is used to monitor stress-induced proteome aggregation in HEK293T (human embryonic kidney) cells with a proteasome inhibitor MG132. Inhibition of proteasome has been shown to form cytosolic misfolded oligomers and insoluble aggregates of cellular proteins. When 5 μM of MG132 was used to treat HEK293T cells in the presence of 2.5 μM 3, obvious fluorescence pattern was developed in the cytosol of cells as a result of proteasome inhibition (FIG. 23). The diffusive fluorescence structure indicates misfolded oligomers and the granular fluorescence structure indicates insoluble aggregates. Similar to this application, 3 was used to monitor stress-induced proteome aggregation in HeLa cells with a heat-shock protein 90 (Hsp90) inhibitor 17-AAG. Inhibition of Hsp90 has been shown to form cytosolic misfolded oligomers and insoluble aggregates of cellular proteins. When 1 μM of 17-AAG was used to treat HeLa cells in the presence of 2.5 μM 3, obvious fluorescence pattern was developed in the cytosol of cells as a result of Hsp90 inhibition (FIG. 23).

Compounds Detecting Misfolded Oligomers of Specific Proteins in Live Cells (AggTag method).

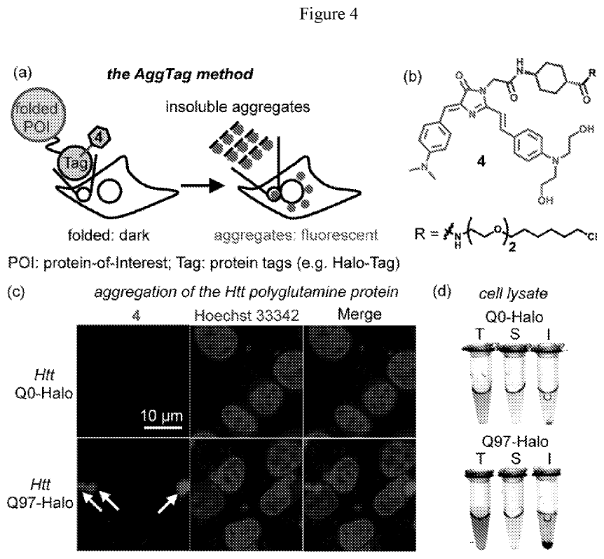

The inventors further examined using a turn-on fluorescence to monitor aggregation of a protein-of-interest (POI) using in live cells. For this example, the inventors genetically fused Halo-Tag to the POI and synthesized 4 for bioorthogonal conjugation (FIG. 4a, hereafter referred to as AggTag method). Compound 4:

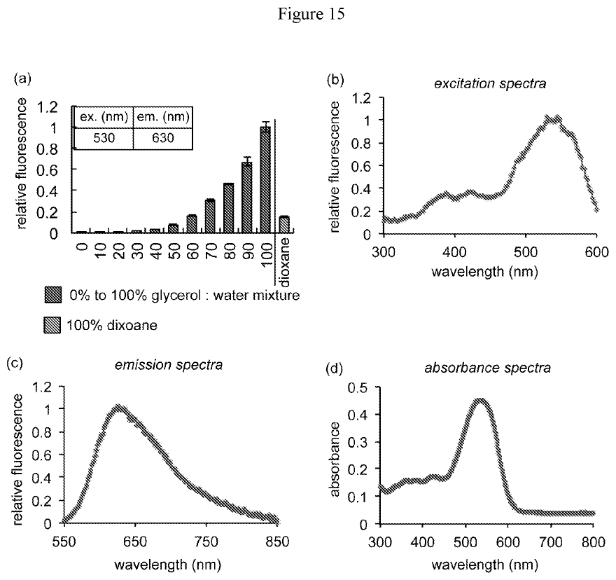

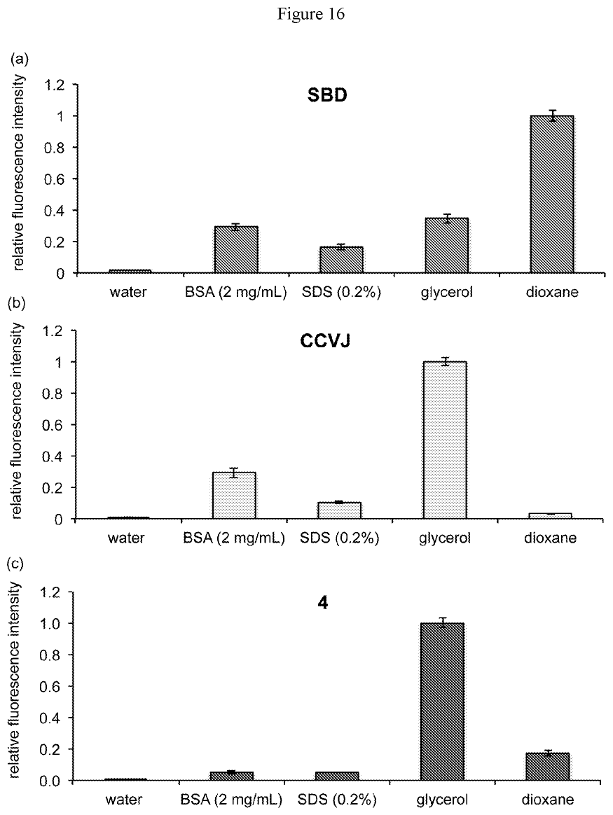

Using the AggTag method, the inventors expected that the fluorophore should remain dark when POI is folded. Aggregation of POI, however, should bury the fluorophore in protein aggregates that can restrict its rotation and trigger fluorescence (FIG. 4a). 4 bears two additional hydroxyl groups to improve solubility of 3 and a warhead for bioconjugation to Halo-Tag (FIG. 4b). The fluorescent properties of 4 were found to be identical to 3 (FIG. 15). In addition, 4 exhibited substantially lower fluorescent background signal than typical solvatochromic fluorophores (e.g., SBD) and molecular rotor fluorophores (e.g. CCVJ), when incubated with bovine serum albumin as a representation of proteins with hydrophobic surfaces and sodium dodecyl sulfate as a mimic of lipids (FIG. 16).

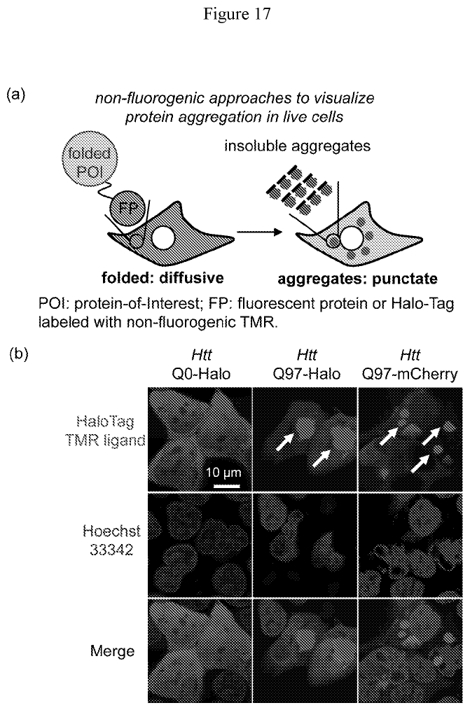

To test whether 4 is fluorogenic upon protein aggregation in live cells, an experiment was conducted using the Huntingtin exon 1 protein (Htt) with expansion of a polyglutamine tract within its N-terminal domain, well known for its severe protein aggregation. The inventors first observed protein aggregation using conventional non-fluorogenic methods (FIG. 17a). When HEK293T cells expressed either Htt-97Q-mCherry or Htt-97Q-Halo conjugated with an always-fluorescent TMR ligand, the inventors identified obvious fluorescent punctate structures with diffuse background fluorescence (FIG. 17b). Cells expressing the non-aggregating Htt-OQ-Halo in conjugation with TMR exhibited only diffuse fluorescence (FIG. 17b).

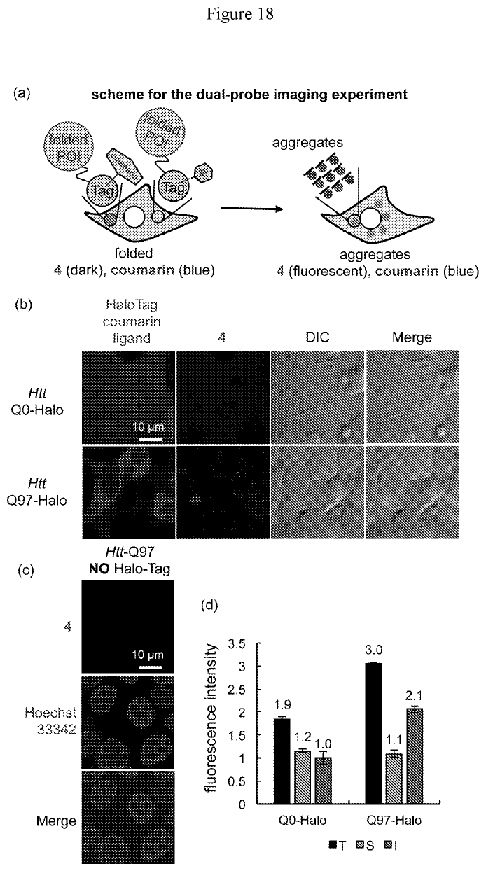

Thus, protein aggregation is primarily judged by the appearance of fluorescent puncta in non-fluorogenic methods (FIG. 17a). The AggTag method, however, yielded a dark background in cells expressing Htt-0Q-Halo labeled by 4 (FIG. 4c). The lack of fluorescence in these cells did not come from the lack of protein expression indicated by a dual-probe labeling experiment using 4 and an always-fluorescent coumarin ligand (FIGS. 18a-b). The turn-on fluorescence from 4, however, was observed as puncta without any background diffuse fluorescence when Htt-97Q-Halo was expressed (FIGS. 4c and 18b).

This fluorescence was not due to non-specific binding of 4 in cells, because HEK293T cells expressing Htt-97Q without Halo-Tag showed no fluorescent signal (FIG. 18c). In accordance with these observations, fluorescence from cell lysates confirmed that the fluorescence of Htt-97Q-Halo was more intense than that of Htt-0Q-Halo (FIGS. 4d and 18d). Furthermore, fractionation of the lysate indicated that the fluorescence of Htt-97Q-Halo primarily originated from the insoluble fraction (FIGS. 4d and 18d). Taken together, these experiments establish the AggTag method as a fluorogenic approach to detect protein aggregation in live cells.

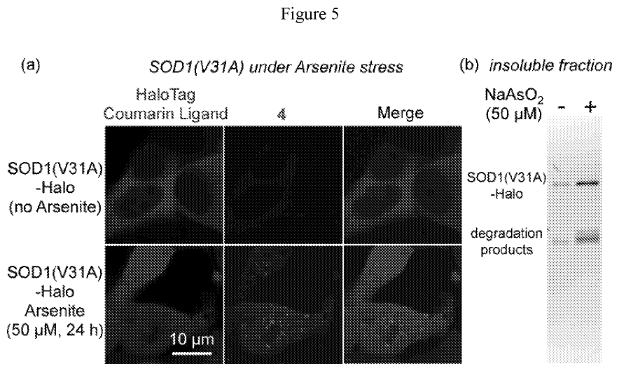

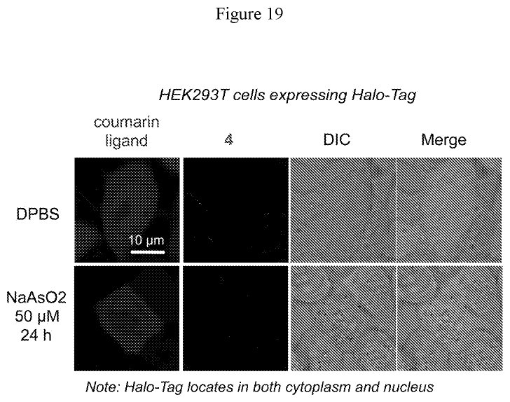

Experiments further demonstrated that the AggTag method could visualize previously invisible misfolded soluble proteins in live cells. The SOD1(V31A) mutant is associated with a slow disease progression. So far, little had been known about its aggregation propensity in live cells. To this end, the inventors expressed and labeled SOD(V31A)-Halo fusion protein simultaneously with the coumarin ligand and 4 in HEK293T cells. Using the coumarin fluorescence, the inventors found that SOD1(V31A) was primarily located in the cytoplasm and the oxidative stress inducer NaAsO₂ induced the partial translocation of SOD1(V31A) to the nucleus. Although these observations are consistent to previously reported cellular locations of other SOD1 mutants, it was unclear whether NaAsO₂ caused aggregation because the coumarin fluorescence remained diffuse before and after stress (left panel, FIG. 5a). By contrast, 4 was dark before stress but exhibited both diffuse and punctate fluorescent structures in stressed cells (middle panel, FIG. 5a). This turn-on fluorescence signal was not due to aggregation of the Halo-4 conjugate, as it remained dark in stressed cells (FIG. 19).

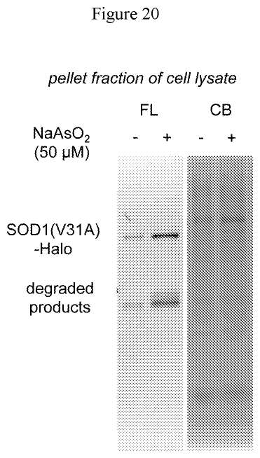

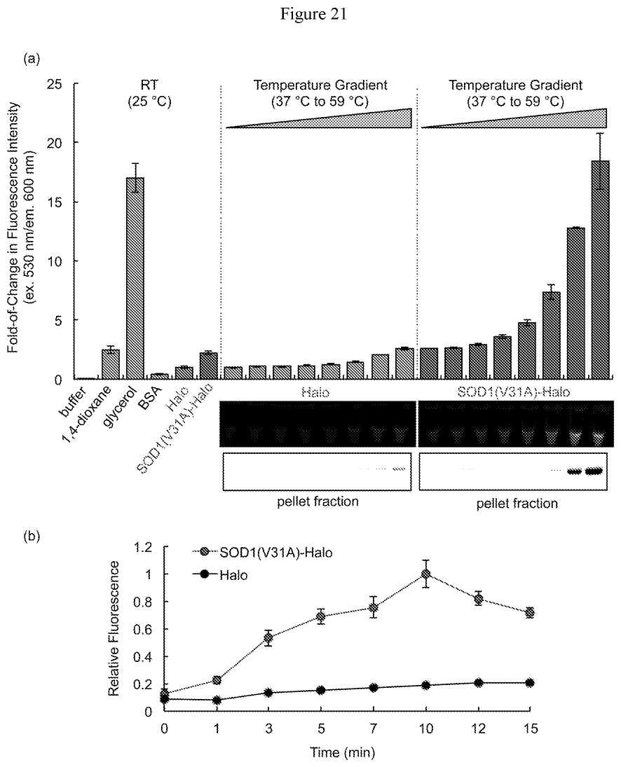

The punctate fluorescence could be rationalized by a fractionation experiment wherein more aggregates of SOD1(V31A) were found in cells treated with NaAsO₂ (FIGS. 5b and 20). The diffusive fluorescence of 4 was believed to arise from misfolded soluble proteins, as demonstrated by several lines of evidence from in vitro heat-induced aggregation experiments. First, the inventors found that fluorescence of the SOD1(V31A)-Halo-4 conjugate increased in the absence of insoluble protein aggregates (FIG. 21a). The Halo-4 conjugate, as a control, remained non-fluorescent under all heat conditions (FIG. 21). Second, the kinetics of fluorescence increase was much faster than the kinetics of turbidity (FIG. 22a). Finally, the kinetics of fluorescence coincided with the kinetics of protein misfolding (FIG. 22b). Collectively, these data support the belief that the AggTag method in combination with 4 is able to selectively visualize both misfolded soluble proteins and insoluble aggregates via diffuse and punctate fluorescence, respectively, in live cells.

Cellular Background and Control Experiments of Oligomer-Detecting Compounds.

A series of control experiments were carried out to demonstrate that the turn-on fluorescence specifically monitors misfolding of POIs. First, 4 fluorescence was not observed in cells expressing Halo-Tag in the presence of the NaAsO₂ stressor (50 M, 18 h, 37° C.). Second, HEK293T cells co-expressing Htt-Q97-GFP and Halo-Tag or just expressing Htt-Q97 showed no fluorescence of 4. Third, turn-on fluorescence was not observed in cells co-expressing Halo-Tag and SOD1-V31A-GFP in the presence of the NaAsO₂ stressor (50 μM, 18 h, 37° C.). These control experiments collectively demonstrate that the observed fluorescent signal from 4 was not due to non-specific binding between 4 and aggregates of other cellular proteins in cells.

Compounds Detecting Insoluble Aggregates in Test Tubes (AggTag Method).

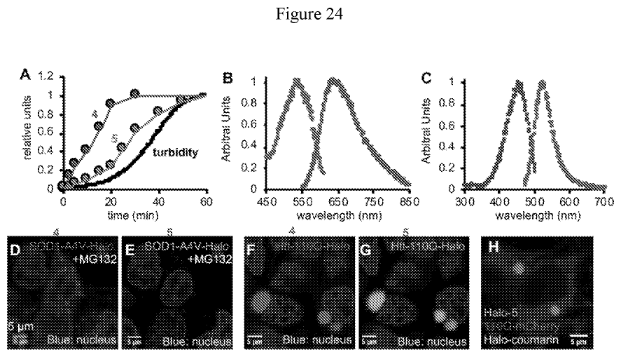

2 only activates fluorescence when POI forms insoluble aggregates, while it remains dark when POI is folded and forms soluble oligomers. The inventors installed Halo-tag reactive warhead to 2, resulting in 5. In heat-induced aggregation of SOD1-A4V-Halo that is conjugated to 5, it was found that the kinetics of P2b fluorescence increase was similar to that of turbidity whose signal originates from insoluble aggregates (green vs black curve, FIG. 24a). In contrast, when SOD1 A4V-Halo was conjugated with 5, the kinetics of 4 fluorescence increase was faster than that of turbidity and fluorescence increase of P2b (red vs black curve, FIG. 24a). 5 exhibits a quantum yield of 0.24 in insoluble aggregates and molar extinction coefficient of 23,716 M⁻¹·cm⁻¹, about 40% of EGFP (=0.6; c=56,000 M⁻¹·cm⁻¹).

Compounds Detecting Insoluble Aggregates of Specific Proteins in Live Cells (AggTag Method).

It is important to distinguish insoluble aggregates from misfolded oligomers, because they do not only have distinct functions in cells, but also are managed differently by cells.^{46,47} Soluble oligomers are targeted to the Juxta-Nuclear Quality (JUNQ) control compartment, which forms under severe stress conditions and contains polyubiquitylated proteins, such as mutants of SOD1. In contrast, insoluble aggregates, such as polyglutamine-expanded Huntingtin (Htt-polyQ), are sequestered into a cytoplasmic compartment known as the Insoluble Protein Deposit (IPOD). Thus, misfolded oligomers do not necessarily display as a diffusive structure, instead they can reside in granular structures that appear to be almost identical to granules formed by insoluble aggregates.

The spectra of 4 (Eₓ/Eₘ=530/600 nm; FIG. 24e) and 5 (Eₓ/Eₘ=450/520 nm; FIGS. 24b-c) make them suited for two-color imaging applications, wherein the fluorescence of 4 (red) and 5 (green) can monitor misfolded oligomers and insoluble aggregates, respectively (FIGS. 24b-c).

Combination of 4 and 5 enables a two-color imaging strategy to differentiate insoluble aggregates from soluble oligomers. The inventors carried out live cell imaging experiments, wherein 4 or 5 was used to visualize aggregation of POI-Halo fusion proteins in HEK293T cells. Under proteasome inhibition by a drug MG132, mutants of SOD1 has been shown to form JUNQ compartments that contain soluble oligomers.⁴⁷ If this were true, it would be expected that granules exhibit turn-on fluorescence with 4 but not 5. To this end, the inventors labeled HEK293T cells expressing SOD1-A4V-Halo simultaneously with 4 and 5 (both at 0.5 μM) for 24 h and treated cells with 5 μM MG132 for 8 h. As expected, SOD1 A4V-Halo formed mostly perinuclear granules that only exhibited red fluorescence from 4 (FIG. 24d) and dark fluorescence to a background level from 5 (FIG. 24d). The inventors further carried out experiments using Htt-PolyQ with 110 glutamine repeats (Htt-110Q-Halo). Htt-polyQ with longer than 78Q forms IPOD inclusions that contain insoluble aggregates at both cytosolic and perinuclear localizations.⁴⁷ Consistent to this note, Htt-110Q-Halo formed inclusions that exhibit fluorescence from both 4 and 5 (FIGS. 24f-g).

Cellular Background and Control Experiments of Insoluble Aggregates-Detecting Compounds.

As a control, 5 fluorescence was not observed in cells co-expressing Halo-tag and Htt-110Q-mChery (FIG. 24h), suggesting that observed fluorescent signal from 5 was not due to non-specific binding between 5 and aggregates of other cellular proteins in cells.

Compounds Enable Bioconjugation to Halo-Tag and SNAP-Tag Fusion Proteins (AggTag Method).

Halo-Tag is an engineered dehalogenase that reacts with chloroalkane molecules to form stable covalent enzyme-ligand conjugates, and it serves as an ideal sensor platform because it exhibits fast labeling kinetics, a bioorthogonal reaction profile, and demonstrated evolvability^{48,49}. SNAP-tag is a prominent self-labelling protein tag used for live cell imaging of POIs, due to its relatively small size (19.4 kDa, two-thirds the size of GFP as 27 kDa) and fast labelling kinetics.⁵⁰⁻⁵³

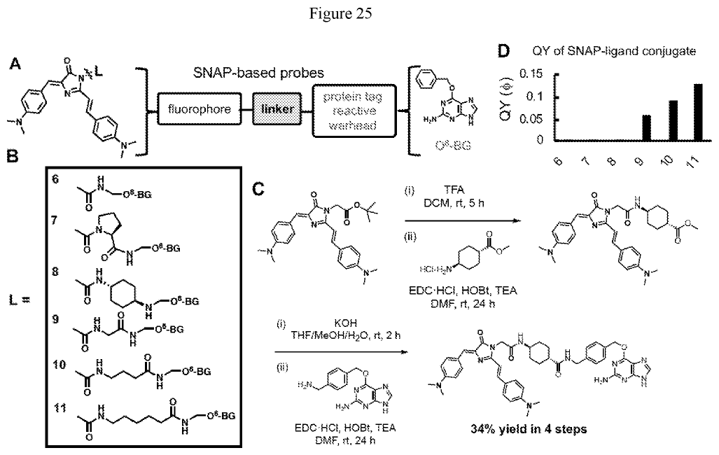

Probes can be developed to detect aggregation of POI fused with SNAP-tag. Using 3 as the fluorophore (FIG. 25a), 6-11 were synthesized by conjugating six different types of linkers (sarcosine, proline, cyclohexane, glycine, propane, and hexane; FIG. 25b) to 3 and O⁶-BG. Synthesis is described in FIG. 25c and produces the final product with a 34% yield. When conjugated with folded SNAP-Tag, 6-8 with short or rigid linkers exhibited minimal fluorescence increase (ϕ<0.01; FIG. 25d) and 9-11 with long and flexible linkers resulted in fluorescently bright conjugates (ϕ>0.06; FIG. 25d). These results support that 6-8 can be used for SNAP-Tag fusion proteins.

Experimental Methods

Plasmids. Mammalian expression: pHTN vector (Promega, Inc) with a stop codon added to the c-terminal of Halo-Tag protein. The SOD-1 gene was amplified from the pF146 pSOD1WTAcGFP1 (a gift from Elizabeth Fisher, Addgene plasmid #26407), respectively. The V31A mutation was introduced to SOD1 via QuickChange PCR. The Htt-97Q gene was amplified from the pCDNA3.1-Htt-97Q-mCherry (Max Planck Institute of Biochemistry). These genes were sub-cloned into a pHTC HaloTag CMV-neo vector by the PIPE cloning method. Protein expression: pET29b vectors were constructed to encode Halo-Tag-His6, SOD1(V31A)-linker-Halo-His6 (linker contains a TEV protease cleavage site), and α-synuclein.

Protein expression and purification. Halo-Tag and SOD1(V31A)-Halo: E. coli BL21 DE3* competent cells harboring a pBAD vector encoding σ32-I54N were transformed with pET29b vectors containing Halo-His6 and SOD1(V31A)-TEV-Halo-His6 proteins. Expression and purification was carried out as previously described. In brief, cells expressing recombinant proteins were thawed and lysed by sonication at 4° C. in the presence of a protease inhibitor (1 mM PMSF). Lysed cells were centrifuged for 60 min at 16,000×g. The supernatant was collected and loaded onto a 6 mL BioRad Nuvia Ni-IMAC column and washed with 120 mL of buffer containing 50 mM Tris·HCl (pH 7.5) and 100 mM NaCl. The protein was then eluted by gradient addition of buffer containing 50 mM Tris·HCl(pH 7.5), 100 mM NaCl, and 500 mM imidazole over a volume of 48 mL. The protein fractions were identified by SDS-PAGE analysis, pooled, and concentrated. The protein was further purified using a 120 mL HiPrep™ 16/60 Sephacryl™ S-200 HR size-exclusion column. The protein containing fractions were identified by SDS-PAGE gel analysis, pooled, and concentrated. No significant impurities were identified and purity was estimated to be >98% based on SDS-PAGE. SOD1(V31A): Purified SOD1(V31A)-Halo protein was subjected to a 1 h TEV protease cleavage (0.50 μM TEV protease for every 10 μM SOD1-Halo protein) in the presence of 1 mM DTT at 25° C. Reaction mixture was retro-purified via BioRad Nuvia Ni-IMAC resin. Flow through was collected as cleaved SOD1(V31A). No significant impurities were identified and Halo purity was estimated to be >98% based on SDS-PAGE. α-synuclein: Protein expression was induced with 0.1 M IPTG, 5 hours, 37° C. Harvest culture at 5,000 RPM, 15 min, 4° C. Resuspend the pellet in DPBS (200 mg/L KCl, 200 mg/L KH₂PO₄, 8 g/L NaCl, 2.16 g/L Na₂HPO₄.7H₂O), transfer to a 50 mL conical tube. Spin at 4,000 rpm, 30 min, 4° C. Decant the supernatant, and store the pellet in a −80° C. freezer. Next morning, resuspend defrosted pellet in osmotic shock buffer (30 mM Tris-HC, 40% sucrose, 2 mM EDTA, pH 7.5; 100 mL for each liter of starter culture). Incubate for 10 min at room temperature. Collect pellet by centrifugation (12,000 rpm, 20 min). Quickly resuspend pellet with ice-cold water (90 mL for each liter of starter culture). Add 4 M MgCl₂ (76.5 μl for each liter of starting culture) and keep on ice for 3 min. Centrifuge at 15,000 rpm, 30 min. Collect and filter supernatant. Add 2 M Tris-HC, pH 8 at a 1:100 (v/v) ratio. Load supernatant onto UNO Sphere Q column (5 mL, Bio-Rad), elute with a gradient from Buffer A (20 mM Tris, pH 8) to Buffer B (20 mM Tris, 500 mM NaCl, pH 8). Analyze fractions by 15% SDS-PAGE gel. Pool protein-containing fractions, and dilute by half with degassed Buffer A. Centrifuge at 15,000 rpm, 30 min to remove aggregates. Load onto an EnrichQ column (8 mL) on the NGC-Quest10 FPLC system and elute with a gradient from 0% to 100% Buffer B. Analyze fractions by 15% SDS-PAGE gel. Pool protein-containing fractions and dilute by half with degassed Buffer A. Load onto an EnrichQ column (8 mL) and elute with a gradient from 40% to 52% Buffer B. Analyze fractions by 15% SDS-PAGE gel. Pool protein containing fractions and flash freeze small aliquots of protein. Proteins can only be thawed once before use.

Confocal microscope imaging. The HEK293T cells were seeded at 25% confluency 24 h prior to transfection in poly-D-lysine coated 35 mm glass bottom dishes (MatTek Corporation). Cells were grown in DMEM media supplemented with 10% FBS and Penicillin-Streptomycin antibiotics until they reached 50-60% confluency. Transfection was carried out using X-tremeGene™ 9 DNA transfection reagent (Roche) according to the manufacturer's instructions. Proteins were expressed for 24 h prior to analyses. To label proteins with Halo-Tag fusion, protein expression was carried out in the presence of 1 μM 4 or 1 μM TMR Halo-Tag ligand to form covalent conjugate with the Halo-Tag domain. For a dual-probe labeling scheme, 1 μM 4 and 1 μM coumarin Halo-Tag ligand to simultaneously form covalent conjugate with the Halo-Tag domain.

To wash off unbound Halo ligands, the cells were washed extensively by replacing media with fresh DMEM and incubating for 30 min at 37° C. For confocal fluorescence imaging with either 4-labeled Halo fusion, TMR-labeled Halo fusion, EGFP fusion, or mCherry fusion proteins, DMEM media was replaced with FluoroBrite™ DMEM media (ThermoFisher) supplemented with 10% FBS, and Hoechst 33342 (0.1 μg/mL). For confocal fluorescence imaging with either dual-probe labeled Halo fusion with 4 and coumarin ligand, DMEM media was replaced with FluoroBrite™ DMEM media (ThermoFisher) supplemented with 10% FBS. The samples were incubated for 30 min prior to imaging. Media was replaced with fresh FluoroBrite™ DMEM media (ThermoFisher) supplemented with 10% FBS prior to imaging. Confocal images were obtained using Olympus FluoView™ FV1000 confocal microscope. The EGFP fluorescence was visualized using blue argon (488 nm) laser. Nuclear staining and coumarin fluorescence was visualized using violet laser (405 nm). Fluorescence of TMR and 4 were visualized using green HeNe laser (543 nm).

Chemical-Induced Proteome Stress and Confocal Imaging of Stressed Cells.

HEK293T cultures were seeded at 25% confluency 24 h prior to transfection in 12-well plate for time dependent fluorescence plate reader analysis or 35 mm glass bottom culture dishes (Poly-d-lysine coated, MatTek Corporation). Cells were grown in DMEM medium supplemented with 10% FBS and penicillin-streptomycin antibiotics until they reached 50-60% confluency. Transfection was performed using X-tremeGene™ 9 DNA transfection reagent (Roche). After 24 h of protein expression and co-translational labeling, medium was replaced with fresh DMEM to diffuse out unbound ligands. After 30 min, media was replaced by fresh DMEM medium containing DMSO vehicle or NaAsO₂ (50 μM). Cells were incubated for 18 h, 37° C. in a CO₂ incubator. Nuclear stain Hoechst 33342 (0.1 g/mL) was added to the medium 30 min prior to confocal imaging. Aggregation Assays. α-synuclein: Aggregation solution contained 70 or 140 μM α-synuclein in 20 mM HEPES (pH 7.5) and 100 mM NaCl. 10 μM of 3 or ThT was added to the solution at the beginning of reaction. Previous studies have shown that the kinetics of α-synuclein aggregation is unaffected by the addition of ThT. A volume of 150 μL of the mixture was pipetted into a well of clear-bottom 96-well plate, which was subsequently sealed with Mylar plate sealers. The plate was loaded on a Heidolph vibrating platform shaker and shake at 1,350 rpm at 37° C. At indicated time points, fluorescence reading was obtained using a Tecan M1000Pro fluorescence plate reader. Fluorescence intensity was recorded at Eₓ=530 nm/Eₘ=600 nm for 3 and Eₓ=450 nm/Eₘ=480 nm for ThT. Each sample was run in triplicate or quadruplicate. SOD1(V31A): Aggregation was carried out with DPBS buffer containing indicated concentrations of EDTA that is used to chelate the structural metal of SOD1. Aggregation solution contained 42 μM SOD1(V31A) or SOD1(V31A)-Halo and 21 M of 3 was added to the solution at the beginning of reaction. Reaction was carried out quiescently at indicated temperatures. Fluorescence of 3 was measured at Eₓ=530 nm/Eₘ=600 nm.

X-ray structure determination of 3. Single crystals of 3 (C₂₈H₃₄N₄O₃) were grown by evaporation in EtOH and ethyl acetate mixed solvents. A suitable crystal was selected and mounted on a nylon loop, with help of paratone oil on a ‘SMART APEX CCD area detector’ diffractometer (molybdenum target, 1600 kW). The crystal was kept at 243 K during the data collection. Using Olex2⁵⁴, the structure was solved with the XS⁵⁵ structure solution program using Direct Methods and refined with the XL refinement package using Least Squares minimization without use of any constraints/restraints. The hydrogen atoms were placed geometrically, and rode their parent atoms during the refinement. Crystal Datafor C₂₈H₃N₄O₃ (M=474.59 g/mol): orthorhombic, space group P2₁2₁2₁ (no. 19), a=9.895(4) Å, b=21.470(10) Å, c=25.711(12) Å, V=5463(4) Å³, Z=8, T=243 K, μ(MoKα)=0.076 mm⁻¹, Dcalc=1.154 g/cm³, 51829 reflections measured (3.7°≤2Θ≤57.88°), 13915 unique (Rᵢₙₜ=0.1226, R_{sigma}=0.1327) which were used in all calculations. The final R₁ was 0.0570 (>2sigma(I)) and wR₂ was 0.1767 (all data).

Negative staining electron microscopy. Carbon-coated 400 mesh EM grids supported by Formvar film were plasma-discharged prior to use. A small amount (˜5 L) of sample suspension was applied onto the grids. The grids were then blotted to remove excess of the sample and stained with 1% aqueous uranyl. Images were collected at indicated resolutions.

### SUMMARY

Embodiments as reported herein demonstrate that analogues of FP chromophores can fluoresce in protein aggregates. Different from previous non-fluorogenic methods, methods and compositions, as reported herein, can visualize both misfolded soluble proteins and insoluble aggregates in intact live cells. Such fluorogenic detection can be achieved via chemical modulation of fluorophores with molecular rotor and AIE properties, providing new applications for this large family of molecules. The unique fluorogenicity of this class of probes, combined with the AggTag method, make them generally applicable to a wide range of proteins whose aggregation is associated with diseases and suited to potentiate screening platform to explore therapeutics that can ameliorate aggregation of these pathogenic proteins.

It will be apparent to those skilled in the art that numerous modifications and variations of the described examples and embodiments are possible in light of the above teachings of the disclosure. The disclosed examples and embodiments are presented for purposes of illustration only. Other alternative embodiments may include some or all of the features of the various embodiments disclosed herein. Therefore, it is the intent to cover all such modifications and alternative embodiments as may come within the true scope of this invention, which is to be given the full breadth thereof. Additionally, the disclosure of a range of values is a disclosure of every numerical value within that range, including the end points.

Therefore, while certain exemplary embodiments of apparatuses and methods of making and using the same have been discussed and illustrated herein, it is to be distinctly understood that the invention is not limited thereto but may be otherwise variously embodied and practiced within the scope of the following claims. Documents reported herein are incorporated by reference herein; however, if there is a conflict between this document and the incorporated document, this document controls.

The following documents may be interesting or useful in reviewing and appreciating embodiments as reported herein. Inclusion of these documents is not an admission of any sort.
Grimm, et al., A general method to improve fluorophores for live-cell and single-molecule microscopy. Nat Methods 2015, 12 (3), 244-50, 3 p following 250.
Ando, et al., An optical marker based on the UV-induced green-to-red photoconversion of a fluorescent protein. Proc Natl Acad Sci USA 2002, 99 (20), 12651-6; Subach, et al., Chromophore transformations in red fluorescent proteins. Chem Rev 2012, 112 (7), 4308-27; Tomura, et al., Monitoring cellular movement in vivo with photoconvertible fluorescence protein “Kaede” transgenic mice. Proc Natl Acad Sci USA 2008, 105 (31), 10871-6.
Hong, et al., Aggregation-induced emission. Chem Soc Rev 2011, 40 (11), 5361-88.
Lashuel, et al., The many faces of alphα-synuclein: from structure and toxicity to therapeutic target. Nat Rev Neurosci 2013, 14 (1), 38-48.
Qin, et al., Highly sensitive amyloid detection enabled by thioflavin T dimers. Mol BioSys 2010, 6 (10), 1791-5.
Rosen, et al., Mutations in Cu/Zn superoxide dismutase gene are associated with familial amyotrophic lateral sclerosis. Nature 1993, 362 (6415), 59-62.
Dangoumau, et al., Novel SOD1 mutation p.V31A identified with a slowly progressive form of amyotrophic lateral sclerosis. Neurobiol Aging 2014, 35 (1), 266 e1-4.
Los, et al., HaloTag: a novel protein labeling technology for cell imaging and protein analysis. ACS Chem Biol 2008, 3 (6), 373-82; Lin, et al., A Generalizable platform for interrogating target- and signal-specific consequences of electrophilic modifications in redox-dependent cell signaling. J Am Chem Soc 2015, 137 (19), 6232-6244; Ballister, et al., Localized light-induced protein dimerization in living cells using a photocaged dimerizer. Nat Commun 2014, 5, 5475.
Gidalevitz, et al., Progressive disruption of cellular protein folding in models of polyglutamine diseases. Science 2006, 311 (5766), 1471-4; DiFiglia, et al., Aggregation of huntingtin in neuronal intranuclear inclusions and dystrophic neurites in brain. Science 1997, 277 (5334), 1990-3.
Ramdzan, et al., Tracking protein aggregation and mislocalization in cells with flow cytometiy. Nat Methods 2012, 9 (5), 467-476; Kitamura, et al., Conformational Analysis of Misfolded Protein Aggregation by FRET and live-cell imaging techniques. IntJMolSci 2015, 16 (3), 6076-6092.
Mateju, et al., An aberrant phase transition of stress granules triggered by misfolded protein and prevented by chaperone function. EMBO J 2017, 36 (12), 1669-1687; Zhong, et al., Nuclear export of misfolded SOD1 mediated by a normally buried NES-like sequence reduces proteotoxicity in the nucleus. Elfe 2017, 6.
Haidekker, et al., Molecular rotors—fluorescent biosensors for viscosity and flow. Org Biomol Chem 2007, 5(11), 1669-1678; (b) Qian, H; Cousins, M E.; Horak, E. H; Wakefield, A.; Liptak, M D.; Aprahamian, I., Suppression of Kasha's rule as a mechanism for fluorescent molecular rotors and aggregation-induced emission. Nat Chem 2017, 9 (1), 83-87.
Kalaparthi, et al., The nature of ultrabrightness of nanoporous fluorescent particles with physically encapsulated fluorescent dyes. J Mater Chem C 2016, 4 (11), 2197-2210.
Wurth, et al., Relative and absolute determination of fluorescence quantum yields of transparent samples. Nat Protoc 2013, 8 (8), 1535-1550.
Dolomanov, et al., OLEX2: a complete structure solution, refinement and analysis program. J Appl Crystallogr 2009, 42, 339-341.
Sheldrick, G. M., A short history of SHELX. Acta Crystallogr A 2008, 64, 112-122.
Baldridge, et al., Efficient synthesis of new 4-aiylideneimidazolin-5-ones related to the GFP chromophore by 2+3 cyclocondensation of arylideneimines with imidate ylides, Synthesis 2010, 14, 2424-2436.

### REFERENCES

The following references are each relied upon and incorporated herein in their entirety.
[1] Balch, W. E., Morimoto, R. I., Dillin, A., and Kelly, J. W. (2008) Adapting proteostasis for disease intervention, Science 319, 916-919.
[2] Eisele, Y. S., Monteiro, C., Fearns, C., Encalada, S. E., Wiseman, R. L., Powers, E. T., and Kelly, J. W. (2015) Targeting protein aggregation for the treatment of degenerative diseases, Nature reviews. Drug Discovery 14, 759-780.
[3] Labbadia, J., and Morimoto, R. I. (2013) Huntington's disease: underlying molecular mechanisms and emerging concepts, Trends Biochem Sci 38, 378-385.
[4] Kaushik, S., and Cuervo, A. M. (2015) Proteostasis and aging, Nature Medicine 21, 1406-1415.
[5] Olzscha, H., Schermann, S. M., Woerner, A. C., Pinkert, S., Hecht, M. H., Tartaglia, G. G., Vendruscolo, M., Hayer-Hartl, M., Hart, F. U., and Vabulas, R. M. (2011) Amyloid-like aggregates sequester numerous metastable proteins with essential cellular functions, Cell 144, 67-78.
[6] Hipp, M. S., Park, S. H., and Hart, F. U. (2014) Proteostasis impairment in protein-misfolding and -aggregation diseases, Trends in Cell Biology 24, 506-514.
[7] Powers, E. T., Morimoto, R. I., Dillin, A., Kelly, J. W., and Balch, W. E. (2009) Biological and chemical approaches to diseases of proteostasis deficiency, Annu Rev Biochem 78, 959-991.
[8] Chiti, F., and Dobson, C. M. (2006) Protein misfolding, functional amyloid, and human disease, Annu Rev Biochem 75, 333-366.
[9] Aguzzi, A., and O'Connor, T. (2010) Protein aggregation diseases: pathogenicity and therapeutic perspectives, Nature Reviews. Drug Discovery 9, 237-248.
[10] Jensen, M. B., and Jasper, H. (2014) Mitochondrial proteostasis in the control of aging and longevity, Cell Metabolism 20, 214-225.
[11] Kayed, R., Head, E., Thompson, J. L., McIntire, T. M., Milton, S. C., Cotman, C. W., and Glabe, C. G. (2003) Common structure of soluble amyloid oligomers implies common mechanism of pathogenesis, Science 300, 486-489.
[12] Haass, C., and Selkoe, D. J. (2007) Soluble protein oligomers in neurodegeneration: lessons from the Alzheimer's amyloid beta-peptide, Nat Rev Mol Cell Biol 8, 101-112.
[13] Benilova, I., Karran, E., and De Strooper, B. (2012) The toxic Aβ oligomer and Alzheimer's disease: an emperor in need of clothes, Nat. Neurosci. 15, 349.
[14] Kim, Y. E., Hosp, F., Frottin, F., Ge, H., Mann, M., Hayer-Hartl, M., and Hartl, F. U. (2016) Soluble Oligomers of PolyQ-Expanded Huntingtin Target a Multiplicity of Key Cellular Factors, Molecular Cell 63, 951-964.
[15] Ross, C. A., and Poirier, M. A. (2005) What is the role of protein aggregation in neurodegeneration?, Nat. Rev. Mol. Cell Biol. 6, 891.
[16] Gupta, R., Kasturi, P., Bracher, A., Loew, C., Zheng, M., Villella, A., Garza, D., Hartl, F. U., and Raychaudhuri, S. (2011) Firefly luciferase mutants as sensors of proteome stress, Nat Methods 8, 879-884.
[17] Winkler, J., Seybert, A., Konig, L., Pruggnaller, S., Haselmann, U., Soujik, V., Weiss, M., Frangakis, A. S., Mogk, A., and Bukau, B. (2010) Quantitative and spatio-temporal features of protein aggregation in Escherichia coli and consequences on protein quality control and cellular ageing, EMBO J 29, 910-923.
[18] Hsieh, T. Y., Nillegoda, N. B., Tyedmers, J., Bukau, B., Mogk, A., and Kramer, G. (2014) Monitoring Protein Misfolding by Site-Specific Labeling of Proteins In Vivo, PloS one 9.
[19] Ignatova, Z., and Gierasch, L. M. (2004) Monitoring protein stability and aggregation in vivo by real-time fluorescent labeling, P Natl Acad Sci USA 101, 523-528.
[20] Ramdzan, Y. M., Polling, S., Chia, C. P. Z., Ng, I. H. W., Ormsby, A. R., Croft, N. P., Purcell, A. W., Bogoyevitch, M. A., Ng, D. C. H., Gleeson, P. A., and Hatters, D. M. (2012) Tracking protein aggregation and mislocalization in cells with flow cytometry, Nat Methods 9, 467-U476.
[21] Nath, S., Meuvis, J., Hendrix, J., Carl, S. A., and Engelborghs, Y. (2010)
Early aggregation steps in alphα-synuclein as measured by FCS and FRET: evidence for a contagious conformational change, Biophys J 98, 1302-1311.
[22] Wood, R. J., Ormsby, A. R., Radwan, M., Cox, D., Sharma, A., Vopel, T., Ebbinghaus, S., Oliveberg, M., Reid, G. E., Dickson, A., and Hatters, D. M. (2018) A biosensor-based framework to measure latent proteostasis capacity, Nat Commun 9, 287.
[23] Kitamura, A., Nagata, K., and Kinjo, M. (2015) Conformational Analysis of Misfolded Protein Aggregation by FRET and Live-Cell Imaging Techniques, Int JMol Sci 16, 6076-6092.
[24] Walker, C. L., Lukyanov, K. A., Yampolsky, I. V., Mishin, A. S., Bommarius, A. S., Duraj-Thatte, A. M., Azizi, B., Tolbert, L. M., and Solntsev, K. M. (2015) Fluorescence imaging using synthetic GFP chromophores, Curr Opin Chem Biol 27, 64-74.
[25] Tsien, R. Y. (1998) The green fluorescent protein, Annual Review of Biochemistry 67, 509-544.
[26] Zimmer, M. (2002) Green fluorescent protein (GFP): applications, structure, and related photophysical behavior, Chem Rev 102, 759-781.
[27] Subach, F. V., and Verkhusha, V. V. (2012) Chromophore transformations in red fluorescent proteins, Chem Rev 112, 4308-4327.
[28] Baranov, M. S., Lukyanov, K. A., Borissova, A. O., Shamir, J., Kosenkov, D., Slipchenko, L. V., Tolbert, L. M., Yampolsky, I. V., and Solntsev, K. M. (2012) Conformationally locked chromophores as models of excited-state proton transfer in fluorescent proteins, J Am Chem Soc 134, 6025-6032.
[29] Baldridge, A., Samanta, S. R., Jayaraj, N., Ramamurthy, V., and Tolbert, L.
M. (2011) Steric and electronic effects in capsule-confined green fluorescent protein chromophores, J Am Chem Soc 133, 712-715.
[30] Williams, D. E., Dolgopolova, E. A., Pellechia, P. J., Palukoshka, A., Wilson, T. J., Tan, R., Maier, J. M., Greytak, A. B., Smith, M. D., Krause, J. A., and Shustova, N. B. (2015) Mimic of the green fluorescent protein beta-barrel: photophysics and dynamics of confined chromophores defined by a rigid porous scaffold, J Am Chem Soc 137, 2223-2226.
[31] Carayon, C., Ghodbane, A., Gibot, L., Dumur, R., Wang, J., Saffon, N., Rols,
M. P., Solntsev, K. M., and Fey-Forgues, S. (2016) Conjugates of Benzoxazole and GFP Chromophore with Aggregation-Induced Enhanced Emission: Influence of the Chain Length on the Formation of Particles and on the Dye Uptake by Living Cells, Small 12, 6602-6612.
[32] Baldridge, A., Feng, S., Chang, Y. T., and Tolbert, L. M. (2011) Recapture of GFP chromophore fluorescence in a protein host, ACS Comb Sci 13, 214-217.
[33] Paige, J. S., Wu, K. Y., and Jaffrey, S. R. (2011) RNA mimics of green fluorescent protein, Science 333, 642-646.
[34] Filonov, G. S., Moon, J. D., Svensen, N., and Jaffrey, S. R. (2014) Broccoli: rapid selection of an RNA mimic of green fluorescent protein by fluorescence-based selection and directed evolution, J Am Chem Soc 136, 16299-16308.
[35] Warner, K. D., Sjekloca, L., Song, W., Filonov, G. S., Jaffrey, S. R., and Ferre-D′Amare, A. R. (2017) A homodimer interface without base pairs in an RNA mimic of red fluorescent protein, Nat Chem Biol 13, 1195-1201.
[36] Feng, G., Luo, C., Yi, H., Yuan, L., Lin, B., Luo, X., Hu, X., Wang, H., Lei, C., Nie, Z., and Yao, S. (2017) DNA mimics of red fluorescent proteins (RFP) based on G-quadruplex-confined synthetic RFP chromophores, Nucleic Acids Res 45, 10380-10392.
[37] Kellenberger, C. A., Wilson, S. C., Sales-Lee, J., and Hammond, M. C. (2013) RNA-based fluorescent biosensors for live cell imaging of second messengers cyclic di-GMP and cyclic AMP-GMP, JAm Chem Soc 135, 4906-4909.
[38] Tsien, R. Y., and Miyawaki, A. (1998) Seeing the machinery of live cells, Science 280, 1954-1955.
[39] Rodriguez, E. A., Campbell, R. E., Lin, J. Y., Lin, M. Z., Miyawaki, A.,
Palmer, A. E., Shu, X., Zhang, J., and Tsien, R. Y. (2017) The Growing and Glowing Toolbox of Fluorescent and Photoactive Proteins, Trends Biochem Sci 42, 111-129.
[40] Ando, R., Hama, H., Yamamoto-Hino, M., Mizuno, H., and Miyawaki, A. (2002) An optical marker based on the UV-induced green-to-red photoconversion of a fluorescent protein, Proc Nat Acad Sci USA 99, 12651-12656.
[41] Tomura, M., Yoshida, N., Tanaka, J., Karasawa, S., Miwa, Y., Miyawaki, A., and Kanagawa, O. (2008) Monitoring cellular movement in vivo with photoconvertible fluorescence protein “Kaede” transgenic mice, Proc Natl Acad Sci USA 105, 10871-10876.
[42] Lashuel, H. A., Overk, C. R., Oueslati, A., and Masliah, E. (2013) The many faces of alpha-synuclein: from structure and toxicity to therapeutic target, Nat Rev Neurosci 14, 38-48.
[43] Qin, L., Vastl, J., and Gao, J. (2010) Highly sensitive amyloid detection enabled by thioflavin T dimers, Molecular BioSystems 6, 1791-1795.
[44] Rosen, D. R., Siddique, T., Patterson, D., Figlewicz, D. A., Sapp, P., Hentati,
A., Donaldson, D., Goto, J., O'Regan, J. P., Deng, H. X., and et al. (1993) Mutations in Cu/Zn superoxide dismutase gene are associated with familial amyotrophic lateral sclerosis, Nature 362, 59-62.
[45] Dangoumau, A., Verschueren, A., Hammouche, E., Papon, M. A., Blasco, H., Cherpi-Antar, C., Pouget, J., Corcia, P., Andres, C. R., and Vourc'h, P. (2014) Novel SOD1 mutation p.V31A identified with a slowly progressive form of amyotrophic lateral sclerosis, Neurobiol Aging 35, 266 e261-264.
[46] Kaganovich, D., Kopito, R., and Frydman, J. (2008) Misfolded proteins partition between two distinct quality control compartments, Nature 454, 1088-U1036.
[47] Sontag, E. M., Vonk, W. I. M., and Frydman, J. (2014) Sorting out the trash: the spatial nature of eukaryotic protein quality control, Curr Opin Cell Biol 26, 139-146.
[48] Long, M. J. C., Poganik, J. R., and Aye, Y. (2016) On-Demand Targeting: Investigating Biology with Proximity-Directed Chemistry, J Am Chem Soc 138, 3610-3622.
[49] Lin, H. Y., Haegele, J. A., Disare, M. T., Lin, Q. S., and Aye, Y. (2015) A Generalizable Platform for Interrogating Target- and Signal-Specific Consequences of Electrophilic Modifications in Redox-Dependent Cell Signaling, J Am Chem Soc 137, 6232-6244.
[50] Keppler, A., Gendreizig, S., Gronemeyer, T., Pick, H., Vogel, H., and Johnsson, K. (2003) A general method for the covalent labeling of fusion proteins with small molecules in vivo, Nat Biotechnol 21, 86-89.
[51] Gautier, A., Juillerat, A., Heinis, C., Correa, I. R., Jr., Kindermann, M., Beaufils, F., and Johnsson, K. (2008) An engineered protein tag for multiprotein labeling in living cells, Chemistry & Biology 15, 128-136.
[52] Xue, L., Karpenko, I. A., Hiblot, J., and Johnsson, K. (2015) Imaging and manipulating proteins in live cells through covalent labeling, Nat Chem Biol 11, 917-923.
[53] Jung, K. H., Fares, M., Grainger, L. S., Wolstenholme, C. H., Hou, A., Liu, Y., and Zhang, X. (2018) A SNAP-tag fluorogenic probe mimicking the chromophore of the red fluorescent protein Kaede, Org Biomol Chem.
[54] Dolomanov, O. V., Bourhis, L. J., Gildea, R. J., Howard, J. A. K., and Puschmann, H. (2009) OLEX2: a complete structure solution, refinement and analysis program, J Appl Crystallogr 42, 339-341.
[55] Sheldrick, G. M. (2008) A short history of SHELX, Acta Crystallogr A 64, 112-122.

1. A compound of Formula I: 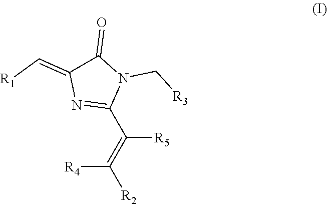
wherein;
R₁, R₂, and R₅ are independently selected from the moieties of Group 1, and R₄ is —H;
R₁, R₂, and R₄ are independently selected from the moieties of Group 1, and R₅ is —H;
R₁ and R₂ are independently selected from the moieties of Group 1, R₄ is —H, and R₅ is —H;
R₁ and R₂ are independently selected from the moieties of Group 1, R₄ is —H and R₅ is —CH₃;
R₁ and R₂ are independently selected from the moieties of Group 1, R₄ is —CH₃ and R₅ is —H; or
R₁, R₂, R₄, and R₅ are independently selected from the moieties of Group 1; and
wherein the moieties of Group 1 are selected from the group consisting of 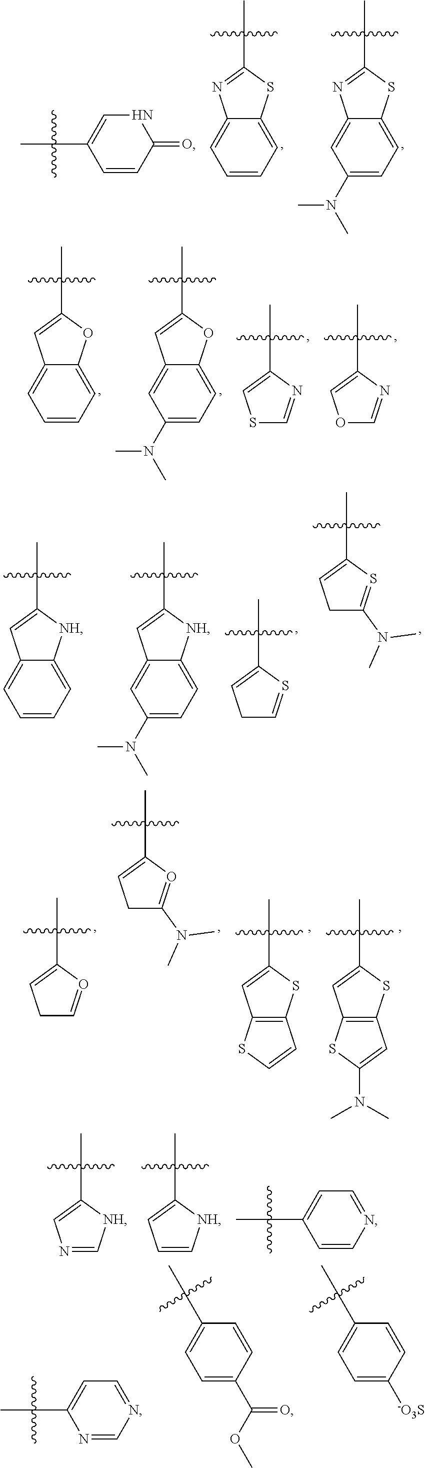 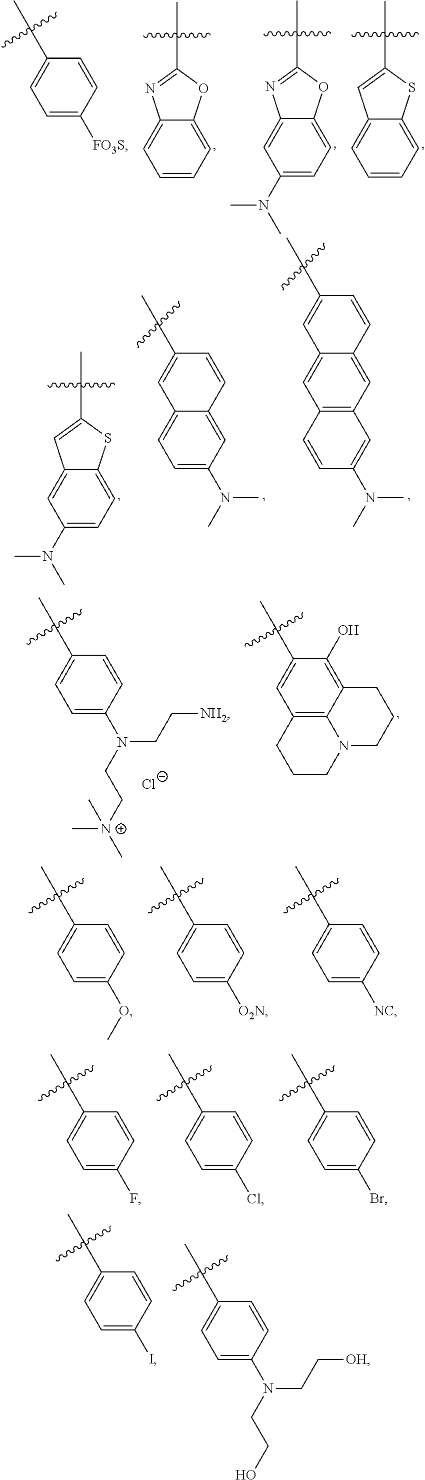 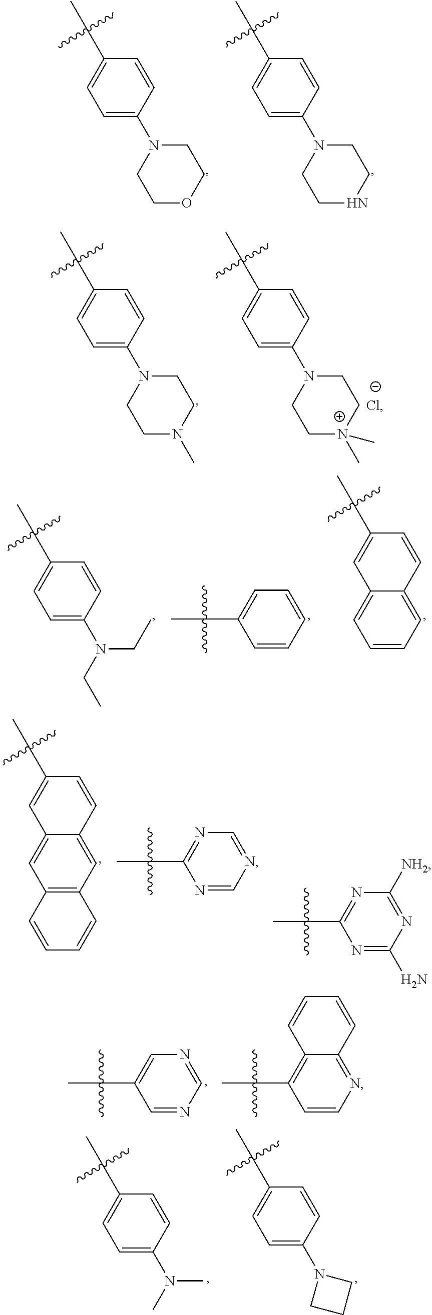 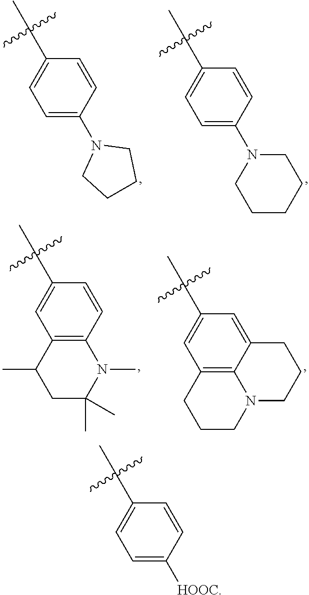
wherein substituents may be at para-, meta-, or ortho- positions on the aromatic ring, even if shown in only a single position above; and
wherein R₃ is a directing moiety that binds and bioconjugates at least one biological target, wherein R₃ is selected from the group consisting of: 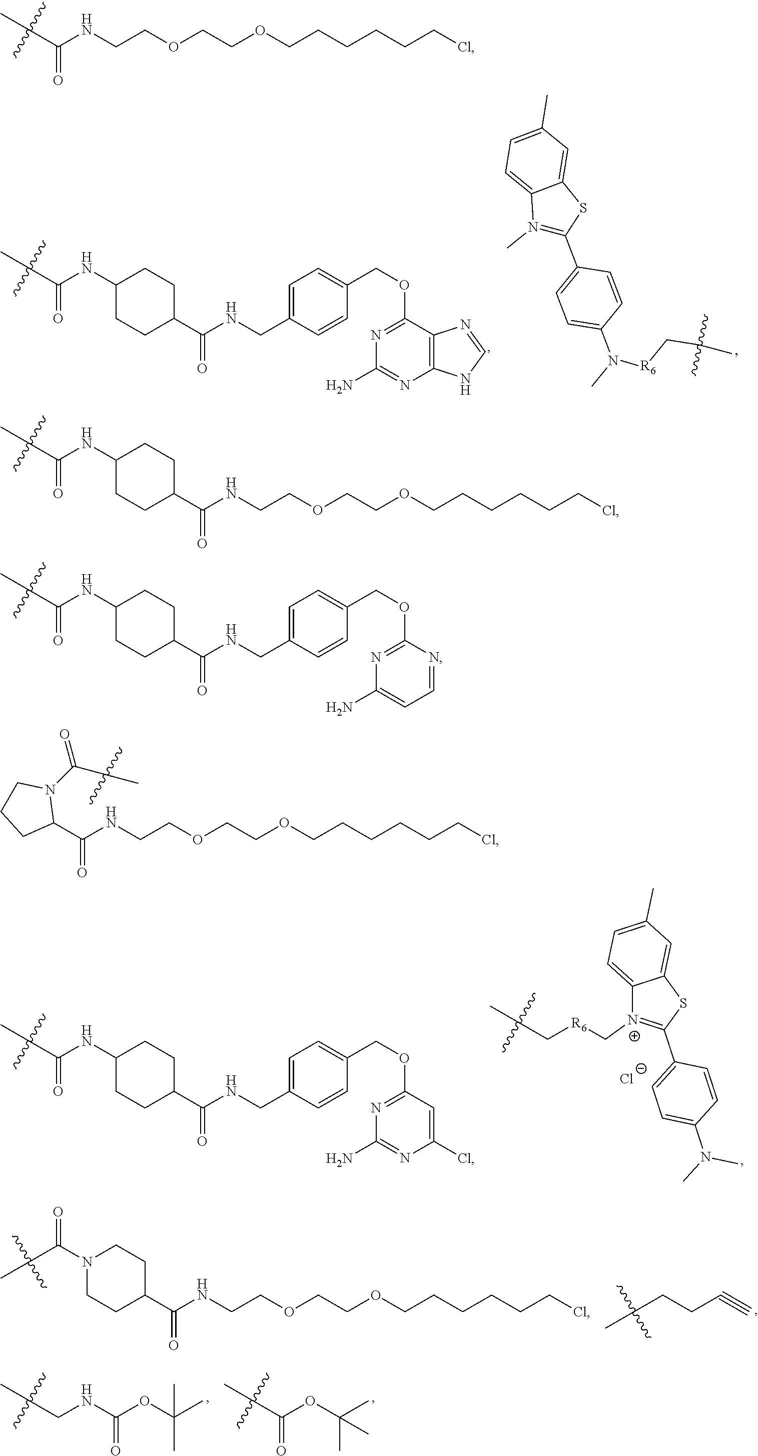 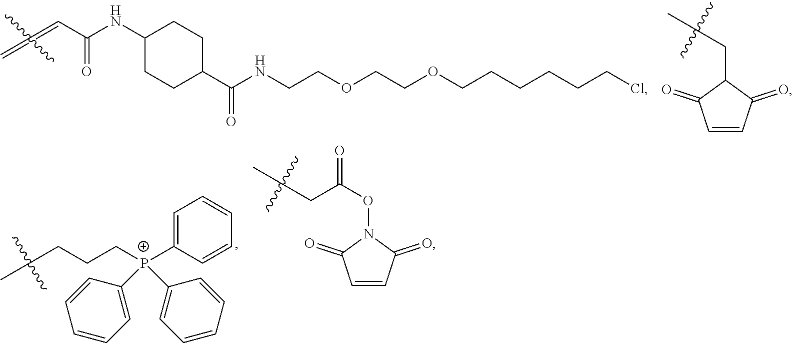
Wherein R₆ is selected from the group consisting of 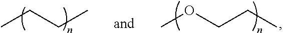
and wherein n is 0, 1, 2, 3, 4 or 5.

2. A compound of Formula II: 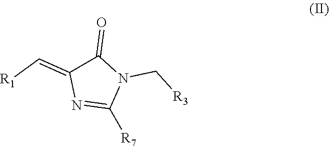
wherein
R₁ and R₇ are independently selected from a moiety of Group 1; or
R₁ is a moiety of Group 1, R₇ is a moiety of Group 2, and R₈ and R₉ are independently selected from —H, —CH₃, and a moiety of Group 1: wherein a moiety of Group 1 is selected from the 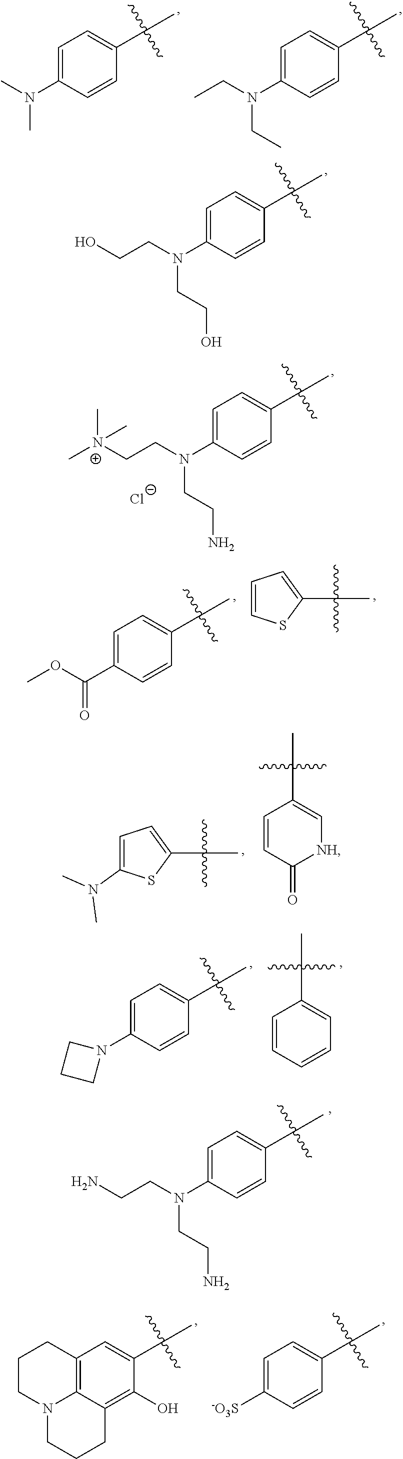 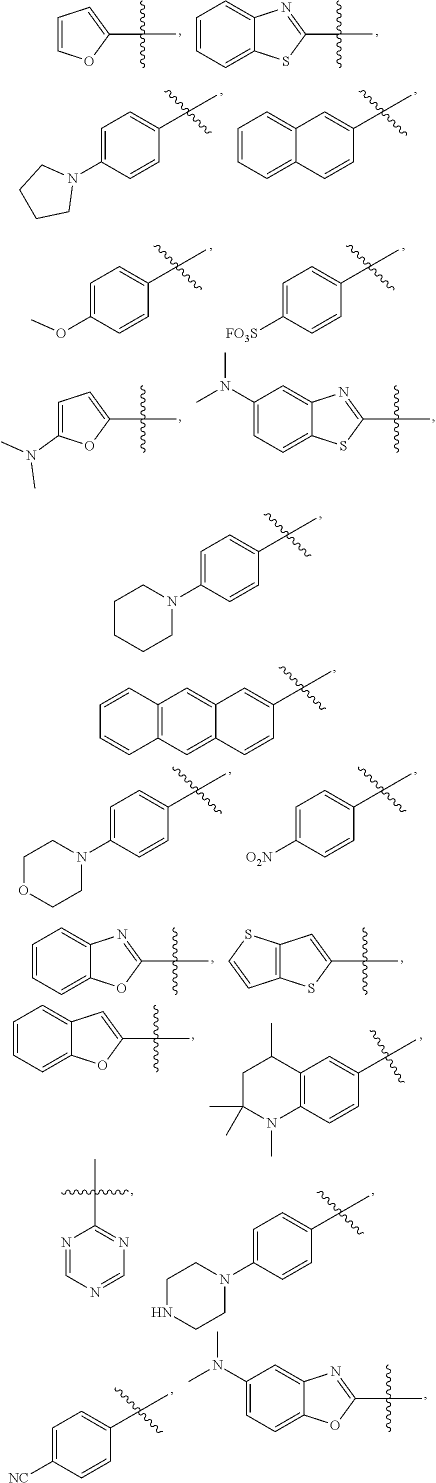 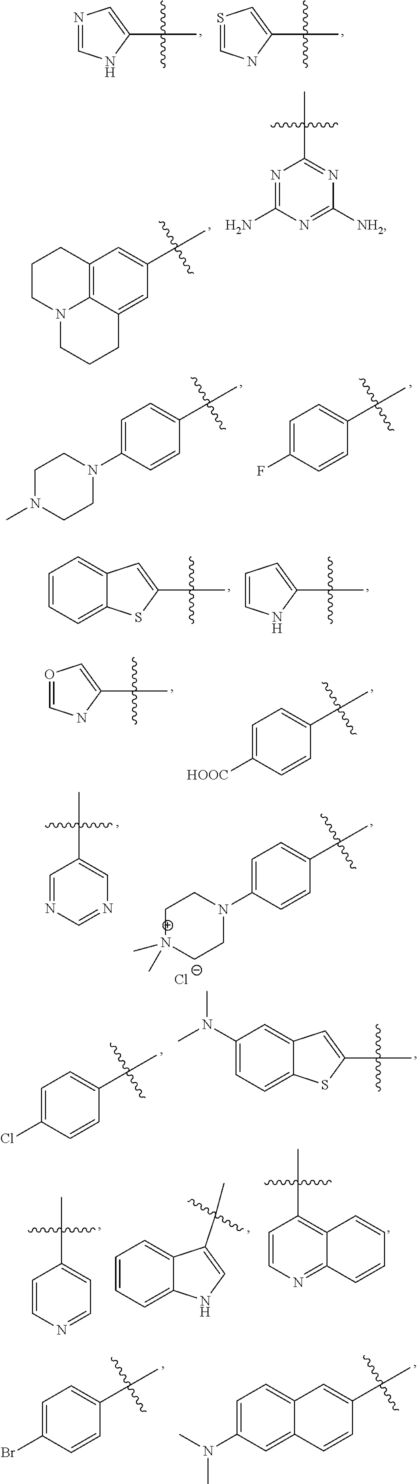 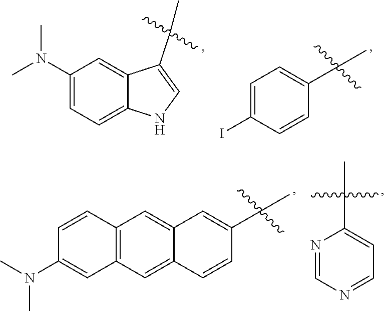 [SMILES: *c1c[nH]c2ccc(N(C)C)cc12 ; *c1ccc(I)cc1 ; *c1ccc2cc3cc(N(C)C)ccc3cc2c1 ; *c1ccncn1]
and wherein
a moiety of Group 2 is selected from the group consisting of 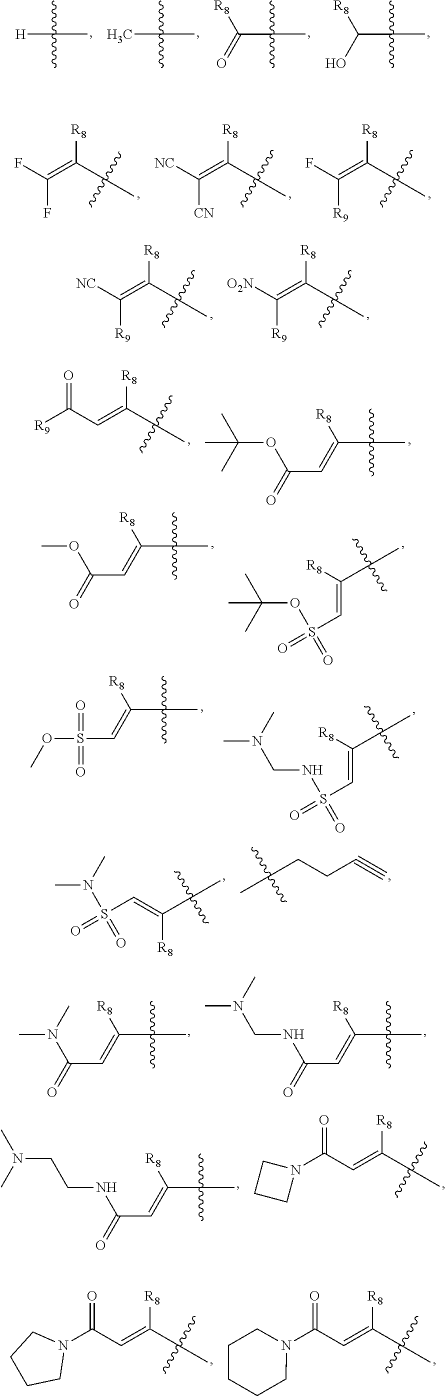 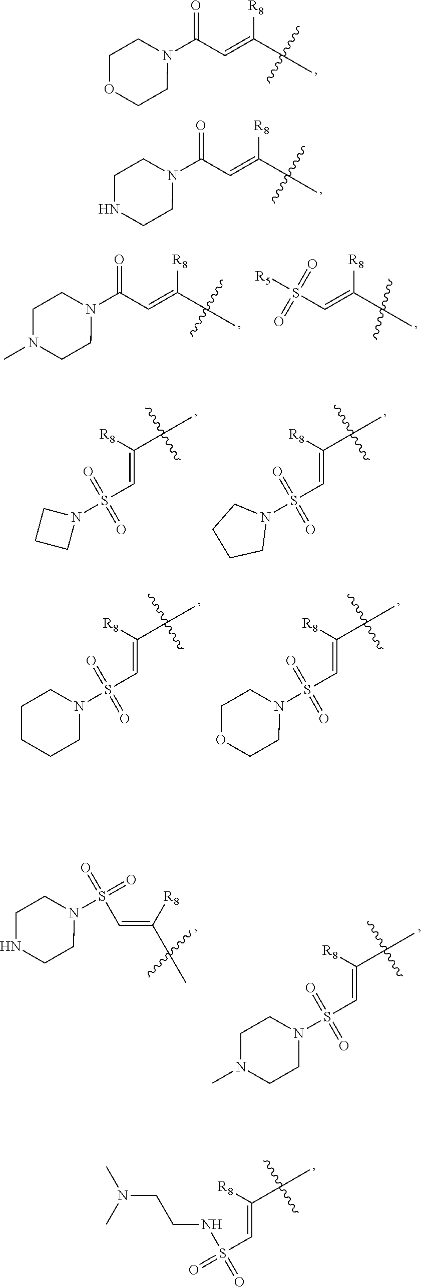
wherein substituents may be at para-, meta-, or ortho- positions on the aromatic ring of Group 1 and Group 2, even if shown in only a single position above; and wherein R3 is a directing moiety to bind and bioconjugate to biological targets, selected from the group consisting of: 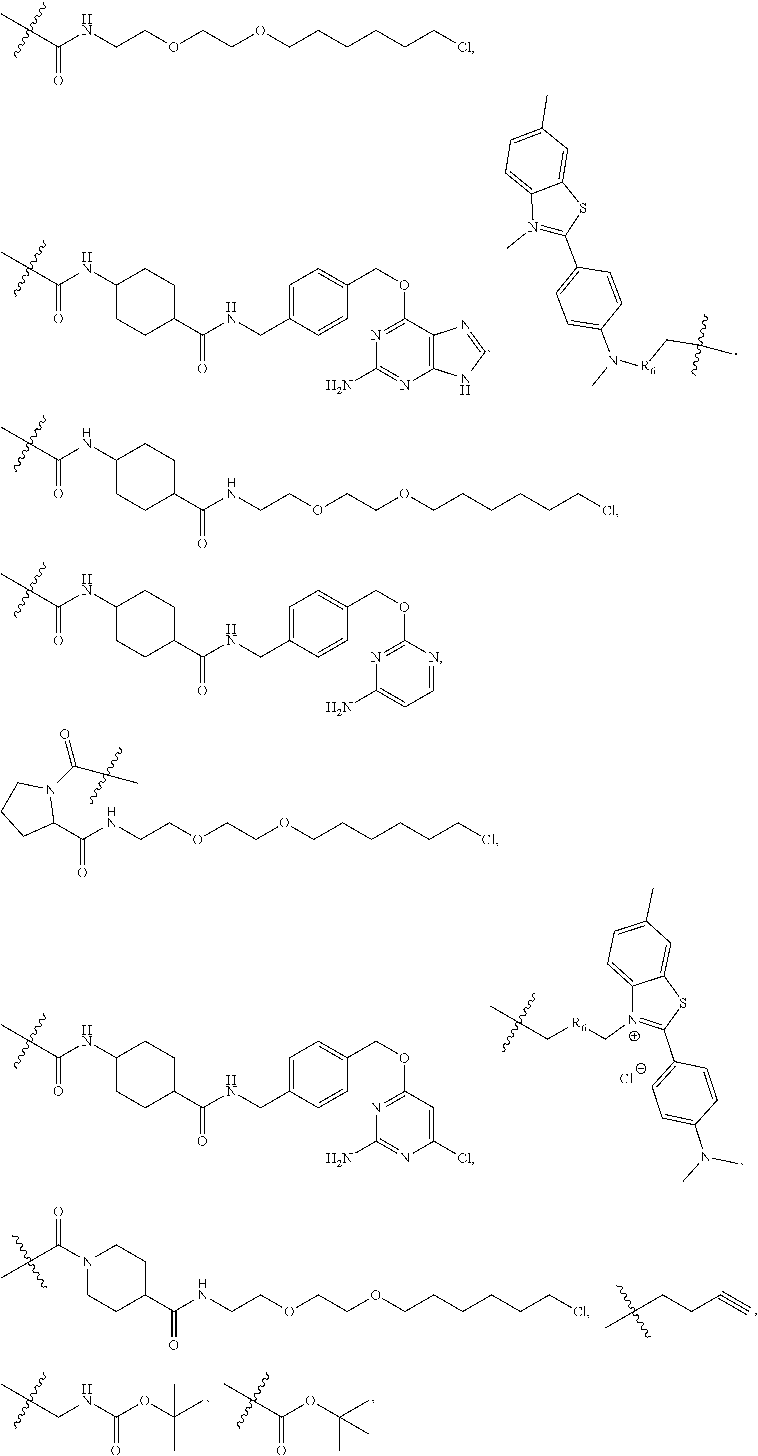 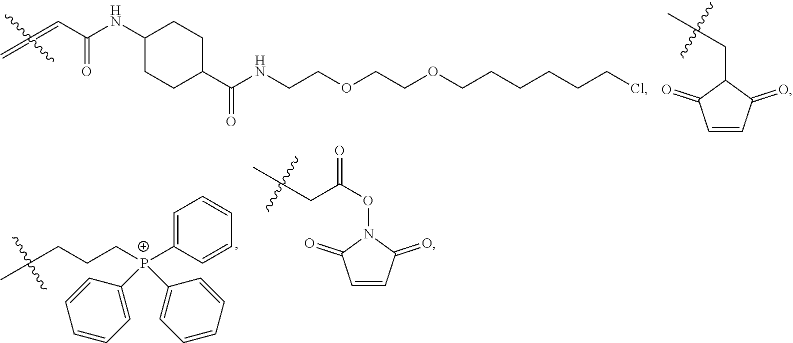
wherein, R₆ is selected from the group consisting of 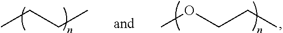
and wherein n is 0, 1, 2, 3, 4 or 5.

3. (canceled)

4. A method for detecting at least one of aggregated protein, misfolded protein, and amyloid fiber in a protein of interest, comprising:
performing a first measurement of fluorescence intensity of a protein of interest;
adding to the protein of interest a fluorescent protein chromophore; and
performing a second measurement of fluorescence intensity of the protein of interest; wherein increased fluorescence is indicative of at least one of aggregation, misfolding, and amyloid fiber in the protein of interest.

5. (canceled)

6. The method of claim 4, wherein the method is conducted in a cell in vivo.

7. The method of claim 4, wherein the method is conducted in vitro.

8. The method of claim 4, further comprising purifying the protein of interest prior to the first measurement.

9. The method of claim 4, wherein the fluorescent protein chromophore comprises a thioflavm-T guiding group or a tert-butyloxycarbonyl guiding group.

10. The method for detecting at least one of aggregated protein, misfolded protein, and amyloid fiber in a protein of interest of claim 4, said method comprising:
performing a first measurement of fluorescence intensity of a standard protein;
and
comparing the fluorescence intensity of the standard protein with the fluorescence intensity of the protein of interest, wherein increased fluorescence of the protein of interest relative to the fluorescence intensity of the standard protein is indicative of at least one of aggregation, misfolding, and amyloid fiber in the protein of interest.

11. A kit for detecting at least one of aggregated protein, misfolded protein, and amyloid fiber, comprising:
one or more fluorescent protein chromophores of claim 1 of a known concentration in a stock solution, one or more standard protein samples that form aggregated protein, misfolded protein, and amyloid fiber and optionally, instructions for use in detecting at least one of one of the aggregated protein, misfolded protein, and amyloid fiber.

12. The kit of claim 11 wherein the stock solution is dimethyl sulfoxide or ethanol.

13. A kit for detecting at least one of aggregated protein, misfolded protein, and amyloid fiber, comprising:
one or more fluorescent protein chromophores of claim 2 of a known concentration in a stock solution, one or more standard protein samples that form aggregated protein, misfolded protein, and amyloid fiber and optionally, instructions for use in detecting at least one of one of the aggregated protein, misfolded protein, and amyloid fiber.

14. The method of claim 4, wherein the fluorescent protein chromophore is a compound of Formula I: 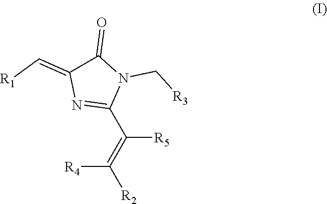
wherein;
R₁, R₂, and R₅ are independently selected from the moieties of Group 1, and R₄ is —H;
R₁, R₂, and R₄ are independently selected from the moieties of Group 1, and R₅ is —H;
R₁ and R₂ are independently selected from the moieties of Group 1, R₄ is —H, and R₅ is —H;
R₁ and R₂ are independently selected from the moieties of Group 1, R₄ is —H and R₅ is —CH₃;
R₁ and R₂ are independently selected from the moieties of Group 1, R₄ is —CH₃ and R₅ is —H; or
R₁, R₂, R₄, and R₅ are independently selected from the moieties of Group 1; and
wherein the moieties of Group 1 are selected from the group consisting of 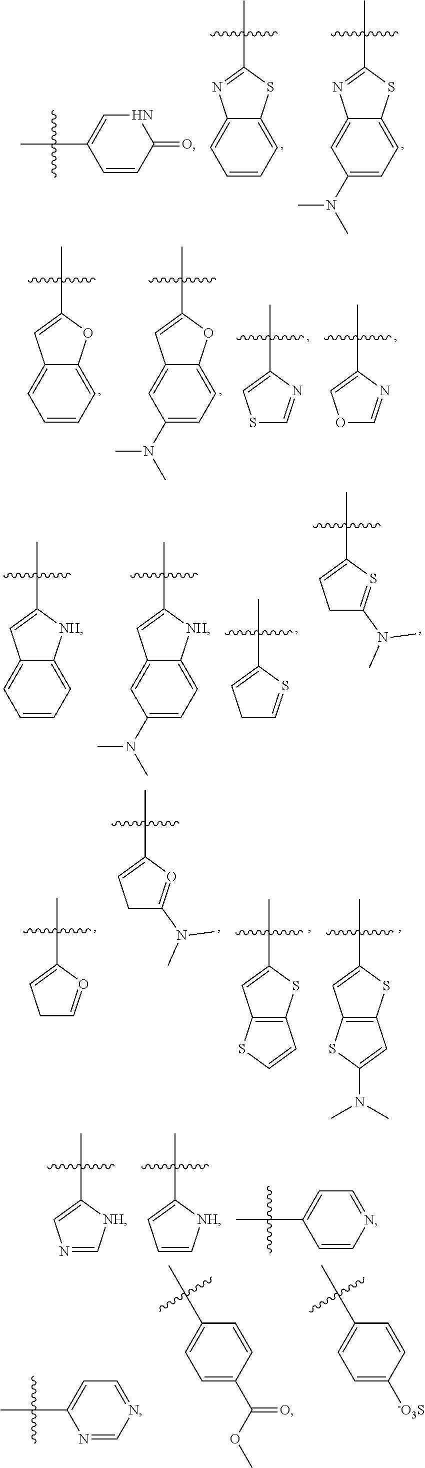 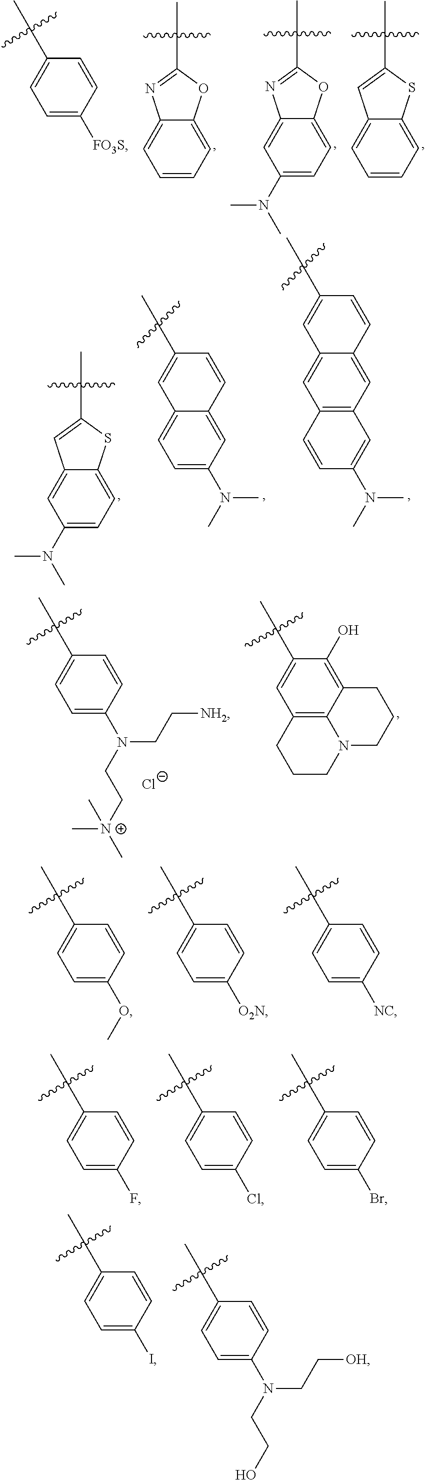 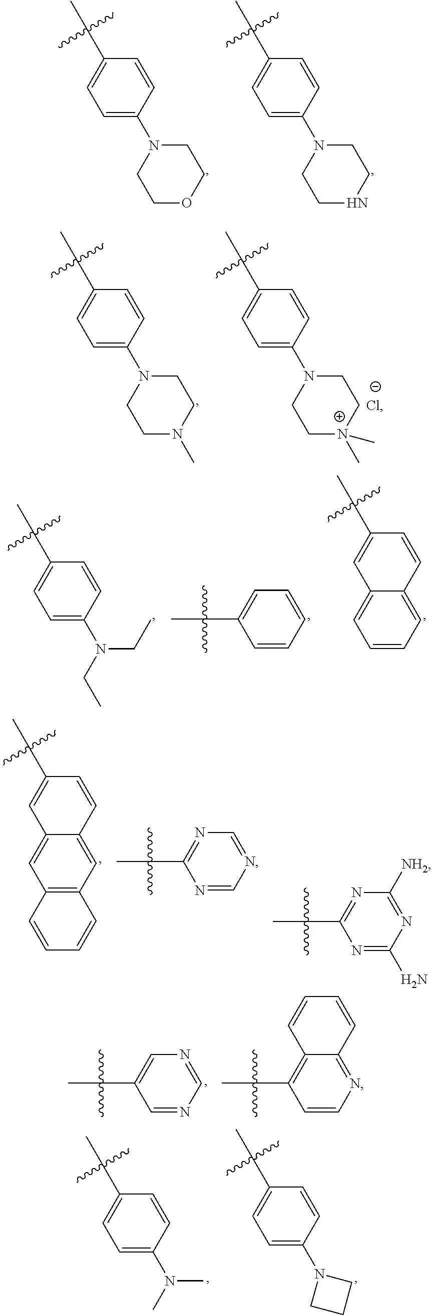 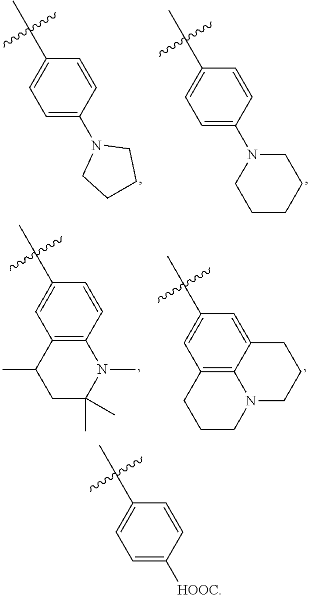
wherein substituents may be at para-, meta-, or ortho- positions on the aromatic ring, even if shown in only a single position above; and
wherein R₃ is a directing moiety that binds and bioconjugates at least one biological target, wherein R₃ is selected from the group consisting of: 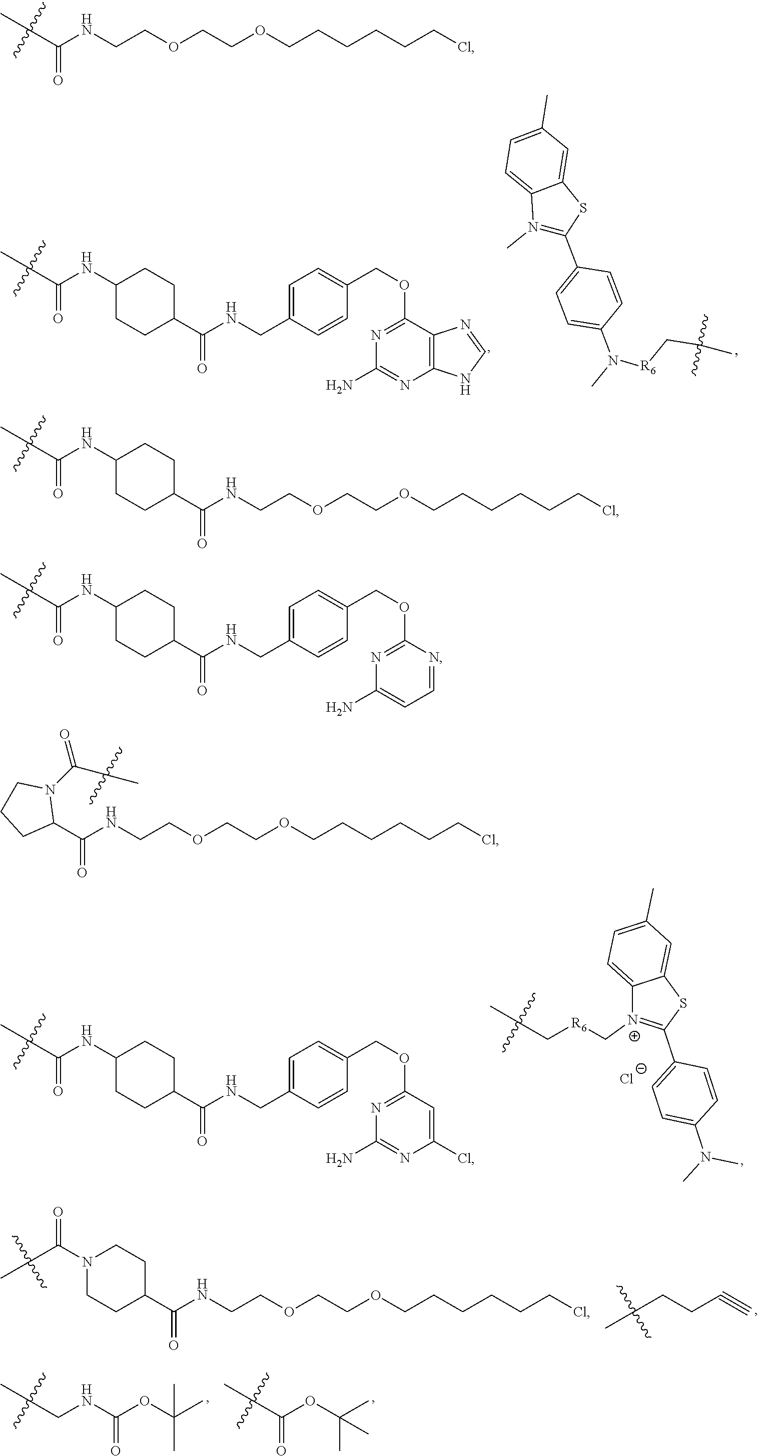 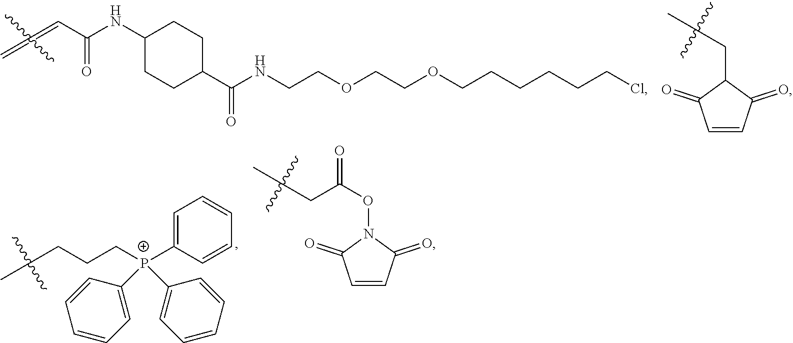
Wherein R₆ is selected from the group consisting of 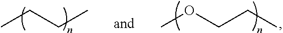
and wherein n is 0, 1, 2, 3, 4 or 5.

15. The method of claim 4, wherein the fluorescent protein chromophore is a compound of Formula II: 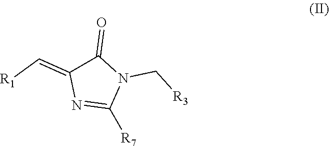
wherein
R₁ and R₇ are independently selected from a moiety of Group 1; or
R₁ is a moiety of Group 1, R₇ is a moiety of Group 2, and R₈ and R₉ are independently selected from —H, —CH₃, and a moiety of Group 1; wherein a moiety of Group 1 is selected from the group consisting of: 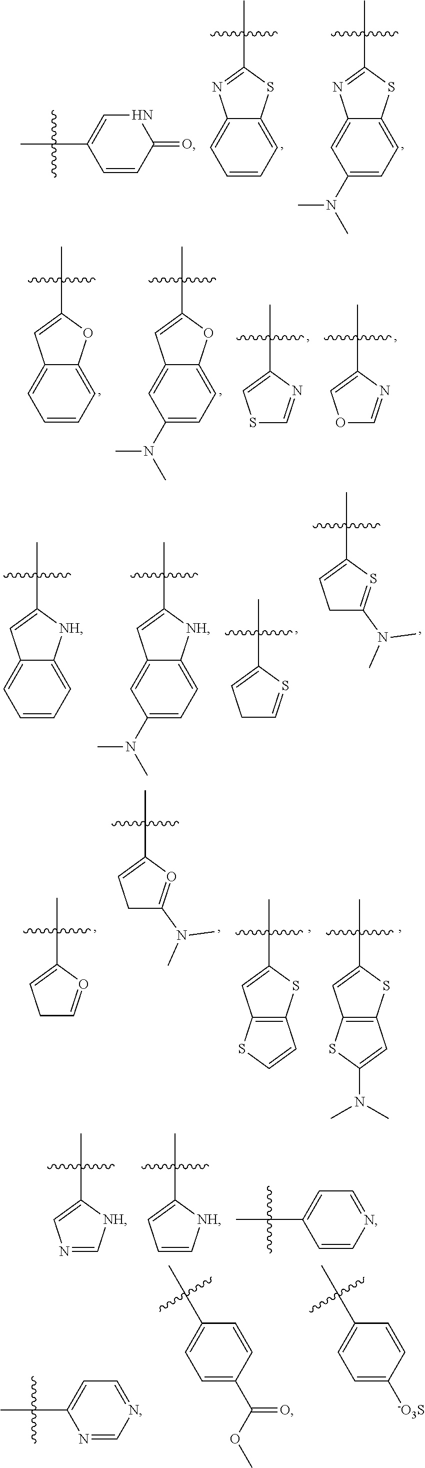 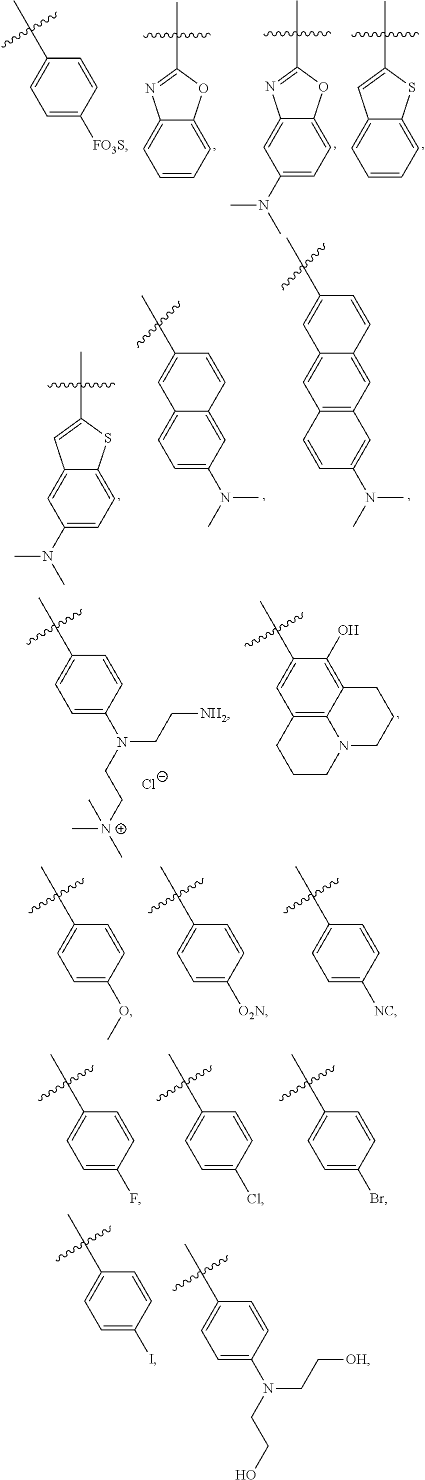 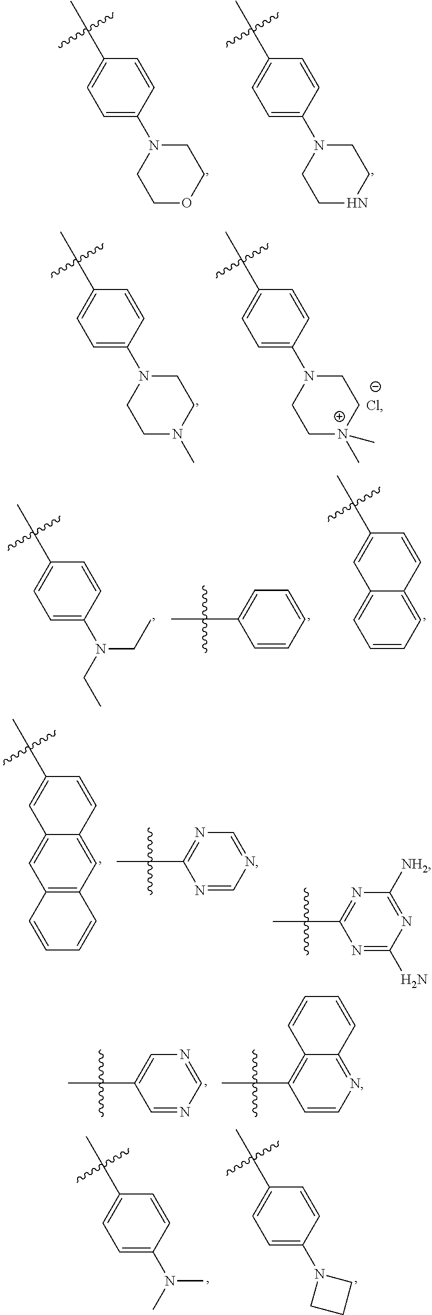 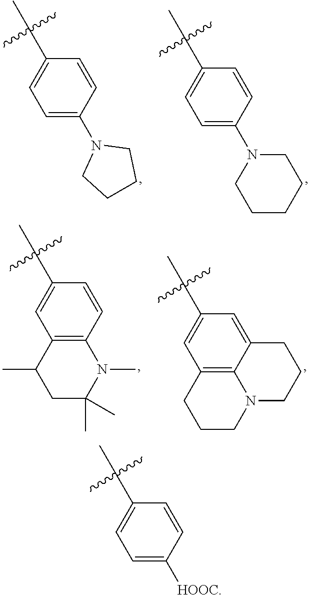
and wherein
a moiety of Group 2 is selected from the group consisting of 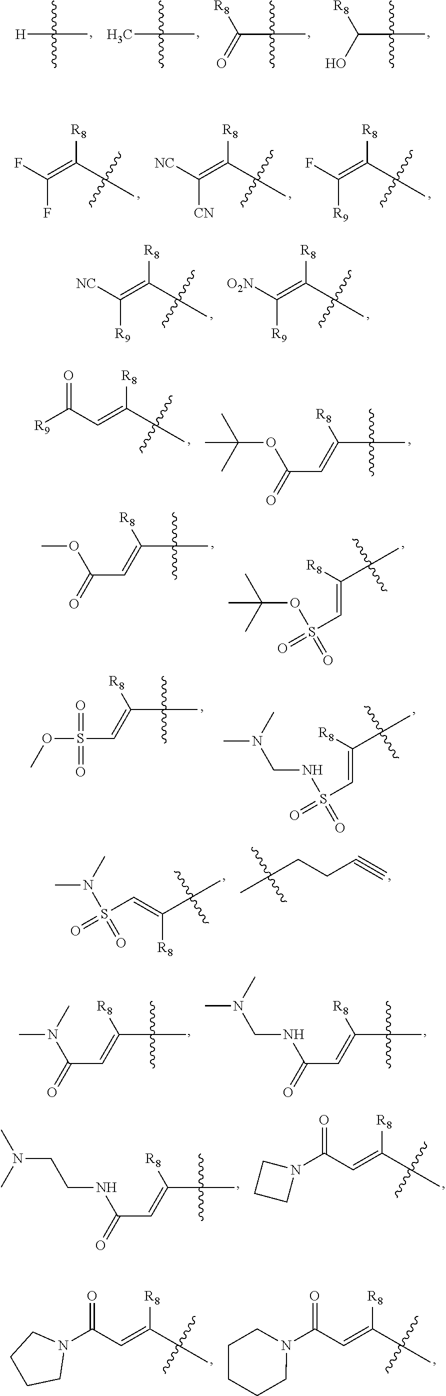 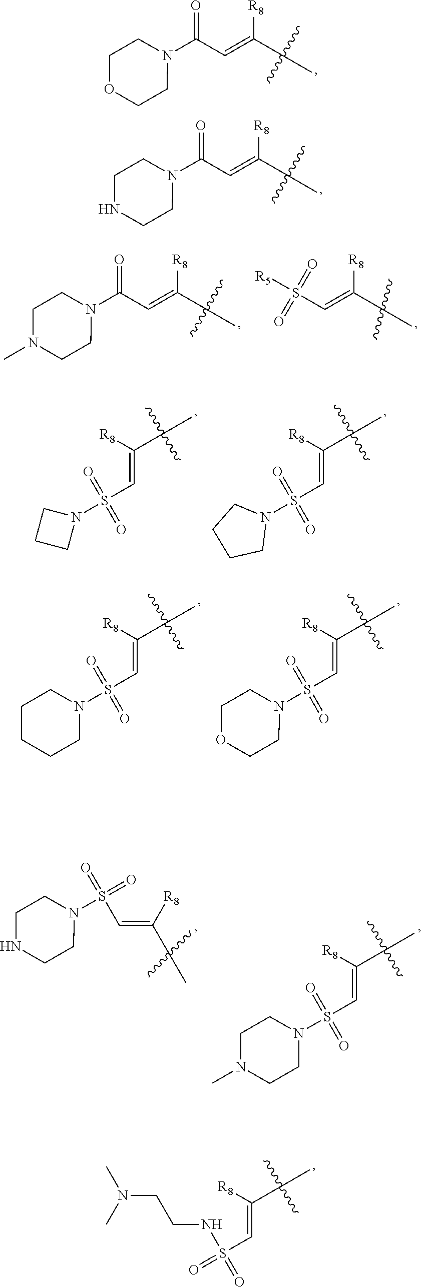
wherein substituents may be at para-, meta-, or ortho- positions on the aromatic ring of Group 1 and Group 2, even if shown in only a single position above; and wherein R3 is a directing moiety to bind and bioconjugate to biological targets, selected from the group consisting of: 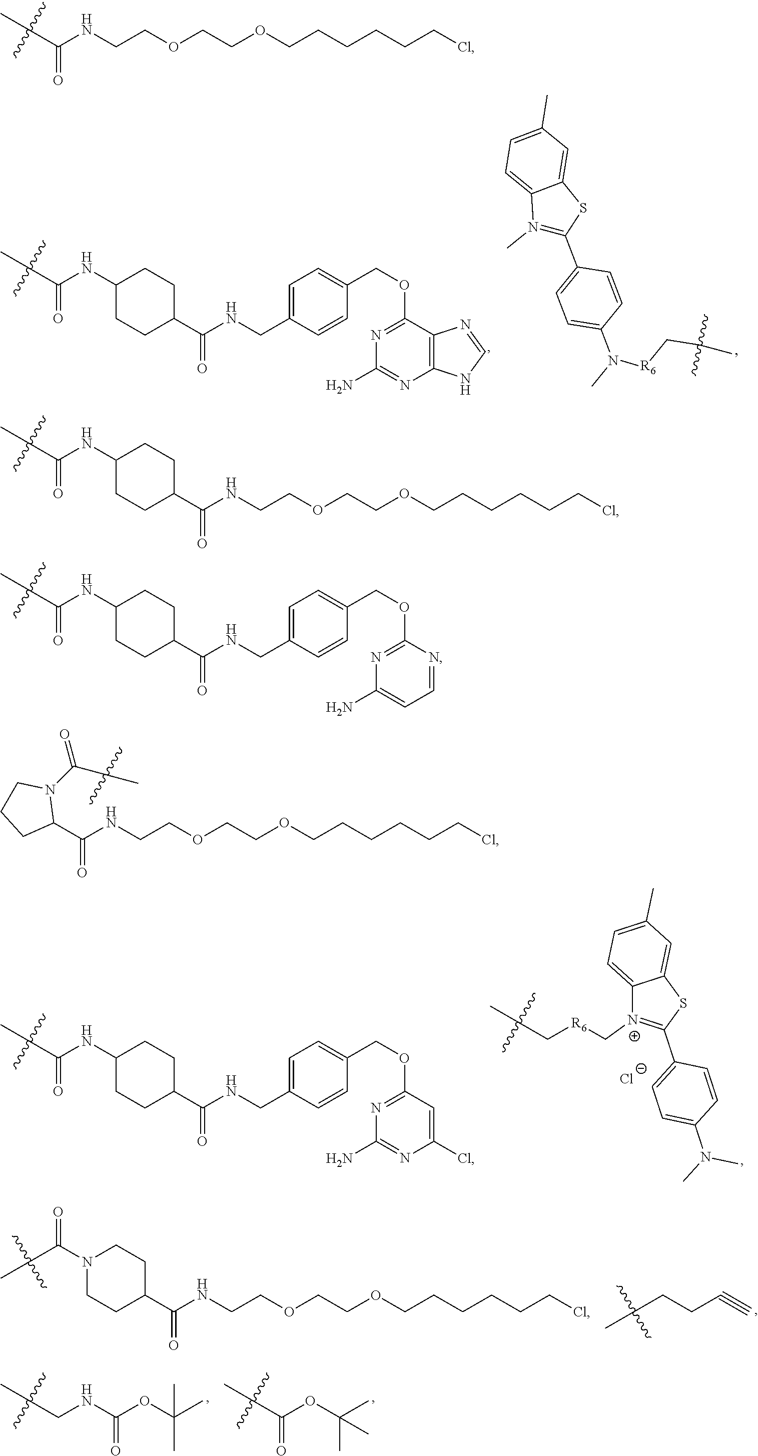 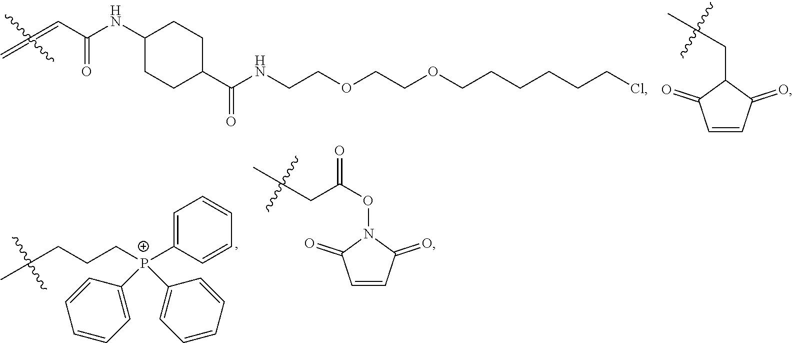
wherein, R₆ is selected from the group consisting of 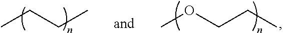
and wherein n is 0, 1, 2, 3, 4 or 5.

16. The method of claim 4, wherein the fluorescent protein chromophore is a compound of Table 1.
